(19) United States
(12) Patent Application Publication
(10) Pub. No.: US 2006/0229302 A1
(43) Pub. Date: Oct. 12, 2006

(54) Tricyclic indole derivatives and their use in the treatment of alzheimer's disease

(76) Inventors: Emmanuel Hubert Demont, Welwyn (GB); Sally Redshaw, Welwyn (GB); Daryl Simon Walter, Welwyn (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/553,878

(22) PCT Filed: Apr. 21, 2004
(86) PCT No.: PCT/EP04/04244

§ 371 Date: Oct. 17, 2005

(30) Foreign Application Priority Data
Apr. 23, 2003 (GB) 03092210

(51) Int. Cl. A61K 31/5415; A61K 31/519; C07D 498/04; C07D 487/04

(52) U.S. Cl. 514/224.5; 514/267; 544/34; 544/252

(57) ABSTRACT

The present invention to novel hydroxyethylamine compounds having Asp2 (β-secretase, BACE1 or Memapsin) inhibitory activit, processes for their preparation, to compositions containing them and to their use in the treatment of diseases characterised by elevated β-amyloid levels or β-amyloid deposits, Particularly Alzheimer's disease.

The present invention relates to novel hydroxyethylamine compounds having Asp2 (β-secretase, BACE1 or Memapsin) inhibitory activity, processes for their preparation, to compositions containing them and to their use in the treatment of diseases characterised by elevated β-amyloid levels or β-amyloid deposits, particularly Alzheimer's disease.

Alzheimer's disease is a degenerative brain disorder in which extracellular deposition of Aβ in the form of senile plaques represents a key pathological hallmark of the disease (Selkoe, D. J. (2001) Physiological Reviews 81: 741-766). The presence of senile plaques is accompanied by a prominent inflammatory response and neuronal loss. β-amyloid (Aβ) exists in soluble and insoluble, fibrillar forms and a specific fibrillar form has been identified as the predominant neurotoxic species (Vassar, R. and Citron, M. (2000) Neuron 27: 419-422). In addition it has been reported that dementia correlates more closely with the levels of soluble amyloid rather than plaque burden (Naslund, J. et al. (2000) J. Am. Med. Assoc. 12: 1571-1577; Younkin, S. (2001) Nat. Med. 1: 8-19). Aβ is known to be produced through the cleavage of the beta amyloid precursor protein (also known as APP) by an aspartyl protease enzyme known as Asp2 (also known as β-secretase, BACE1 or Memapsin) (De Strooper, B. and Konig, G. (1999) Nature 402: 471-472).

Therefore, it has been proposed that inhibition of the Asp2 enzyme would reduce the level of APP processing and consequently reduce the levels of Aβ peptides found within the brain. Therefore, it is also thought that inhibition of the Asp2 enzyme would be an effective therapeutic target in the treatment of Alzheimer's disease.

APP is cleaved by a variety of proteolytic enzymes (De Strooper, B. and Konig, G. (1999) Nature 402: 471-472). The key enzymes in the amyloidogenic pathway are Asp2 (β-secretase) and γ-secretase both of which are aspartic proteinases and cleavage of APP by these enzymes generates Aβ. The non-amyloidogenic, α-secretase pathway, which precludes Aβ formation, has been shown to be catalysed by a number of proteinases, the best candidate being ADAM10, a disintegrin and metalloproteinase. Asp1 has been claimed to show both α- and β-secretase activity in vitro. The pattern of expression of Asp1 and Asp2 are quite different, Asp2 is most highly expressed in the pancreas and brain while Asp1 expression occurs in many other peripheral tissues. The Asp2 knockout mouse indicates that lack of Asp2 abolished Aβ production and also shows that in this animal model endogenous Asp1 cannot substitute for the Asp2 deficiency (Luo, Y. et al. (2001) Nat Neurosci. 4: 231-232; Cai, H. et al. (2001) Nat Neurosci. 4: 233-234; Roberds, S. L. et al. (2001) Hum. Mol. Genet. 10: 1317-1324).

For an agent to be therapeutically useful in the treatment of Alzheimer's disease it is preferable that said agent is a potent inhibitor of the Asp2 enzyme, but should ideally also be selective for Asp2 over other enzymes of the aspartyl proteinase family, e.g Cathepsin D (Connor, G. E. (1998) Cathepsin D in Handbook of Proteolytic Enzymes, Barrett, A. J., Rawlings, N. D., & Woesner, J. F. (Eds) Academic Press London. pp 828-836).

WO 01170672, WO 02/02512, WO 02/02505 and WO 02/02506 (Elan Pharmaceuticals Inc.) describe a series of hydroxyethylamine compounds having β-secretase activity which are implicated to be useful in the treatment of Alzheimer's disease.

We have found a novel series of compounds which are potent inhibitors of the Asp2 enzyme, thereby indicating the potential for these compounds to be effective in the treatment of disease characterised by elevated β-amyloid levels or β-amyloid deposits, such as Alzheimer's disease.

Thus, according to a first aspect of the present invention we provide a compound of formula (I): 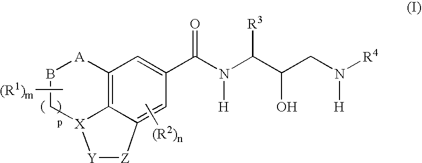
wherein

R¹ and R² independently represent C₁₋₃ alkyl, C₂₋₄ alkenyl, halogen, C₁₋₃ alkoxy, amino, cyano or hydroxy;

m and n independently represent 0, 1 or 2;

p represents 1 or 2;

A-B represents —NR⁵—SO₂— or —NR⁵—CO—;

R⁵ represents hydrogen, C₁₋₆ alkyl, C₃₋₆ alkenyl, C₃₋₆ alkynyl, C₃₋₈ cycloalkyl, aryl, heteroaryl, arylC₁₋₆ alkyl-, heteroarylC₁₋₆ alkyl-, arylC₃₋₈ cycloalkyl- or heteroarylC₃₋₈ cycloalkyl-;

X—Y-Z represents —N—CR⁸═CR⁹—;

R⁸ represents hydrogen, C₁₋₆ alkyl or C₃₋₈ cycloalkyl;

R⁹ represents hydrogen, C₁₋₆ alkyl, C₃₋₈ cycloalkyl, aryl, heteroaryl, arylC₁₋₆ alkyl-, heteroarylC₁₋₆ alkyl-, arylC₃₋₈ cycloalkyl-, heteroarylC₃₋₈ cycloalkyl-, —COOR¹⁰, —OR¹⁰, —CONR¹⁰R¹¹, —SO₂NR¹⁰R¹¹, —COC₁₋₆ alkyl or —SO₂C₁₋₆ alkyl (wherein R¹⁰ and R¹¹ independently represent hydrogen, C₁₋₆ alkyl or C₃₋₈ cycloalkyl);

R³ represents optionally substituted C₁₋₆ alkyl, C₂₋₆ alkenyl, C₂₋₆ alkynyl, —C₁₋₆ alkyl-C₃₋₈ cycloalkyl, —C₁₋₆ alkyl-aryl, —C₁₋₆ alkyl-heteroaryl or —C₁₋₆ alkyl-heterocyclyl;

R⁴ represents hydrogen, optionally substituted C₁₋₁₀ alkyl, C₂₋₆ alkynyl, —C₃₋₈ cycloalkyl, —C₃₋₈ cycloalkenyl, aryl, heteroaryl, heterocyclyl, —C₁₋₆ alkyl-C₃₋₈ cycloalkyl, —C₃₋₈ cycloalkyl-aryl, -heterocyclyl-aryl, —C₁₋₆ alkyl-aryl-heteroaryl, —C(R^{a}R^{b})—CONH—C₁₋₆ alkyl, —C(R^{a}R^{b})—CONH—C₃₋₈ cycloalkyl, —C₁₋₆ alkyl-S—C₁₋₆ alkyl, —C₁₋₆ alkyl-NR^{c}R^{d}, —C(R^{a}R^{b})—C₁₋₆ alkyl, —C(R^{a}R^{b})-aryl, —C(R^{a}R^{b})-heteroaryl, —C(R^{a}R^{b})-heteroaryl-heteroaryl, —C(R^{a}R^{b})—C₁₋₆ alkyl-aryl, —C(R^{a}R^{b})—C₁₋₆ alkyl-heteroaryl, —C(R^{a}R^{b})—C₁₋₆ alkyl-heterocyclyl, —C₁₋₆ alkyl-O—C₁₋₆ alkyl-aryl, —C₁₋₆ alkyl-O—C₁₋₆ alkyl-heteroaryl or —C₁₋₆ alkyl-O—C₁₋₆ alkyl-heterocyclyl;

R^{a} and R^{b} independently represent hydrogen, C₁₋₆ alkyl, C₂₋₆ alkenyl, C₂₋₆ alkynyl or C₃₋₈ cycloalkyl, or R^{a} and R^{b} together with the carbon atom to which they are attached may form a C₃₋₈ cycloalkyl or heterocyclyl group;

R^{c} and R^{d} independently represent hydrogen, C₁₋₆ alkyl, C₂₋₆ alkenyl, C₂₋₆ alkynyl, C₃₋₈ cycloalkyl or R^{c} and R^{d} together with the nitrogen atom to which they are attached may form a nitrogen containing heterocyclyl group;

wherein said aryl, heteroaryl or heterocyclyl groups of R³—R⁵, R⁹ and R^{a}—R^{d} may be optionally substituted by one or more (eg. 1 to 5) C₁₋₆ alkyl, halogen, haloC₁₋₆ alkyl, haloC₁₋₆ alkoxy, oxo, C₁₋₆ alkoxy, C₂₋₆ alkynyl, C₂₋₆ alkenyl, amino, cyano, nitro, —NR²²COR²³, —CONR²²R²³—SO₂R²², —SO₂NR²²R²³, —COOR²², —C₁₋₆ alkyl-NR²²R²³ (wherein R²² and R²³ independently represent hydrogen, C₁₋₆ alkyl or C₃₋₈ m cycloalkyl), —C₁₋₆ alkyl-O—C₁₋₆ alkyl, —C₁₋₆ alkanoyl or hydroxy groups;

and wherein said alkyl and cycloalkyl groups of R¹—R⁵, R⁸—R¹¹, R²²—R²³ and R^{a}—R^{d} may be optionally substituted by one or more (eg. 1 to 6) halogen, C₁₋₆ alkyl, C₁₋₆ alkoxy, C₁₋₆ alkylamino, amino, cyano, hydroxy, carboxy or —COOC₁₋₆ alkyl groups;

or a pharmaceutically acceptable salt or solvate thereof.

In one particular aspect of the present invention, there is provided a compound of formula (I) as defined above wherein:

p represents 2; and

R⁵ represents hydrogen, C₁₋₆ alkyl, C₃₋₈ cycloalkyl, aryl, heteroaryl, arylC₁₋₆ alkyl-, heteroarylC₁₋₆ alkyl, arylC₃₋₈ cycloalkyl or heteroarylC₃₋₈ cycloalkyl; and R³ represents optionally substituted C₁₋₆ alkyl, —C₁₋₆ alkyl-C₃₋₈ cycloalkyl, —C₁₋₆ alkyl-aryl, —C₁₋₆ alkyl-heteroaryl or —C₁₋₆ alkyl-heterocyclyl; and

R⁴ represents hydrogen, optionally substituted C₁₋₁₀ alkyl, —C₃₋₈ cycloalkyl, —C₃₋₈ cycloalkenyl, aryl, heteroaryl, heterocyclyl, —C₁₋₆ alkyl-C₃₋₈ cycloalkyl, —C₃₋₈ cycloalkyl-aryl, -heterocyclyl-aryl, —C₁₋₆ alkyl-aryl-heteroaryl, —C(R^{a}R^{b})—CONH—C₁₋₆ alkyl, —C(R^{a}R^{b})—C₃₋₈ cycloalkyl, —C₁₋₆ alkyl-S—C₁₋₆ alkyl, —C₁₋₆ alkyl-NR^{c}R^{d}, —C(R^{a}R^{b})—C₁₋₆ alkyl, —C(R^{a}R^{b})—aryl, —C(R^{a}R^{b})—C₁₋₆ alkyl-aryl, —C(R^{a}R^{b})—C₁₋₆ alkyl-heteroaryl, —C(R^{a}R^{b})—C₁₋₆ alkyl-heterocyclyl, —C₁₋₆ alkyl-O—C₁₋₆ alkyl-aryl, —C₁₋₆ alkyl-O—C₁₋₆ alkyl-heteroaryl or —C₁₋₆ alkyl-O—C₁₋₆ alkyl-heterocyclyl; and

R^{a} and R^{b} independently represent hydrogen, C₁₋₆ alkyl, or R^{a} and R^{b} together with the carbon atom to which they are attached may form a C₃₋₈ cycloalkyl or heterocyclyl group;

R^{c} and R^{d} independently represent hydrogen, C₁₋₆ alkyl, C₃₋₈ cycloalkyl, or R^{c} and R^{d} together with the nitrogen atom to which they are attached may form a heterocyclyl group;

optional substituents for alkyl and cycloalkyl groups of R³ and R⁴ include one or more (eg. 1, 2 or 3) halogen, C₁₋₆ alkoxy, amino, cyano or hydroxy groups;

and wherein said aryl, heteroaryl or heterocyclyl groups of R³, R⁴, R⁵ and R⁹ may be optionally substituted by one or more (eg. 1, 2 or 3) C₁₋₆ alkyl, halogen, —CF₃, —OCF₃, oxo, C₁₋₆ alkoxy, C₂₋₆ alkynyl, C₂₋₆ alkenyl, amino, cyano, nitro, —NR²²COR²³, —CONR²²R²³—C₁₋₆ alkyl-NR²² R²³ (wherein R²² and R²³ independently represent hydrogen, C₁₋₆ alkyl or C₃₋₈ cycloalkyl), —C₁₋₆ alkyl-O—C₁₋₆ alkyl, —C₁₋₆ alkanoyl or hydroxy groups.

References to alkyl include references to both straight chain and branched chain aliphatic isomers of the corresponding alkyl. It will be appreciated that references to alkenyl and alkynyl shall be interpreted similarly.

References to C₃₋₈ cycloalkyl include references to all alicyclic (including branched) isomers of the corresponding alkyl.

References to ‘aryl’ include references to monocyclic carbocyclic aromatic rings (eg. phenyl) and bicyclic carbocyclic aromatic rings (e.g. naphthyl) or carbocyclic benzofused rings such as a C₃₋₈ cycloalkyl fused to a phenyl ring (eg. dihydroindenyl).

References to ‘heteroaryl’ include references to mono- and bicyclic heterocyclic aromatic rings containing 1-4 hetero atoms selected from nitrogen, oxygen and sulphur. Examples of monocyclic heterocyclic aromatic rings include but are not limited to e.g. thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl, pyridyl, tetrazolyl and the like. Examples of bicyclic heterocyclic aromatic rings include eg. quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like.

References to ‘heterocyclyl’ include references to a 5-7 membered non-aromatic monocyclic ring containing 1 to 3 heteroatoms selected from nitrogen, sulphur or oxygen. Examples of heterocyclic non-aromatic rings include e.g. morpholinyl, piperidinyl, piperazinyl, thiomorpholinyl, oxathianyl, dithianyl, dioxanyl, pyrrolidinyl, dioxolanyl, oxathiolanyl, imidazolidinyl, tetrahydropyranyl, tetrahydrothiopyranyl, pyrazolidinyl and the like.

The term “nitrogen containing heterocyclyl” is intended to represent any heterocyclyl group as defined above which contains a nitrogen atom.

Preferably, A-B represents —NR⁵—SO₂—.

Preferably, R⁵ represents hydrogen, C₁₋₆ alkyl (eg. methyl, ethyl or i-propyl) optionally substituted by one or more (eg. 1, 2 or 3) halogen atoms (eg. trifluoroethyl), carboxy (eg. —CH₂COOH) or —COOC₁₋₆ alkyl groups (eg. —CH₂—COO-t-Bu), aryl (eg. phenyl) or arylC₁₋₆ alkyl- (eg. benzyl). More preferably, R⁵ represents C₁₋₆ alkyl (eg. methyl or ethyl) or aryl (eg. phenyl), especially C₁₋₆ alkyl (eg. methyl or ethyl).

Preferably, m represents 0 or 1, more preferably 0.

When present, R¹ is preferably C₁₋₃ alkyl (eg. methyl).

Preferably, n represents 0.

Preferably, p represents 2.

Preferably, R⁸ represents hydrogen.

Preferably, R⁹ represents hydrogen or C₁₋₆ alkyl (eg. methyl, ethyl, propyl or isopropyl), more preferably C₁₋₆ alkyl (eg. ethyl, propyl or isopropyl).

Preferably, R³ represents —C₁₋₆ alkyl-aryl (eg. benzyl) optionally substituted by one or two halogen atoms (eg. chlorine or fluorine). For example, R³ preferably represents unsubstituted benzyl, 3-chlorobenzyl, 3-fluorobenzyl or 3,5-difluorobenzyl.

Preferably, R⁴ represents

-hydrogen;

—C₁₋₁₀ alkyl (eg. methyl, ethyl, i-propyl, propyl, methylpropyl, dimethylethyl, butyl, 1,5-dimethylhexyl or 1,1,5-trimethylhexyl) optionally substituted by one or more halogen (eg. fluoroethyl, difluoroethyl or pentafluoropropyl) or C₁₋₆ alkoxy (eg. methoxy) groups;

C₂₋₆ alkynyl (eg. propynyl);

—C₃₋₈ cycloalkyl (eg. cyclopropyl, cyclobutyl, cyclopentyl or cyclohexyl) optionally substituted by one or more halogen atoms (eg. fluorine) or C₁₋₆ alkyl groups (eg. methyl);

—C₁₋₆ alkyl-C₃₋₈ cycloalkyl (eg. —CH₂-cyclopropyl);

aryl (eg. dihydroindenyl);

-heterocyclyl (eg. tetrahydropyranyl);

—C(R^{a}R^{b})-aryl (eg. benzyl, 1-methyl-1-phenylethyl or α,α-dimethylbenzyl) optionally substituted (eg. substituted at the 3 and 5 positions) by one or more halogen, cyano, nitro, haloC₁₋₆ alkyl (eg. —CF₃), haloC₁₋₆ alkoxy (eg. —OCF₃), C₁₋₆ alkyl (eg. methyl) or C₁₋₆ alkoxy (eg. methoxy), C₂₋₆ alkynyl, C₂₋₆ alkenyl, amino, —NR²²COR²³, —CONR²²R²³ —SO₂R²², —SO₂NR²²R²³, —COOR²², —C₁₋₆ alkyl-NR²²R²³, —C₁₋₆ alkanoyl or hydroxy groups;

—C(R^{a}R^{b})-heteroaryl (eg. —CH₂-pyrazolyl, —CH₂-pyridinyl, —CH₂-quinoxalinyl, —CH₂-quinolinyl, —CH₂-thienyl, —CH₂-pyrazinyl or —CH₂-isoxazolyl) optionally substituted by one or more C₁₋₆ alkyl (eg. methyl or ethyl), halogen (eg. bromine), haloC₁₋₆ alkyl (eg. trifluoroethyl) or —CONR²²R²³ (eg. —CONHMe) groups;

—C(R^{a}R^{b})-heteroaryl-heteroaryl (eg. —CH₂-pyridinyl-pyridinyl);

—C(R^{a}R^{b})—C₁₋₆ alkyl-aryl (eg. —(CH₂)₂-phenyl);

—C(R^{a}R^{b})—CONH—C₃₋₈ cycloalkyl (eg. C(R^{a}R^{b})—CONH-cyclohexyl); or —C₃₋₈ cycloalkyl-aryl.

More preferably, R⁴ represents

—C₁₋₁₀ alkyl (eg. methyl, ethyl, i-propyl, propyl, methylpropyl, dimethylethyl, butyl, 1,5-dimethylhexyl or 1,1,5-trimethylhexyl) optionally substituted by one or more halogen (eg. fluoroethyl, difluoroethyl or pentafluoropropyl) or C₁₋₆ alkoxy (eg. methoxy) groups;

—C₃₋₈ cycloalkyl (eg. cyclopropyl, cyclobutyl, cyclopentyl or cyclohexyl) optionally substituted by one or more halogen atoms (eg. fluorine) or C₁₋₆ alkyl groups (eg. methyl);

aryl (eg. dihydroindenyl);

-heterocyclyl (eg. tetrahydropyranyl);

—C(R^{a}R^{b})-aryl (eg. benzyl, 1-methyl-1-phenylethyl or α,α-dimethylbenzyl) optionally substituted (eg. substituted at the 3 and 5 positions) by one or more halogen, cyano, haloC₁₋₆ alkyl (eg. —CF₃), haloC₁₋₆ alkoxy (eg. —OCF₃), C₁₋₆ alkyl (eg. methyl) or C₁₋₆ alkoxy (eg. methoxy) groups;

—C(R^{a}R^{b})-heteroaryl (eg. —CH₂-pyrazolyl, —CH₂-pyridinyl, —CH₂-quinoxalinyl, —CH₂-quinolinyl, —CH₂-thienyl, —CH₂-pyrazinyl or —CH₂-isoxazolyl) optionally substituted by one or more C₁₋₆ alkyl (eg. methyl or ethyl), halogen (eg. bromine), haloC₁₋₆ alkyl (eg. trifluoroethyl) or —CONR²²R²³ (eg. —CONHMe) groups; or

—C(R^{a}R^{b})—CONH—C₃₋₆ cycloalkyl (eg. C(R^{a}R^{b})—CONH-cyclohexyl).

Most preferably, R⁴ represents

—C₁₋₁₀ alkyl (eg. 1,1,5-trimethylhexyl);

—C₃₋₈ cycloalkyl (eg. cyclopropyl or cyclohexyl) optionally substituted by one or more halogen atoms (eg. fluorine) or C₁₋₆ alkyl groups (eg. methyl);

aryl (eg. dihydroindenyl);

-heterocyclyl (eg. tetrahydropyranyl);

—C(R^{a}R^{b})-aryl (eg. benzyl or 1,1-dimethyl-phenyl) optionally substituted (eg. substituted at the 3 and 5 positions) by one or more haloC₁₋₆ alkyl (eg. —CF₃), haloC₁₋₆ alkoxy (eg. —OCF₃), C₁₋₆ alkyl (eg. methyl) or C₁₋₆ alkoxy (eg. methoxy) groups;

—C(R^{a}R^{b})-heteroaryl (eg. —CH₂-pyrazolyl, —CH₂-pyridinyl, —CH₂-thienyl or —CH₂-isoxazolyl) optionally substituted by one or more C₁₋₆ alkyl (eg. ethyl), haloC₁₋₆ alkyl (eg. trifluoroethyl) or —CONR²²R²³ (eg. —CONHMe) groups; or

—C(R^{a}R^{b})—CONH—C₃₋₈ cycloalkyl (eg. C(R^{a}R^{b})—CONH-cyclohexyl).

Especially preferably, R⁴ represents

—C₃₋₈ cycloalkyl (eg. cyclopropyl or cyclohexyl) optionally substituted by one or more halogen atoms (eg. fluorine);

-heterocyclyl (eg. tetrahydropyranyl);

—C(R^{a}R^{b})-aryl (eg. benzyl) optionally substituted (eg. substituted at the 3 and 5 positions) by one or more haloC₁₋₆ alkyl (eg. —CF₃), haloC₁₋₆ alkoxy (eg. —OCF₃), C₁₋₆ alkyl (eg. methyl) or C₁₋₆ alkoxy (eg. methoxy) groups;

—C(R^{a}R^{b})-heteroaryl (eg. —CH₂-pyrazolyl, —CH₂-pyridinyl, —CH₂-thienyl or —CH₂-isoxazolyl) optionally substituted by one or more C₁₋₆ alkyl (eg. ethyl), haloC₁₋₆ alkyl (eg. trifluoroethyl) or —CONR²²R²³ (eg. —CONHMe) groups; or

—C(R^{a}R^{b})—CONH—C₃₋₈ cycloalkyl (eg. C(R^{a}R^{b})—CONH-cyclohexyl).

Preferably, R^{a} and R^{b} independently represent hydrogen or methyl, or R^{a} and R^{b} together with the carbon atom to which they are attached form a cyclopropyl or cyclohexyl group. More preferably R^{a} and R^{b} both represent hydrogen, both represent methyl or together with the carbon atom to which they are attached form a cyclopropyl group.

Preferred compounds according to the invention includes examples E1-E106 as shown below, or a pharmaceutically acceptable salt thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic or organic acids e.g. hydrochlorides, hydrobromides, sulphates, phosphates, acetates, benzoates, citrates, nitrates, succinates, lactates, tartrates, fumarates, maleates, 1-hydroxy-2-naphthoates, palmoates, methanesulphonates, p-toluenesulphonates, naphthalenesulphonates, formates or trifluoroacetates. The present invention includes within its scope all possible stoichiometric and non-stoichiometric forms.

The compounds of formula (I) may be prepared in crystalline or non-crystalline form, and, if crystalline, may optionally be solvated, eg. as the hydrate. This invention includes within its scope stoichiometric solvates (eg. hydrates) as well as compounds containing variable amounts of solvent (eg. water).

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof. Preferably, compounds of formula (I) are in the form of a single enantiomer of formula (Ia):

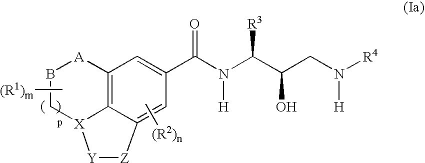

The compounds of formula (I) and salts and solvates thereof may be prepared by the methodology described hereinafter, constituting a further aspect of this invention.

A process according to the invention for preparing a compound of formula (I) which comprises:

(a) reacting a compound of formula (II) 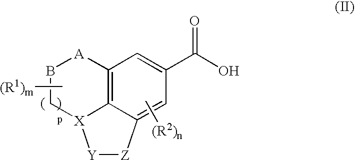
or an activated and/or optionally protected derivative thereof wherein R¹, R², m, n, p, A, B, X, Y and Z are as defined above, with a compound of formula (III) 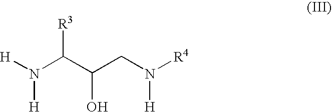
wherein R³ and R⁴are as defined above; or

(b) preparing a compound of formula (I) which comprises reductive alkylation of a compound of formula (IV) 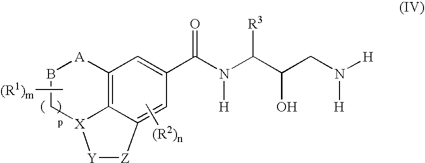
wherein R¹, R², R³, m, n, p, A, B, X, Y and Z are as defined above, with an appropriate aldehyde or ketone; or

(c) deprotecting a compound of formula (I) which is protected; and optionally thereafter

(d) interconversion of compounds of formula (I) to other compounds of formula (I).

Process (a) typically comprises the use of water soluble carbodiimide, HOBT and a suitable base such as tertiary alkylamine or pyridine in a suitable solvent such as DMF and at a suitable temperature, eg. between 0° C. and room temperature.

When process (a) utilises an activated derivative of the compound of formula (II), (eg. by activation of a carboxylic acid to an acid chloride, mixed anhydride, active ester, O-acyl-isourea or other species), process (a) typically comprises treatment of said activated derivative with an amine (Ogliaruso, M. A.; Wolfe, J. F. in The Chemistry of Functional Groups (Ed. Patai, S.) Suppl. B: The Chemistry of Acid Derivatives, Pt. 1 (John Wiley and Sons, 1979), pp 442-8; Beckwith, A. L. J. in The Chemistry of Functional Groups (Ed. Patai, S.) Suppl. B: The Chemistry of Amides (Ed. Zabricky, J.) (John Wiley and Sons, 1970), p 73 ff.

Process (b) typically comprises the use of sodium borohydride triacetate in the presence of a suitable solvent, such as ethanol, dichloromethane and 1,2-dichloroethane and at a suitable temperature, e.g. between 0° C. and room temperature.

In process (c), examples of protecting groups and the means for their removal can be found in T. W. Greene and P. G. M. Wuts ‘Protective Groups in Organic Synthesis’ (J. Wiley and Sons, 3rd Ed. 1999). Suitable amine protecting groups include aryl sulphonyl (e.g. tosyl), acyl (e.g. acetyl), carbamoyl (e.g. benzyloxycarbonyl or t-butoxycarbonyl) and arylalkyl (e.g. benzyl), which may be removed by hydrolysis or hydrogenolysis as appropriate. Other suitable amine protecting groups include trifluoroacetyl (—COCF₃) which may be removed by base catalysed hydrolysis. Suitable hydroxy protecting groups would be silyl based groups such as t-butyldimethylsilyl, which may be removed using standard methods, for example use of an acid such as trifluoroacetic or hydrochloric acid or a fluoride source such as tetra n-butylammonium fluoride.

Process (d) may be performed using conventional interconversion procedures such as epimerisation, oxidation, reduction, alkylation, aromatic substitution, ester hydrolysis, amide bond formation or removal and sulphonylation.

Compounds of formula (II) and/or activated and optionally protected derivatives thereof may be prepared in accordance with the following process: 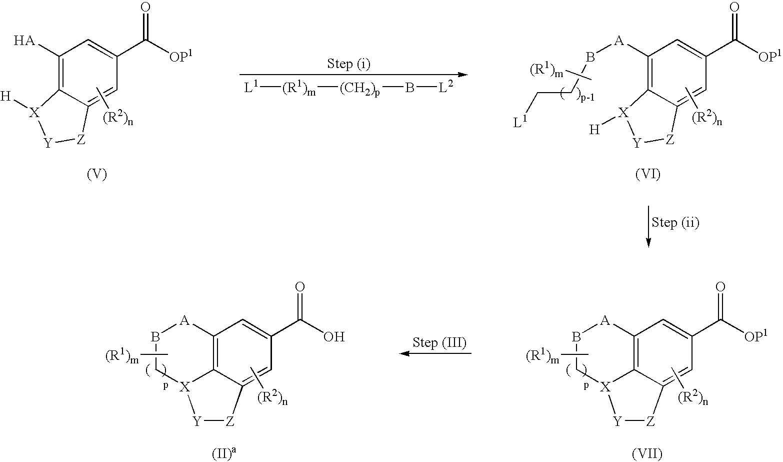
wherein R¹, R², m, n, p, A, B, X, Y and Z are as defined above, P¹ represents a suitable group such as C₁₋₆ alkyl, L¹ and L² independently represent a suitable leaving group such as a halogen atom (eg. chlorine).

When B represents CO, step (i) typically comprises the use of a suitable base such as triethylamine in the presence of a suitable solvent such as dichloromethane at a suitable temperature, such as room temperature.

When B represents SO₂, step (i) typically comprises the use of a suitable base such as pyridine in the presence of a suitable reagent, eg. DMAP and a suitable solvent such as dichloromethane at a suitable temperature, such as room temperature.

When B represents CO, step (ii) typically comprises the use of sodium hydride in the presence of a suitable solvent, eg. dimethylformamide at a suitable temperature, eg. 100° C.

When B represents SO₂, step (ii) typically comprises the use of a suitable base such as triethylamine in the presence of a suitable solvent such as dichloromethane at a suitable temperature, such as room temperature, followed by a subsequent reaction with sodium hydride in the presence of a suitable solvent, eg. dimethylformamide at a suitable temperature, eg. 100° C.

Step (iii) typically comprises a standard procedure for conversion of a carboxylic ester to an acid, such as the use of an appropriate alkali metal hydroxide like lithium or sodium hydroxide in an appropriate solvent such as methanol at an appropriate temperature such as room temperature. In the case of a tert-butyl ester this conversion can be achieved by the use of an appropriate acid such as trifluoroacetic acid in an appropriate solvent such as dichloromethane at an appropriate temperature such as 0° C. Activated derivatives of compounds of formula (II) may then be prepared as described in process (a) above.

Compounds of formula (III) may be prepared in accordance with the following process: 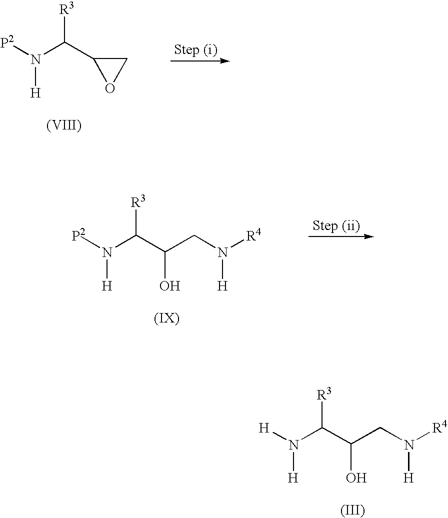
wherein R³ and R⁴ are as defined above and P² represents a suitable amine protecting group, such as t-butoxycarbonyl.

Step (i) typically comprises the reaction of a compound of formula (VIII) with a compound of formula NH₂R⁴ in the presence of a suitable solvent, e.g. ethanol at a suitable temperature, e.g. reflux.

Step (ii) typically comprises the use of suitable deprotection reactions as described above for process (c), eg. when P² represents t-butoxycarbonyl, deprotection typically comprises the use of trifluoroacetic acid in the presence of a suitable solvent, such as dichloromethane at a suitable temperature, e.g. between 0° C. and room temperature.

Compounds of formula (IV) may be prepared in accordance with the following process: 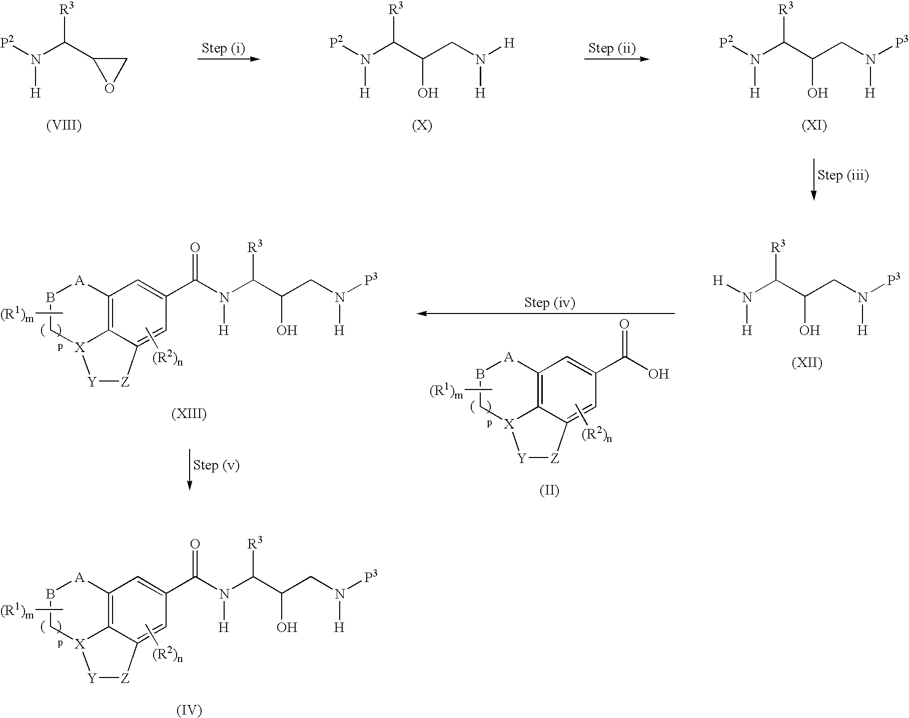
wherein R¹, R², R³, m, n, p, A, B, X, Y, Z and P² are as defined above and P³ represents a suitable amine protecting group different to P², such as —COOCH₂-phenyl.

Step (i) typically comprises the reaction of a compound of formula (VIII) in aqueous ammonia in the presence of a suitable solvent, e.g. ethanol at a suitable temperature, e.g. reflux.

When P³ represents —COOCH₂-phenyl, step (ii) typically comprises the use of ClCOOCH₂-phenyl in the presence of a suitable base, e.g. triethylamine, a suitable solvent, e.g. dimethylformamide at a suitable temperature, e.g. between 0° C. and room temperature.

Step (iii) typically comprises the use of suitable deprotection reactions as described above for process (c), eg. when P² represents t-butoxycarbonyl, deprotection typically comprises the use of trifluoroacetic acid in the presence of a suitable solvent, such as dichloromethane at a suitable temperature, e.g. between 0° C. and room temperature.

Step (iv) typically comprises reacting a compound of formula (XI) with a compound of formula (II) in the presence of water soluble carbodiimide and HOBT.

Step (v) typically comprises the use of suitable deprotection reactions as described above for process (c), eg. when P³ represents —COOCH₂-phenyl, deprotection typically comprises the use of a suitable catalyst, eg. palladium in the presence of a suitable solvent, e.g. water and ethanol and in the presence of a suitable hydrogen source, e.g. ammonium formate at a suitable temperature, eg. 60° C.

Compounds of formula (V) and (VII) are either commercially available or may be prepared from commercially available compounds using standard procedures.

As a further aspect of the invention there is thus provided a compound of formula (I) or a pharmaceutically acceptable salt or solvate thereof for use as a pharmaceutical, particularly in the treatment of patients with diseases characterised by elevated β-amyloid levels or β-amyloid deposits.

According to another aspect of the invention, there is provided the use of a compound of formula (I) or a physiologically acceptable salt or solvate thereof for the manufacture of a medicament for the treatment of patients with diseases characterised by elevated β-amyloid levels or β-amyloid deposits.

In a further or alternative aspect there is provided a method for the treatment of a human or animal subject with diseases characterised by elevated β-amyloid levels or β-amyloid deposits, which method comprises administering to said human or animal subject an effective amount of a compound of formula (I) or a physiologically acceptable salt or solvate thereof.

As a further aspect of the invention there is thus provided a pharmaceutical composition comprising a compound of formula (I) or a pharmaceutically acceptable salt or solvate thereof for use in the treatment of diseases characterised by elevated β-amyloid levels or β-amyloid deposits.

It will be appreciated by those skilled in the art that reference herein to treatment extends to prophylaxis as well as the treatment of diseases characterised by elevated β-amyloid levels or β-amyloid deposits.

The compounds according to the invention may be formulated for administration in any convenient way, and the invention therefore also includes within its scope pharmaceutical compositions for use in the therapy of diseases characterised by elevated β-amyloid levels or β-amyloid deposits, comprising a compound of formula (I) or a physiologically acceptable salt or solvate thereof together, if desirable, with one or more physiologically acceptable diluents or carriers.

It will be appreciated that diseases characterised by elevated β-amyloid levels or β-amyloid deposits include Alzheimer's disease, mild cognitive impairment, Down's syndrome, hereditary cerebral haemorrhage with β-amyloidosis of the Dutch type, cerebral β-amyloid angiopathy and various types of degenerative dementias, such as those associated with Parkinson's disease, progressive supranuclear palsy, cortical basal degeneration and diffuse Lewis body type of Alzheimer's disease.

Most preferably, the disease characterised by elevated β-amyloid levels or β-amyloid deposits is Alzheimer's disease.

There is also provided a process for preparing such a pharmaceutical formulation which comprises mixing the ingredients.

Compounds of formula (I) may be used in combination with other therapeutic agents. Suitable examples of such other therapeutic agents may be acetylcholine esterase inhibitors (such as tetrahydroaminoacridine, donepezil hydrochloride and rivastigmine), gamma secretase inhibitors, anti-inflammatory agents (such as cyclooxygenase II inhibitors), antioxidants (such as Vitamin E and ginkolidesor), statins or p-glycoprotein (P-gp) inhibitors (such as cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979 and PSC-833).

When the compounds are used in combination with other therapeutic agents, the compounds may be administered either sequentially or simultaneously by any convenient route.

The compounds according to the invention may, for example, be formulated for oral, inhaled, intranasal, buccal, enteral, parenteral, topical, sublingual, intrathecal or rectal administration, preferably for oral administration.

Tablets and capsules for oral administration may contain conventional excipients such as binding agents, for example syrup, acacia, gelatin, sorbitol, tragacanth, mucilage of starch, cellulose or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

For buccal administration the compositions may take the form of tablets or lozenges formulated in conventional manner.

The compounds may also be formulated as suppositories, e.g. containing conventional suppository bases such as cocoa butter or other glycerides.

The compounds according to the invention may also be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or tonicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

When the compounds of the invention are administered topically they may be presented as a cream, ointment or patch.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 3000 mg; and such unit doses may be administered more than once a day, for example one, two, three or four times per day (preferably once or twice); and such therapy may extend for a number of weeks, months or years.

All publications, including but not limited to patents and patent applications, cited in this specification are herein incorporated by reference as if each individual publication were specifically and individually indicated to be incorporated by reference herein as though fully set forth.

Preparation of Intermediates

Description 1

### Methyl 4-amino-3-nitrobenzoate (D1)

To a suspension of 4-amino-3-nitrobenzoic acid (50 g, 270 mmol, 1 equiv) in MeOH (600 ml) at room temperature was added SOCl₂ (20 ml, 270 mmol, 1 equiv) dropwise. The resulting suspension was refluxed for 16 h then cooled to room temperature. The suspension was filtered off to give methyl-4-amino-3-nitrobenzoate (D1) (53 g, 100%) as a yellow solid which was used in the next step without further purification. [M+H]⁺=197.3, RT=2.42 min.

Description 2

### Methyl 4-amino-3-bromo-5-nitrobenzoate (D2)

To a solution of methyl-4-amino-3-nitrobenzoate (D1) (48 g, 244 mmol, 1 equiv) in CH₂Cl₂ (1.4 l) at room temperature was added bromine (16.3 ml, 318 mmol, 1.3 equiv). The resulting solution was refluxed for 2 h then another 6 ml (117 mmol, 0.5 equiv) of bromine were added and the solution was stirred for 1 h then cooled to room temperature. The organic phase was washed twice with a 10% sodium thiosulfite aqueous solution (200 ml) then with H₂O (200 ml), dried over MgSO₄ and concentrated in vacuo to give methyl 4-amino-3-bromo-5-nitrobenzoate (D2) (66.2 g, 98%) as a yellow solid which was used in the next step without further purification. [M−H]⁻=274.1, RT=2.90 min.

Description 3

### Methyl 3-bromo-5-nitro-4-[(trifluoroacetyl)amino]benzoate (D3)

To a solution of methyl 4-amino-3-bromo-5-nitrobenzoate (D2) (66 g, 240 mmol, 1 equiv) in CH₂Cl₂ (1.4 l) at 0° C. was added pyridine (100 ml, 720 mmol, 3 equiv) then (CF₃CO)₂O (51 ml, 360 mmol, 1.5 equiv) and the resulting solution was stirred for 1 h. MeOH (29 ml, 720 mmol, 3 equiv) was added and the solution was stirred for 15 min. then concentrated in vacuo. The residue was dissolved in AcOEt (350 ml) and the organic phase was washed three times with a 2N aqueous HCl solution (200 ml). The combined aqueous phases were acidified to pH 1 with concentrated HCl and extracted with AcOEt. The combined organic phases were washed with brine, a saturated NaHCO₃ aqueous solution and brine then dried over MgSO₄ and concentrated in vacuo to give methyl 3-bromo-5-nitro-4-[(trifluoroacetyl)amino]benzoate (D3) (87.2 g, 93%) as a brown oil which was used in the next step without further purification. [M+H]⁺=372.2, RT=2.92 min.

Description 4

### Methyl 3-bromo-4-[(2E/Z)-2-buten-1-yl(trifluoroacetyl)amino]-5-nitrobenzoate (D4)

To a solution of methyl 3-bromo-5nitro-4-[(trifluoroacetyl)amino]benzoate (D3) (84.5 g, 228 mmol, 1 equiv) in CH₃CN (1 l) at room temperature under nitrogen was added K₂CO₃ (37.7 g, 273 mmol, 1.2 equiv) and (2E/Z)-1-bromo-2-butene (30.5 ml, 296 mmol, 1.3 equiv) and the resulting suspension was refluxed for 2 h. (2E/Z)-1-bromo-2-butene (5 ml, 48 mmol, 0.2 equiv) was then added and the suspension refluxed for another hour then cooled to room temperature. The precipitate was filtered off and washed with AcOET and the organic phase concentrated in vacuo. The residue was dissolved in AcOEt and the organic phase was washed with brine, dried over MgSO₄ and concentrated in vacuo to give methyl 3-bromo-4-[(2E/Z)-2-buten-1-yl(trifluoroacetyl)amino]-5-nitrobenzoate (D4) as a brown oil (95 g, 98%) which was used in the next step without further purification. RT=3.70 min.

Descriptions 5 and 6 (D5 and D6)

Descriptions 5 and 6 were obtained using an analogous procedure to that described for Description 4 (D4) from Description 3 (D3) using the appropriate allyl bromide indicated in the table below:

| | Allyl | | RT |
|---|---|---|---|
| Name | bromide | [M + H]⁺ | (min.) |
| Methyl 3-bromo-5-nitro-4-[(2E)- 2-penten-1- yl(trifluoroacetyl)amino]benzoate (D5) | 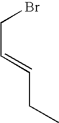 | — | 3.80 |
| Methyl 3-bromo-4-[(3-methyl-2-buten- 1-yl)(trifluoroacetyl)amino]- 5-nitrobenzoate (D6) | 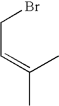 | — | 3.46 |

Description 7

### Methyl 3-ethyl-7-nitro-1H-indole-5-carboxylate and methyl (3Z)-3-ethylidene-7-nitro-2,3-dihydro-1H-indole-5-carboxylate (D7)

To a flask charged with methyl 3-bromo-4-[(2E/Z)-2-buten-1-yl(trifluoroacetyl)amino]-5-nitrobenzoate (D4) (11.1 g, 26.1 mmol, 1 equiv), NaCOOH (1.8 g, 26.1 mmol, 1 equiv), Na₂CO₃ (6.9 g, 65.3 mmol, 2.5 equiv), NBu₄Cl (8 g, 28.7 mmol, 1.1 equiv) and Pd(OAc)₂ (440 mg, 2.0 mmol, 0.075 equiv) at room temperature under nitrogen was added DMF (100 ml) and the resulting mixture was stirred at 100° C. for 1 h then cooled to room temperature. The insoluble material was filtered off and washed with AcOEt and the combined organic phases were concentrated in vacuo. The residue was dissolved in AcOEt and the red precipitate formed (2.6 g) was filtered off. The organic phase was washed with water and brine, dried over MgSO₄ and concentrated in vacuo. The residue was triturated with CH₂Cl₂ and the red precipitate formed (2.1 g) filtered off. The organic phase was concentrated in vacuo and the residue (7 g, black oil) was purified by flash chromatography on silica gel (iso-hexane/AcOEt: 6/4 then 1/1) to give methyl 3-ethyl-7-nitro-1H-indole-5-carboxylate (D7) (1.56 g, 24%) as a pale red solid. All red solids obtained (mixture of D7 and tetrabutyl ammonium salts) were washed with CH₃CN to give a mixture of methyl 3-ethyl-7-nitro-1H-indole-5-carboxylate and methyl (3Z)-3-ethylidene-7-nitro-2,3-dihydro-1H-indole-5-carboxylate (D7) (3.36 g, 52%) which were used in the next step without further purification. [M−H]⁻=247.2, RT=3.42 min.

Descriptions 8-9 (D8-D9)

Descriptions 8-9 were obtained using an analogous procedure to that described for Description 7 from the appropriate precursor indicated in the table below:

| | | | RT |
|---|---|---|---|
| Name | Precursor | [M + H]⁺ | (min.) |
| Methyl 7-nitro-3-propyl-1H-indole-5- | D5 | 263.2 | 3.56 |
| carboxylate (D8) | | | |
| Methyl 3-(1-methylethyl)-7-nitro-1H- | D6 | | |
| indole-5-carboxylate (D9) | | | |

Description 10

### Methyl 7-amino-3-ethyl-1H-indole-5-carboxylate (D10)

To a suspension of methyl 3-ethyl-7-nitro-1H-indole-5-carboxylate and methyl (3Z)-3-ethylidene-7-nitro-2,3-dihydro-1H-indole-5-carboxylate (D7) (3.1 g, 12.5 mmol, 1 equiv) in toluene (150 ml) at room temperature under nitrogen was added palladium on charcoal (10% w/w and 50% wet, 620 mg, 10% w/w) and the resulting suspension was stirred under an atmosphere of hydrogen (1 bar) for 24 h. The catalyst was filtered off through a pad of celite and washed copiously with AcOEt. Th combined organic phases were concentrated in vacuo to give methyl 7-amino-3-ethyl-1H-indole-5-carboxylate (D10) (2.65 g, 97%) as a pale yellow solid which was used in the next step without further purification. [M+H]⁺=219.4, RT=2.82 min.

Descriptions 11-12 (D11-D12)

Descriptions 11-12 (D11-D12) were obtained in an analogous manner to that described for Description 10 from the appropriate precursor indicated in the table below:

| Name | Precursor | [M + H]⁺ | RT (min.) |
|---|---|---|---|
| Methyl 7-amino-3-propyl-1H-indole- | D8 | 233.2 | 3.06 |
| 5-carboxylate (D11) | | | |
| Methyl 7-amino-3-(1-methylethyl)- | D9 | | |
| 1H-indole-5-carboxylate (D12) | | | |

Description 13

### Methyl 7-[(ethenylsulfonyl)amino]-3-ethyl-1H-indole-5-carboxylate (D13)

To a solution of methyl 7-amino-3-ethyl-1H-indole-5-carboxylate (D10) (2.15 g, 9.87 mmol, 1 equiv) in CH₂Cl₂ (70 ml) at room temperature were added pyridine (2 ml, 24.7 mmol, 2.5 equiv), DMAP (120 mg, 0.98 mmol, 0.1 equiv) and 2-chloroethanesulfonyl chloride (1.24 ml, 11.8 mmol, 1.2 equiv) and the resulting mixture was stirred for 12 h then diluted with AcOEt. The organic phase was washed with a 2N aqueous HCl solution, dried over MgSO₄ and concentrated in vacuo to give crude methyl 7-[(ethenylsulfonyl)amino]-3-ethyl-1H-indole-5-carboxylate (D13) (2.98 g, 98%) as a purple solid which was used in the next step without further purification. [M+H]⁺=309.1, RT=3.29 min.

Descriptions 14-15 (D14-D15)

Descriptions 14-15 (D14-D15) were obtained using an analogous manner to that described for Description 13 from the appropriate precursor indicated in the table below:

| Name | Precursor | [M + H]⁺ | RT (min.) |
|---|---|---|---|
| Methyl 7-[(ethenylsulfonyl)amino]- | D11 | 323.4 | 2.98 |
| 3-propyl-1H-indole-5-carboxylate | | | |
| (D14) | | | |
| Methyl 7-[(ethenylsulfonyl)amino]- | D12 | 323.4 | 3.19 |
| 3(1-methylethyl)-1H-indole-5- | | | |
| carboxylate (D15) | | | |

Description 16

### Methyl 7-[(3-chloropropanoyl)amino]-3-ethyl-1H-indole-5-carboxylate (D16)

To a solution of methyl 7-amino-3-ethyl-1H-indole-5-carboxylate (D10) (300 mg, 1.29 mmol, 1 equiv) in CH₂Cl₂ (10 ml) were added NEt₃ (216 μl, 1.55 mmol, 1.2 equiv) and 3-chloropropionyl chloride (136 μl, 1.42 mmol, 1.1 equiv) and the resulting solution was stirred at room temperature for 48 h then diluted with AcOEt and washed with H₂O. The organic phase was dried over MgSO₄ and concentrated in vacuo. Purification of the residue by flash chromatography on silica gel (iso-hexane/AcOEt: 3/1) gave methyl 7-[(3-chloropropanoyl)amino]-3-ethyl-1H-indole-5-carboxylate (D16) (300 mg, 72%) as a white solid. [M+H]⁺=309.4, RT=3.18 min.

Descriptions 17-18 (D17-D18)

Descriptions 17-18 (D17-D18) were obtained using an analogous procedure to that described for Ester 2 (B2) from the appropriate precursor indicated in the table below:

| Name | Precursor | [M + H]⁺ | RT (min.) |
|---|---|---|---|
| Methyl 7-propyl-3,4-dihydro-1H- | D14 | 323.2 | 2.94 |
| [1,2,5]thiadiazepino[3,4,5-hi]indole- | | | |
| 9-carboxylate 2,2-dioxide (D17) | | | |
| Methyl 7-(1-methylethyl)-3,4- | D15 | 323.4 | 2.97 |
| dihydro-1H- | | | |
| [1,2,5]thiadiazepino[3,4,5- | | | |
| hi]indole-9-carboxylate 2,2-dioxide | | | |
| (D18) | | | |

Description 19

### 1,1-Dimethylethyl [(1S,2R)-3-amino-2-hydroxy-1-(phenylmethyl)propyl]carbamate (D19)

To a solution of 1,1-dimethylethyl {(1S)-1-[(2S)-2-oxiranyl]-2-phenylethyl}carbamate (25 g, 95.1 mmol, 1 equiv)) [Chirex 1819W94 Lot#9924382] in MeOH (350 ml) was added aqueous ammonia (32% w/w, 180 ml, 3.2 mol, 3.3 equiv). The resulting mixture was stirred at room temperature for 16 h then concentrated in vacuo to give 1,1-dimethylethyl [(1S,2R)-3-amino-2-hydroxy-1-(phenylmethyl)propyl]carbamate (D19) (25.2 g, 95%) as a white solid which was used in the next step without further purification.

Description 20-25 (D20-D25)

Descriptions 20-25 were obtained using an analogous manner to that described for Example 1 (E1) from the appropriate acid and the appropriate amine indicated in the table below:

| | Acid | Amine | | |
|---|---|---|---|---|
| Description | Precursor | Precursor | [M + H]+ | RT (min) |
| Phenylmethyl ((2R,3S)-4-(3- | A3 | C50 | 653.4 | 3.40 |
| chlorophenyl)-3-{[(7-ethyl-1-methyl-2,2- | | | | |
| dioxido-3,4-dihydro-1H- | | | | |
| [1,2,5]thiadiazepino[3,4,5-hi]indol-9- | | | | |
| yl)carbonyl]amino}-2- | | | | |
| hydroxybutyl)methylcarbamate (D20) | | | | |
| Phenylmethyl [(2R,3S)-3-{[(7-ethyl-1- | A3 | C51 | 637.5 | 3.12 |
| methyl-2,2-dioxido-3,4-dihydro-1H- | | | | |
| [1,2,5]thiadiazepino[3,4,5-hi]indol-9- | | | | |
| yl)carbonyl]amino}-4-(3-fluorophenyl)-2- | | | | |
| hydroxybutyl]methylcarbamate (D21) | | | | |
| Phenylmethyl ((2R,3S)-3-{[(7-ethyl-1- | A3 | C52 | | |
| methyl-2,2-dioxido-3,4-dihydro-1H- | | | | |
| [1,2,5]thiadiazepino[3,4,5-hi]indol-9- | | | | |
| yl)carbonyl]amino}-2-hydroxy-4- | | | | |
| phenylbutyl)methylcarbamate (D22) | | | | |
| Phenylmethyl [(2R,3S)-2-hydroxy-3-({[1- | A9 | C52 | | |
| methyl-7-(1-methylethyl)-2,2-dioxido-3,4- | | | | |
| dihydro-1H-[1,2,5]thiadiazepino[3,4,5- | | | | |
| hi]indol-9-yl]carbonyl}amino)-4- | | | | |
| phenylbutyl]methylcarbamate (D23) | | | | |
| Phenylmethyl ((2R,3S)-3-{[(7-ethyl-1- | A3 | C53 | | |
| methyl-2,2-dioxido-3,4-dihydro-1H- | | | | |
| [1,2,5]thiadiazepino[3,4,5-hi]indol-9- | | | | |
| yl)carbonyl]amino}-2-hydroxy-4- | | | | |
| phenylbutyl)carbamate (D24) | | | | |
| Phenylmethyl ((2R,3S)-3-{[(6-ethyl-1- | A16 | C52 | | |
| methyl-2,2-dioxido-1H- | | | | |
| [1,2,5]thiadiazino[3,4,5-hi]indol-8- | | | | |
| yl)carbonyl]amino}-2-hydroxy-4- | | | | |
| phenylbutyl)methylcarbamate (D25) | | | | |

Description 26

### Methyl 7-{[(chloromethyl)sulfonyl]amino}-3-ethyl-1H-indole-5-carboxylate (D26)

To a solution of methyl 7-amino-3-ethyl-1H-indole-5-carboxylate (D10) (471 mg, 2.16 mmol, 1 equiv) in CH₂Cl₂ (10 ml) at room temperature were added pyridine (260 μl, 3.24 mmol, 1.5 equiv), DMAP (26 mg, 0.22 mmol, 0.1 equiv) and chloromethanesulfonyl chloride (354 mg, 2.4 mmol, 1.1 equiv) and the resulting mixture was stirred for 2 hours then partitioned between AcOEt and a saturated NaHCO₃ aqueous solution. The two layers were separated and the organic phase was washed with H₂O, dried over MgSO₄ and concentrated in vacuo. Trituration of the residue with Et₂O gave methyl 7-{[(chloromethyl)sulfony]amino}-3-ethyl-1H-indole-5-carboxylate (D26) (630 mg, 92%) as a purple solid which was used in the next step without further purification.

Descriptions 27-29 (D27-D29)

Descriptions 27-29 were obtained from (2S)-2-(1-methylethyl)-3,6-bis(methyloxy)-2,5-dihydropyrazine according to the general procedure described in: P. dalla Croce, C. la Rosa, E. Pizzatti Tetrahedron: Asymmetry 2000, 11, 2635-2642:

Name

Methyl 3,5-difluoro-L-phenylalaninate (D27)
Methyl 3-fluoro-L-phenylalaninate (D28)
Methyl 3-chloro-L-phenylalaninate (D29)

Description 30

### Ethyl 2-(3-methoxyphenyl)-2-methylpropanoate (D30)

To a solution of ethyl (3-methoxyphenyl)acetate (19.72 g, 0.101 m, 1 equiv) in THF (200 ml) was added NaH (8.8 g, 0.222 mol, 2.2 equiv) then iodomethane (26 ml, 0.4 mol, 4 equiv). The resulting mixture was stirred at room temperature for 16 h then partitioned between AcOEt and a saturated NaHCO₃ aqueous solution. The two layers were separated and the organic phase washed with brine, dried over MgSO₄ and concentrated in vacuo to give ethyl 2-(3-methoxyphenyl)-2-methylpropanoate (D30) (20.85 g, 98%) as an orange oil which was used in the next step without further purification.

Description 31

### Ethyl 2-methyl-2-[3-(trifluoromethyl)phenyl]propanoate (D31)

Ethyl 2-methyl-2-[3-(trifluoromethyl)phenyl]propanoate (D31) was obtained from ethyl [3-(trifluoromethyl)phenyl]acetate in an analogous manner to the process described for Description 30 (D30).

Description 32

### 2-(3-Methoxyphenyl)-2-methylpropanoic acid (D32)

To a solution of ethyl 2-(3-methoxyphenyl)-2-methylpropanoate (D30) (20.95 g, 94 mmol, 1 equiv) in EtOH (200 ml) was added 2N NaOH aqueous solution (90 ml, 180 mmol, 1.9 equiv) and the resulting mixture was stirred at 70° C. for 16 h then cooled to room temperature. Most of EtOH was removed in vacuo and the residue extracted with AcOEt then acidified to pH 1. The aqueous phase was then extracted with AcOEt and the organic phase dried over MgSO₄ and concentrated in vacuo to give 2-(3-methoxyphenyl)-2-methylpropanoic acid (D32) (15 g, 82%) as a yellow oil which was used in the next step without further purification.

Description 33

### 2-Methyl-2-[3-(trifluoromethyl)phenyl]propanoic acid (D33)

2-Methyl-2-[3-(trifluoromethyl)phenyl]propanoic acid (D33) was obtained from ethyl 2-methyl-2-[3-(trifluoromethyl)phenyl]propanoate (D31) in an analogous manner to the process described for Description 32 (D32).

Description 34

### Benzyl [1-(3-methoxyphenyl)-1-methylethyl]carbamate (D34)

To a solution of 2-(3-methoxyhenyl)-2-methylpropanoic acid (D32) (1 g, 5.15 mmol, 1 equiv) in toluene (20 ml) at room temperature was added NEt₃ (1.07 ml, 7.72 mmol, 1.5 equiv) and then diphenylphosphoryl azide (2.2 ml, 10.3 mmol, 2 equiv). The resulting mixture was then heated at 80° C. for 2 h then benzyl alcohol (1.61 ml, 15.45 mmol, 3 equiv) was added and the solution heated for a further 2 h, cooled to room temperature and partitioned between EtOAc and a saturated NaHCO₃ aqueous solution. The two layers were separated and the aqueous phase dried over MgSO₄ and concentrated in vacuo. Purification of the residue by flash chromatography on silica gel (iso-hexane/AcOEt: 9/1) gave benzyl [1-(3-methoxyphenyl)-1-methylethyl]carbamate (D34) (1 g, 65%) as a yellow gum.

Description 35

### Benzyl {1-methyl-1-[3-(trifluoromethyl)phenyl]ethyl}carbamate (D35)

Benzyl {1-methyl-1-[3-(trifluoromethyl)phenyl]ethyl}carbamate (D35) was obtained from 2-methyl-2-[3-(trifluoromethyl)phenyl]propanoic acid (D33) in an analogous manner to the process described for Description 34 (D34).

Description 36

### 5-Bromo-3-thiophenecarbaldehyde (D36)

To a suspension of 3-thiophenecarbaldehyde (10.6 g, 94.6 mmol, 1 equiv) in CH₂Cl₂ (225 ml) at 0° C. were added AlCl₃ (26.5 g, 199 mmol, 2.1 equiv) and Br₂ (5.1 ml, 99 mmol, 1.05 equiv) and the resulting mixture was refluxed for 7 h then cooled to room temperature. Most of the solvent was removed in vacuo and the residue was poured slowly onto ice. The aqueous phase was extracted twice with AcOEt and the combined organic phases were washed four times with a 2N aqueous HCl solution then with a 10% aqueous NaHSO₃ aqueous solution, a saturated NaHCO₃ aqueous solution, dried over MgSO₄ and concentrated in vacuo. The residue was redissolved in AcOEt and vigorously stirred with a saturated solution of Rochelle's salts for 2 h. The layers were separated and the organic phase dried over MgSO₄ and concentrated in vacuo to give 5-bromo-3-thiophenecarbaldehyde (D36) as a brown oil which was used in the next step without further purification. RT=2.38 min.

Description 37

### 5-Ethenyl-3-thiophenecarbaldehyde (D37)

To a solution of 5-bromo-3-thiophenecarbaldehyde (D36) (2 g, 10.4 mmol, 1 equiv) in DME (45 ml) and H₂O (15 ml) was added tetrakis(triphenylphosphine)-palladium(0) (600 mg, 0.52 mmol, 0.05 equiv), and the suspension was stirred for 10 min. Triethenylboroxin-pyridine complex (prepared according to F. Kerins and D. F. O'Shea in J. Org. Chem, 2002, 67, 4968-4971; 2.64 g, 11 mmol, 1.05 equiv) and K₂CO₃ (1.45 g, 10.5 mmol, 1 equiv) were added and the resulting mixture was stirred at 90° C. for 4 h, cooled to room temperature and diluted with AcOEt. The organic phase was washed with a saturated NaHCO₃ aqueous solution , dried over MgSO₄ and concentrated in vacuo. Purification by flash chromatography on silica gel (iso-hexane/AcOEt: 9/1) gave 5-ethenyl-3-thiophenecarbaldehyde (D37) (660 mg, 100%) of adduct as a pale yellow oil. RT=2.38 min.

Description 38

### 1,1-Dimethylethyl 2-propyn-1-ylcarbamate (D38)

To a solution of 2-propyn-1-amine (2 g, 36.36 mmol, 1 equiv) in CH₂Cl₂ (20 ml) at room temperature were added NEt₃ (5.3 ml, 38.18 mmol, 1.05 equiv) and bis(1,1-dimethylethyl)dicarbonate (8.32 g, 38.18 mmol, 1.05 equiv) and the resulting mixture was stirred at room temperature for 3 h then washed with a 2N aqueous HCl solution and a saturated NaHCO₃ aqueous solution, dried over MgSO₄ and concentrated in vacuo to give 1,1-dimethylethyl 2-propyn-1-ylcarbamate (D38) (4.05 g, 72%) as colourless needles which were used in the next step without further purification.

Description 39

### (1E/Z)-Propanal oxime (D39)

To a solution of hydroxylamine hydrochloride (5 g, 86.2 mmol, 1 equiv) in H₂O (60 ml) were added K₂CO₃ (12.49 g, 90.5 mmol, 1.05 equiv) and propanal (12.49 g, 90.5 mmol, 1.05 equiv) and the resulting mixture was stirred at room temperature for 16 h then extracted 3 times with Et₂O. The combined organic phases were dried over MgSO₄ and concentrated in vacuo to give (1E/Z)-propanal oxime (D39) (4.59 g, 73%) as a clear oil which was used in the next step without further purification.

Description 40

### 1,1-Dimethylethyl [(3-ethyl-5-isoxazolyl)methyl]carbamate (D40)

To a solution of (1E/Z)-propanal oxime (D39) (4 g, 54.8 mmol, 1 equiv) in CH₂Cl₂ (200 ml) was added N-chloro succinimide (7.44 g, 55.8 mmol, 1.02 equiv) and the resulting solution was stirred at room temperature for 2.5 h then pyridine (20 ml, excess) was added and the brown solution stirred for 2 h. 1,1-Dimethylethyl 2-propyn-1-ylcarbamate (D38) (1.36 g, 8.72 mmol, 0.16 equiv) and DIPEA (9.5 ml, 55.8 mmol, 1.02 equiv) were added and the resulting solution was stirred at room temperature for 48 h then washed with a 2N aqueous HCl solution and a saturated NaHCO₃ aqueous solution, dried over MgSO₄ and concentrated in vacuo. Purification of the residue by flash chromatography on silica gel (iso-hexane/AcOEt: 9/1) gave 1,1-dimethylethyl [(3-ethyl-5-isoxazolyl)methyl]carbamate (D40) (1.91 g, 91%) as a clear oil.

Description 41

### N-{3-(Dimethylamino)-2-[(dimethyliminio)methyl]-2-propen-1-ylidene}-N-methylmethanaminium di-tetrafluoro borate salt (D41)

To 100 ml of DMF (1.34 mol, 15 equiv) at 0° C. was added POCl₃ (25.2 ml, 294 mmol, 3.3 equiv) over 2.5 h whilst maintaining the temperature below 4° C. To the resulting pale yellow solution was added bromoacetic acid (12.5 g, 89.9 mmol, 1 equiv) and the mixture is stirred at 90° C. for 5 h then cooled to room temperature and concentrated in vacuo. To the residue was cautiously added 2.5 g of ice at 0° C. followed by sodium tetrafluoroborate (20 g, 182 mmol, 2.0 equiv) in H₂O (40 ml). The solution was cooled to −30° C. and the precipitate formed was filtered off and triturated with CH₃CN to give N-{3-(dimethylamino)-2-[(dimethyliminio)methyl]-2-propen-1-ylidene}-N-methylmethanaminium di-tetrafluoro borate salt (D41) (11.8 g, 33 mmol, 37%) as a white solid which was used in the next step without further purification.

Description 42

### (Hydroxymethylidene)propanedial (D42)

To a solution of N-{3-(dimethylamino)-2-[(dimethyliminio)methyl]-2-propen-1-ylidene}-N-methylmethanaminium di-tetrafluoro borate salt (D41) (11.8 g, 33 mmol, 1 equiv) in H₂O (36 ml) was added K₂CO₃ (1.8 g, 13 mmol, 0.4 equiv) and the resulting mixture was stirred at 40° C. for 5 min. then cooled to room temperature and concentrated HCl (29 ml) was slowly added. The aqueous phase was extracted 5 times with CH₂Cl₂ and the combined organic phases were dried over MgSO₄ and concentrated in vacuo to give (hydroxymethylidene)propanedial (D42) (2.25 g, 68%) as a white solid which was used immediately.

Description 43

### 1-(2,2,2-Trifluoroethyl)-1H-pyrazole-4-carbaldehyde (D43)

To a solution of (hydroxymethylidene)propanedial (D42) (2.25 g, 22.5 mmol, 1 equiv) in MeOH (300 ml) and concentrated HCl (4.4 ml) at room temperature was added (2,2,2-trifluoroethyl)hydrazine hydrochloride (3.39 g, 150 mmol, 6.7 equiv) and the resulting mixture was stirred for 16 h at room temperature then concentrated in vacuo. The residue was partitioned between AcOEt and H₂O and the two layers were separated. The aqueous phase was dried over MgSO₄ and concentrated in vacuo. Purification of the residue by flash chromatography on silica gel (iso-hexane/AcOEt: 4/1 to 1/1) gave 1-(2,2,2-trifluoroethyl)-1H-pyrazole-4-carbaldehyde (D43) (2.8 g, 83%) as a pale yellow oil.

Description 44

### 1,1-Dimethylethyl [(1S)-2-(cyclohexylamino)-1-methyl-2-oxoethyl]carbamate (D44)

N-{[(1,1-dimethylethyl)oxy]carbonyl}-L-alanine (1.5 g, 8.0 mmol, 1 equiv), EDAC.HCl (1.84 g, 9.6 mmol, 1.2 equiv), HOBT (1.47 g, 9.6 mmol, 1.2 equiv), 4-ethylmorpholine (1.76 g, 16 mmol, 2 equiv) and cyclohexylamine (1.1 ml, 9.6 mmol, 1.2 equiv) in CH₂Cl₂ (10 ml) were stirred at room temperature for 16 h. The solution was concentrated in vacuo and the residue dissolved in AcOEt. The organic phase was washed with 2N aqueous HCl solution, saturated aqueous NaHCO₃ solution and brine, dried over MgSO₄ and concentrated in vacuo to give 1,1-dimethylethyl [(1S)-2-(cyclohexylamino)-1-methyl-2-oxoethyl]carbamate (D44) (2.12 g, 98%) as a colourless oil which was used in the next step without further purification.

Description 45

### 4-((Z/E)-But-2-enylamino)-3,5-diiodo-benzoic acid ethyl ester (D45)

To a solution of 4-amino-3,5-diiodo-benzoic acid ethyl ester (commercially available from Maybridge) (72.6 g, 0.17 mmol, 1 equiv) in DMF (450 ml) at 0° C. under nitrogen was added NaH (60% in mineral oil, 7.3 g, 0.18 mmol, 1.05 equiv) portionwise over 2 min. After 10 min crotyl bromide (21.5 ml, 0.21 mmol, 1.2 equiv) in DMF (50 ml) was added via cannula over 5 min and the resulting mixture was allowed to warm to room temperature over 30 min. 5 ml of EtOH were added and the mixture was concentrated in vacuo. The residue was dissolved in AcOEt and the organic phase was washed with H₂O. The aqueous phase was extracted with AcOEt and the combined organic phases were washed with brine, dried over MgSO₄ and concentrated in vacuo to give the title compound (D45) (82 g, 100%) as a pink solid which was used in the next step without further purification. [M+H]⁺=472.0, RT=4.93 min.

Description 46

### 3-Ethyl-7-iodo-1H-indole-5-carboxylic acid ethyl ester (D46)

To a solution of 4-((Z/E)-but-2-enylamino)-3,5-diiodo-benzoic acid ethyl ester (D45) (15 g, 31.8 mmol, 1 equiv) in DMF (150 ml) at room temperature under nitrogen were added Pd(OAc)₂ (357 mg, 1.6 mmol, 0.05 equiv), NaCOOH (6.5 g, 95.6 mmol, 3 equiv), Na₂CO₃ (8.4 g, 79.6 mmol, 2.5 equiv) and Nbu₄Cl (8.0 g, 35.0 mmol, 1.1 equiv). The resulting suspension was stirred under nitrogen at 80° C. for 30 min then cooled to room temperature and concentrated in vacuo. The residue was partitioned between AcOEt and H₂O and the two phases were separated. The organic phase was dried over MgSO₄ and concentrated in vacuo. Purification of the residue by flash chromatography on silica gel (iso-hexane/AcOEt: 9/1) gave the title compound (D46) (6.3 g, 58%) as a white solid. [M+H]⁺=344.0, RT=3.86 min.

Description 47

### 7-Benzyloxycarbonylamino-3-ethyl-1H-indole-5-carboxylic acid ethyl ester (D47)

To a solution of 3-ethyl-7-iodo-1H-indole-5-carboxylic acid ethyl ester (D46) (850 mg, 2.48 mmol, 1 equiv) in toluene (20 ml) at room temperature under nitrogen were added benzyl carbamate (562 mg, 3.72 mmol, 1.5 equiv), copper iodide (24 mg, 0.13 mmol, 0.05 equiv) K₃PO₄ (1.05 g, 4.8 mmol, 2 equiv) and N,N′-dimethylethylenediamine (26 μl, 0.25 mmol, 0.1 equiv) and the resulting suspension was stirred at 100° C. for 30 min then cooled to room temperature and concentrated in vacuo. The residue was partitioned between AcOEt and H₂O and the two phases were separated. The organic phase was dried over MgSO₄ and concentrated in vacuo. Purification of the residue by flash chromatography on silica gel (iso-hexane/AcOEt: 9/1) gave the title compound (D47) (250 mg, 27%) as an off white solid. [M+H]⁺=367.1, RT=3.73 min.

Description 48

### 7-Amino-3-ethyl-1H-indole-5-carboxylic acid ethyl ester (D48)

To a solution of 7-benzyloxycarbonylamino-3-ethyl-1H-indole-5-carboxylic acid ethyl ester (D47) (250 mg, 0.68 mg, 1 equiv) in EtOH (10 ml) were added NH₄COOH (431 mg, 6.8 mmol, 10 equiv), H₂O (2 ml), Pd (10% w/w on charcoal, 50 mg, 0.02 equiv w/w) and the resulting mixture was stirred at 70° C. for 1.5 h. Another 200 mg of Pd (10% w/w on charcoal, 0.08 equiv w/w) were then added and the resulting mixture stirred at 70° C. for another 30 min then cooled to room temperature. The catalyst was filtered off through a pad of celite and most of the EtOH was removed in vacuo. The residue was partitioned between AcOEt and H₂O and the two phases were separated. The organic phase was dried over MgSO₄ and concentrated in vacuo to give the title compound (D48) (150 mg, 95%) as an off white solid which was used in the next step without further purification. [M+H]⁺=233.1, RT=3.19 min.

Description 49

### 7-(3-Chloro-propanoylamino)-3-ethyl-1H-indole-5-carboxylic acid ethyl ester (D49)

To a solution of 7-amino-3-ethyl-1H-indole-5-carboxylic acid ethyl ester (D48) (300 mg, 1.29 mmol, 1 equiv) in CH₂Cl₂ (10 ml) were added NEt₃ (216 μl, 1.55 mmol, 1.2 equiv) and 3-chloropropionyl chloride (136 μl, 1.42 mmol, 1.1 equiv) and the resulting solution was stirred at room temperature for 48 h then diluted with AcOEt and washed with H₂O. The organic phase was dried over MgSO₄ and concentrated in vacuo. Purification of the residue by flash chromatography on silica gel (iso-hexane/AcOEt: 3/1) gave the title compound (D49) (300 mg, 72%) as a white solid. [M+H]⁺=323.4, RT=3.18 min.

Description 50

### 7-Ethenesulfonylamino-3-ethyl-1H-indole-5-carboxylic acid ethyl ester (D50)

To a solution of 7-amino-3-ethyl-1H-indole-5-carboxylic acid ethyl ester (D48) (1.1 g, 4.74 mmol, 1 equiv) in CH₂Cl₂ (20 ml) at room temperature were added pyridine (575 μl, 7.11 mmol, 1.5 equiv), DMAP (66 mg, 0.47 mmol, 0.1 equiv) and 2-chloroethanesulfonyl chloride (545 μl, 5.22 mmol, 1.1 equiv) and the resulting mixture was stirred for 5 min then diluted with AcOEt. The organic phase was washed with a 2N aqueous HCl solution, dried over MgSO₄ and concentrated in vacuo. The residue was dissolved in CH₂Cl₂ (20 ml) and NEt₃ (1 ml, excess) was added and the resulting solution was stirred at room temperature for 16 h then diluted with AcOEt. The organic phase was washed with H₂O, 2N aqueous HCl solution and brine, dried over MgSO₄ and concentrated in vacuo to give crude title compound (D50) (1.7 g, 110%) as a brown oil which was used in the next step without further purification. [M+H]⁺=323.1, RT=3.29 min.

Description 51

### [(1S,2R)-1-Benzyl-2-hydroxy-3-(3-methoxy-benzylamino)-propyl]-carbamic acid tert-butyl ester (D51)

((S)—(S)-1-Oxiranyl-2-phenyl-ethyl)-carbamic acid tert-butyl ester (10 g, 38 mmol, 1 equiv) [Chirex 1819W94 Lot#9924382] was dissolved in EtOH (100 ml) and 3-methoxy-benzylamine (14.6 ml, 114 mmol, 3 equiv) was added. The resulting mixture was heated, under an atmosphere of nitrogen, for 12 h at reflux temperature. The mixture was cooled and the solvent was removed by evaporation in vacuo. The residue was dissolved in AcOEt and washed three times with H₂O, dried over MgSO₄ and concentrated in vacuo. Purification by flash chromatography on silica gel (CH₂Cl₂/MeOH: 98/2 to 95/5) gave the title compound (D51) (10.0 g, 66%) as a white solid.

Description 52

### 1,1-Dimethylethyl (4,4-difluorocyclohexyl)carbamate (D52)

To a solution of 1,1-dimethylethyl (4-oxocyclohexyl)carbamate (3.56 g, 16.7 mmol, 1 equiv) in CH₂Cl₂ (50 ml) was added DAST (4.6 ml, 35.1 mmol, 2.1 equiv) and the resulting mixture was stirred at room temperature for 16 h. A saturated aqueous NaHCO₃ solution (20 ml) was added and the biphasic mixture was vigorously stirred at room temperature for 1 h. The layers were separated and the aqueous phase extracted with CH₂Cl₂. The combined organic layers were dried over MgSO₄ and concentrated in vacuo. Trituration of the residue with hexane gave 1,1-dimethylethyl (4,4-difluorocyclohexyl)carbamate (D52) (1.7 g, 43%) as a white solid which was used in the next step without further purification.

Description F1

### [1-(3-Methoxyphenyl)-1-methylethyl]amine (F1)

A flask was charged with benzyl [1-(3-methoxyphenyl)-1-methylethyl]carbamate (D34) (1 g, 3.34 mmol, 1 equiv), 10% palladium on charcoal (50% wet, 100 mg, 10% w/w), NH₄COOH (2.1 g, 33 mmol, 10 equiv), EtOH (40 ml) and H₂O (8 ml). The resulting mixture was stirred at 80° C. for 2 h, cooled to room temperature and the catalyst was filtered off using a pad of celite. Most of the EtOH was removed in vacuo and the residue was diluted with 1N HCl aqueous solution. The aqueous phase was extracted with AcOEt then basified to pH 13 and extracted twice with AcOEt. These combined organic layers were dried over MgSO₄ and concentrated in vacuo to [1-(3-methoxyphenyl)-1-methylethyl]amine (F1) (290 mg, 53%) as a yellow gum which was used in the next step without further purification.

Description F2

### 2-[3-(Trifluoromethyl)phenyl]propan-2-amine (F2)

2-[3-(Trifluoromethyl)phenyl]propan-2-amine (F2) was obtained from benzyl (1-methyl-1-[3-(trifluoromethyl)phenyl]ethyl}carbamate (D35) in an analogous manner to the process described for Description F1 (F1).

Description F3

### 2,6-Dimethyl-2-heptanamine (F3)

2,6-Dimethyl-2-heptanamine (F3) was obtained according to S. S. Berg and D. T. Cowling, J. Chem. Soc. (C) 1971, 1653-1658.

Description F4

### [(3-Ethyl-5-isoxazolyl)methyl]amine hydrochloride (F4)

1,1-Dimethylethyl [(3-ethyl-5-isoxazolyl)methyl]carbamate (D40) (1.28 g, 5.53 mmol, 1 equiv) was dissolved in a 4M HCl solution in dioxan (20 ml) and the resulting solution was stirred at room temperature for 2 h then concentrated in vacuo. Trituration of the residue with Et₂O gave [(3-ethyl-5-isoxazolyl)methyl]amine hydrochloride (F4) (0.82 g, 92%) as a white solid which was used in the next step without further purification.

Description F5

### N¹-Cyclohexyl-L-alaninamide hydrochloride salt (F5)

N¹-Cyclohexyl-L-alaninamide hydrochloride salt (F5) was obtained from 1,1-dimethylethyl [(1S)-2-(cyclohexylamino)-1-methyl-2-oxoethyl]carbamate (D44) in an analogous manner than for Description F4.

Description F6

### 4,4-Difluorocyclohexanamine tosic salt (F6)

1,1-Dimethylethyl (4,4-difluorocyclohexyl)carbamate (D52) (1.0 g, 4.25 mmol, 1 equiv) was dissolved in CH₃CN (20 ml) and PTSA.H₂O (1.61 g, 8.5 mmol, 2 equiv) was added. The resulting mixture was stirred for 16 h. The precipitate formed was filtered off and triturated with Et₂O to give 4,4-difluorocyclohexanamine tosic salt (F6) (865 mg, 66%) as a white solid which was used in the step without further purification.

Preparation of Epoxides

Epoxide 1

### 1,1-Dimethylethyl {(1S)-2-(3,5-difluorophenyl)-1-[(2S)-2-oxiranyl]ethyl}carbamate (K1)

1,1-Dimethylethyl {(1S)-2-(3,5difluorophenyl)-1-[(2S)-2-oxiranyl]ethyl}carbamate (K1) was obtained from methyl 3,5-difluoro-L-phenylalaninate (D27) according to the procedure described in Patent US 2003/0004360 A1.

Epoxides 2-3 (K2-K3)

Epoxides 2-3 were obtained in an analogous manner to the process described for Epoxide 1 (K1) using the appropriate alaninate indicated in the table below:

| Name | Precursor |
|---|---|
| 1,1-Dimethylethyl {(1S)-2-(3-fluorophenyl)-1-[(2S)-2- | D28 |
| oxiranyl]ethyl}carbamate (K2) | |
| 1,1-Dimethylethyl {(1S)-2-(3-chlorophenyl)-1-[(2S)-2- | D29 |
| oxiranyl]ethyl}carbamate (K3) | |

Preparation of Esters
Ester 1

### Methyl 7-ethyl-2-oxo-1,2,3,4-tetrahydro[1,4]diazepino[3,2,1-hi]indole-9-carboxylate (B1)

To a solution of methyl 7-[(3-chloropropanoyl)amino]-3-ethyl-1H-indole-5-carboxylate (D16) (300 mg, 0.93 mmol, 1 equiv) in DMF (10 ml) at room temperature under nitrogen was added NaH (60% in mineral oil, 41 mg, 1.02 mmol, 1.1 equiv). The resulting solution was heated to 100° C. for 1 h and then cooled to room temperature. Excess NaH was neutralised with MeOH (2 ml) and the solution was concentrated in vacuo. The residue was dissolved in AcOEt and the organic phase was washed with H₂O, dried over MgSO₄ and concentrated in vacuo. Purification of the residue by flash chromatography on silica gel (iso-hexane/AcOEt: 2/3) gave methyl 7-ethyl-2-oxo-1,2,3,4-tetrahydro[1,4]diazepino[3,2,1-hi]indole-9-carboxylate (B1) (120 mg, 45%) as a white solid. [M+H]⁺=273.0, RT=3.08 min.

Ester 2

### Methyl 7-ethyl-3,4-dihydro-1H-[1,2,5]thiadiazepino[3,4,5-hi]indole-9-carboxylate 2,2-dioxide (B2)

To a solution of methyl 7-[(ethenylsulfonyl)amino]-3-ethyl-1H-indole-5-carboxylate (D13) (2.98 g, 9.69 mmol, 1 equiv) in DMF (40 ml) at room temperature under nitrogen was added NaH (60% in mineral oil, 465 mg, 11.6 mmol, 1.2 equiv). After 5 min, the mixture was heated to 100° C. for 1 h and then cooled to room temperature. MeOH (1 ml) was added and the solution was concentrated in vacuo. The residue was dissolved in AcOEt and the organic phase was washed with a saturated NaHCO₃ aqueous solution, dried over MgSO₄ and concentrated in vacuo. Trituration of the residue with Et₂O gave methyl 7-ethyl-3,4-dihydro-1H-[1,2,5]thiadiazepino[3,4,5-hi]indole-9-carboxylate 2,2-dioxide (B2) (1.67 g, 55%) as a brown solid which was used in the next step without further purification. [M+H]⁺=309.3, RT=2.93 min.

Ester 3 (Procedure A)

### Methyl 7-ethyl-1-methyl-3,4-dihydro-1H-[1,2,5]thiadiazepino[3,4,5-hi]indole-9-carboxylate 2,2-dioxide (B3)

To a solution of methyl 7-ethyl-3,4-dihydro-1H-[1,2,5]thiadiazepino[3,4,5-hi]indole-9-carboxylate 2,2-dioxide (B2) (2.07 g, 6.74 mmol, 1 equiv) in DMF (50 ml) at room temperature under nitrogen were added K₂CO₃ (4.65 g, 33.7 mmol, 5 equiv) and iodomethane (2.1 ml, 33.7 mmol, 5 equiv). The resulting mixture was stirred at 80° C. for 1 h then cooled to room temperature, filtered through a pad of celite and concentrated in vacuo. The residue was dissolved in AcOEt and the organic phase was washed with a saturated NaHCO₃ aqueous solution, dried over MgSO₄ and concentrated in vacuo. Trituration of the residue with Et₂O gave methyl 7-ethyl-1-methyl-3,4-dihydro-1H-[1,2,5]thiadiazepino[3,4,5-hi]indole-9-carboxylate 2,2-dioxide (B3) (1.58 g, 73%) as a white solid which was used in the next step without further purification. [M+H]⁺=323.1, RT=2.90 min.

Ester 3 (Procedure B)

### Methyl 7-ethyl-1-methyl-3,4-dihydro-1H-[1,2,5]thiadiazepino[3,4,5-hi]indole-9-carboxylate 2,2-dioxide (B3)

To a solution of methyl 7-ethyl-3,4-dihydro-1H-[1,2,5]thiadiazepino[3,4,5-hi]indole-9-carboxylate 2,2-dioxide (B2) (191 mg, 0.62 mmol, 1 equiv) in DMF (3 ml) at room temperature were added NaH (60% dispersion in mineral oil, 50 mg, 1.25 mmol, 2 equiv) and iodomethane (46 μl, 0.74 mmol, 1.2 equiv) and the resulting solution was stirred for 1 h then partitioned between AcOEt and a saturated NaHCO₃ aqueous solution. The two layers were separated and the organic phase was washed with brine, dried over MgSO₄ and concentrated in vacuo. Purification of the residue by flash chromatography on silica gel (iso-hexane/AcOEt: 4/1 to 1/1) gave methyl 7-ethyl-1-methyl-3,4-dihydro-1H-[1,2,5]thiadiazepino[3,4,5-hi]indole-9-carboxylate 2,2-dioxide (B3) (44 mg, 22%) as a brown gum.

Ester 4

### Methyl 7-ethyl-1-phenyl-3,4-dihydro-1H-[1,2,5]thiadiazepino[3,4,5-hi]indole-9-carboxylate 2,2-dioxide (B4)

To a solution of methyl 7-ethyl-3,4-dihydro-1H-[1,2,5]thiadiazepino[3,4,5-hi]indole-9-carboxylate 2,2-dioxide (B2) (200 mg, 0.65 mmol, 1 equiv) in CH₂Cl₂ (30 ml) were added phenylboronic acid (312 mg, 2.5 mmol, 3.8 equiv), Cu(OAc)₂ (228 mg, 1.25 mmol, 1.9 equiv), NEt₃ (350 μl, 2.5 mmol, 3.8 equiv) and powered activated 4A molecular sieves (300 mg, 150% w/w). The resulting mixture was stirred at room temperature for 2 h then the molecular sieves were filtered off through a pad of celite and the organic phase was washed with 2N HCl aqueous solution and a 2N NaOH aqueous solution, dried over MgSO₄ and concentrated in vacuo. Purification of the residue by flash chromatography on silica gel (iso-hexane/AcOEt: 1/2) gave methyl 7-ethyl-1-phenyl-3,4-dihydro-1H-[1,2,5]thiadiazepino[3,4,5-hi]indole-9-carboxylate 2,2-dioxide (B4) (30 mg, 12%) as a white solid. [M+H]⁺=385.2, R.T.=3.54 min.

Ester 5

### Methyl 7-ethyl-1,3-dimethyl-3,4-dihydro-1H-[1,2,5]thiadiazepino[3,4,5-hi]indole-9-carboxylate 2,2-dioxide (B5)

Methyl 7-ethyl-1,3-dimethyl-3,4-dihydro-1H-[1,2,5]thiadiazepino[3,4,5-hi]indole-9-carboxylate 2,2-dioxide (B5) was obtained as a by-product of the synthesis of Ester 3 (Procedure B).

Esters 9 and 11 (B9 and B11)

Esters 9 and 11 (B9 and B11) were obtained in an analogous manner to that described for Ester 3 (Procedure A) using the appropriate precursor indicated in the table below:

| | | | RT |
|---|---|---|---|
| Name | Precursor | [M + H]⁺ | (min.) |
| Methyl 1-methyl-7-(1-methylethyl)-3,4- | D18 | 337.4 | 3.13 |
| dihydro-1H-[1,2,5]thiadiazepino[3,4,5- | | | |
| hi]indole-9-carboxylate 2,2-dioxide | | | |
| (B9) | | | |
| Methyl 1-methyl-7-propyl-3,4-dihydro- | D17 | 337.2 | 3.13 |
| 1H-[1,2,5]thiadiazepino[3,4,5- | | | |
| hi]indole-9-carboxylate 2,2-dioxide | | | |
| (B11) | | | |

Esters B6-B8, B10 and B12-B13

The following esters were obtained using an analogous manner to that described for Ester 3 (Procedure A) from the appropriate precursor and alkylating reagent indicated in the table below:

| | | Alkylating | | RT |
|---|---|---|---|---|
| Name | Precursor | Reagent | [M + H]⁺ | (min.) |
| Methyl 6-ethyl-1-(phenylmethyl)-9a,9b- dihydro-1H-[1,2,5]thiadiazino[3,4,5- hi]indole-8-carboxylate 2,2-dioxide (B6) | B2 | 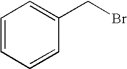 | — | — |
| Methyl 7-ethyl-1-(1-methylethyl)-3,4- dihydro-1H-[1,2,5]thiadiazepino[3,4,5- hi]indole-9-carboxylate 2,2-dioxide (B7) | B2 | 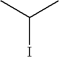 | 351.4 | 3.40 |
| Methyl 1,7-diethyl-3,4-dihydro-1H- [1,2,5]thiadiazepino[3,4,5-hi]indole-9- carboxylate 2,2-dioxide (B8) | B2 | 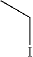 | 337.5 | 3.30 |
| Methyl 7-ethyl-1-(2,2,2-trifluoroethyl)-3,4- dihydro-1H-[1,2,5]thiadiazepino[3,4,5- hi]indole-9-carboxylate 2,2-dioxide (B10) | B2 | 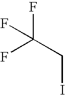 | 391.4 | 3.36 |
| Methyl 1-ethyl-7-propyl-3,4-dihydro-1H- [1,2,5]thiadiazepino[3,4,5-hi]indole-9- carboxylate 2,2-dioxide (B12) | D17 | 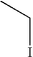 | 351.2 | 3.26 |
| Methyl 1-{2-[(1,1-dimethylethyl)oxy]-2- oxoethyl}-7-ethyl-3,4-dihydro-1H- [1,2,5]thiadiazepino[3,4,5-hi]indole-9- carboxylate 2,2-dioxide (B13) | B2 | 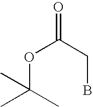 | 440.4 ([M +NH₃ + H]⁺) | 3.46 |

Ester 14

### Methyl 6-ethyl-1H-[1,2,5]thiadiazino[3,4,5-hi]indole-8-carboxylate 2,2-dioxide (B14)

To a solution of methyl 7-{[(chloromethyl)sulfonyl]amino}-3-ethyl-1H-indole-5-carboxylate (D26) (630 mg, 1.91 mmol, 1 equiv) in DMF (10 ml) at room temperature was added NaH (60% dispersion in mineral oil, 153 mg, 3.82 mmol, 2 equiv) and after 5 min the solution was stirred at 100° C. for 1 hour then cooled to room temperature. NaH (60% dispersion in mineral oil, 50 mg, 1.25 mmol, 0.6 equiv) was added and the solution stirred at 100° C. for another 1 h then cooled to room temperature and concentrated in vacuo. The residue was partitioned between AcOEt and a 2N aqueous HCl solution. The two layers were separated and the organic phase was washed with a saturated NaHCO₃ aqueous solution and brine, dried over MgSO₄ and concentrated in vacuo. Purification of the residue by flash chromatography on silica gel (iso-hexane/AcOEt: 9/1 to 1/1) gave methyl 6-ethyl-1H-[1,2,5]thiadiazino[3,4,5-hi]indole-8-carboxylate 2,2-dioxide (B14) (143 mg, 41%) as a brown solid.

Ester 16

### Methyl 6-ethyl-1-methyl-1H-[1,2,5]thiadiazino[3,4,5-hi]indole-8-carboxylate 2,2-dioxide (B16)

To a solution of methyl 6-ethyl-1H-[1,2,5]thiadiazino[3,4,5-hi]indole-8-carboxylate 2,2-dioxide (B14) (27 mg, 91 μmol, 1 equiv) in DMF (1 ml) at room temperature were added NaH (60% dispersion in mineral oil, 7 mg, 0.182 mmol, 2 equiv) and iodomethane (200 μl, 3.2 mmol, excess) and the resulting solution was stirred for 1 h then partitioned between AcOEt and a suturated NaHCO₃ aqueous solution. The two layers were separated and the organic phase was washed with brine, dried over MgSO₄ and concentrated in vacuo. Purification of the residue by flash chromatography on silica gel (iso-hexane/AcOEt: 9/1 to 4/1) gave methyl 6-ethyl-1-methyl-1H-[1,2,5]thiadiazino[3,4,5-hi]indole-8-carboxylate 2,2-dioxide (B16) (20 mg, 71%) as a brown solid. [M+H]⁺=309.1, R.T.=2.90 min.

Ester 15

### Methyl 6-ethyl-1,3-dimethyl-1H-[1,2,5]thiadiazino[3,4,5-hi]indole-8-carboxylate 2,2-dioxide (B15)

Methyl 6-ethyl-1,3-dimethyl-1H-[1,2,5]thiadiazino[3,4,5-hi]indole-8-carboxylate 2,2-dioxide (B15) was obtained as a by-product of the synthesis of ester B16.

Ester 17

### Methyl 6-ethyl-1,3,3-trimethyl-1H-[1,2,5]thiadiazino[3,4,5-hi]indole-8-carboxylate 2,2-dioxide (B17)

Methyl 6-ethyl-1,3,3-trimethyl-1H-[1,2,5]thiadiazino[3,4,5-hi]indole-8-carboxylate 2,2-dioxide (B17) was obtained as a by-product of the synthesis of ester B16.

Ester 18

### 7-Ethyl-2-oxo-1,2,3,4-tetrahydro-[1,4]diazepino[3,2,1-hi]indole-9-carboxylic acid ethyl ester (B18)

To a solution of 7-(3-chloro-propanoylamino)-3-ethyl-1H-indole-5-carboxylic acid ethyl ester (D49) (300 mg, 0.93 mmol, 1 equiv) in DMF (10 ml) at room temperature under nitrogen was added NaH (60% in mineral oil, 41 mg, 1.02 mmol, 1.1 equiv). The resulting solution was heated to 100° C. for 1 h and then cooled to room temperature. Excess NaH was neutralised with EtOH (2 ml) and the solution was concentrated in vacuo. The residue was dissolved in AcOEt and the organic phase was washed with H₂O, dried over MgSO₄ and concentrated in vacuo. Purification of the residue by flash chromatography on silica gel (iso-hexane/AcOEt: 2/3) gave the title compound (B18) (120 mg, 45%) as a white solid. [M+H]⁺=287.0, RT=3.08 min.

Ester 19

### 2-Ethyl-7,7-dioxo-6,7,8,9-tetrahydro-7l⁶-thia-6,9a-diaza-benzo[cd]azulene-4-carboxylic acid ethyl ester (B19)

To a solution of 7-ethenesulfonylamino-3-ethyl-1H-indole-5-carboxylic acid ethyl ester (D50) (130 mg, 0.4 mmol, 1 equiv) in DMF (10 ml) at room temperature under nitrogen was added NaH (60% in mineral oil, 19 mg, 0.45 mmol, 1.2 equiv). After 5 min, the mixture was heated to 100° C. for 1 h and then cooled to room temperature. EtOH (1 ml) was added and the solution was diluted with AcOEt. The organic phase was washed with 2N aqueous HCl solution, dried over MgSO₄ and concentrated in vacuo to give the title compound (B19) (100 mg, 77%) as a brown solid which was used in the next step without further purification. [M+H]⁺=323.3, RT=2.93 min.

Ester 20

### 2-Ethyl-6-methyl-7,7-dioxo-6,7,8,9-tetrahydro-7l⁶-thia-6,9a-diaza-benzo[cd]azulene-4-carboxylic acid ethyl ester (B20)

To a solution of 2-ethyl-7,7-dioxo-6,7,8,9-tetrahydro-7l⁶-thia-6,9a-diaza-benzo[cd]azulene-4-carboxylic acid ethyl ester (B19) (200 mg, 0.621 mmol, 1 equiv) in DMF (10 ml) at room temperature under nitrogen were added NaH (60% in mineral oil, 50 mg, 1.24 mmol, 2 equiv) and, after 2 min, Mel (46 μl, 0.74 mmol, 1.2 equiv). The resulting mixture was stirred at room temperature for 30 min then EtOH (1 ml) was added and the solution concentrated in vacuo. The residue was dissolved in AcOEt and the organic phase was washed with H₂O, dried over MgSO₄ and concentrated in vacuo. Purification of the residue by flash chromatography on silica gel (iso-hexane/AcOEt: 1/1) gave the title compound (B20) (150 mg, 76%) as a white solid. [M+H]⁺=337.1, RT=3.24 min.

Ester 21

### 2-Ethyl-7,7-dioxo-6-phenyl-6,7,8,9-tetrahydro-7l⁶-thia-6,9a-diaza-benzo[cd]azulene-4-carboxylic acid ethyl ester (B21)

To a solution of 2-ethyl-7,7-dioxo-6,7,8,9-tetrahydro-7l⁶-thia-6,9a-diaza-benzo[cd]azulene-4-carboxylic acid ethyl ester (B19) (200 mg, 0.62 mmol, 1 equiv) in CH₂Cl₂ (30 ml) were added phenylboronic acid (312 mg, 2.5 mmol, 4 equiv), copper (II) acetate (220 mg, 1.25 mmol, 2 equiv), NEt₃ (350 ml, 2.5 mmol, 4 equiv) and activated 4 A molecular sieves (300 mg). The resulting mixture was stirred at room temperature for 2 h and then filtered. The filtrate was washed with 2N aqueous HCl solution, a 2N aqueous NaOH solution, dried over MgSO₄ and concentrated in vacuo. Purification of the residue by flash chromatography on silica gel (iso-hexane/AcOEt: 2/1) gave the title compound (B21) (30 mg, 12%) as a white solid. [M+H]⁺=399.2, RT=3.54 min.

Preparation of BOC-Protected Amines

BOC-Protected Amine 1

### Tert-butyl [(1S,2R)-1-benzyl-3-(cyclohexylamino)-2-hydroxypropyl]carbamate (H1)

Tert-butyl {(1S)-1-[(2S)-oxiran-2-yl]-2-phenylethyl}carbamate (10 g, 38 mmol, 1 equiv) [Chirex 1819W94 Lot#9924382] was dissolved in EtOH (100 ml) and cyclohexylamine (13 ml, 114 mmol, 3 equiv) was added. The resulting mixture was heated, under an atmosphere of nitrogen, for 12 h at reflux temperature. The mixture was cooled and the solvent was removed by evaporation in vacuo. The resulting white solid was washed with H₂O and then with Et₂O before drying in vacuo to give tert-butyl [(1S,2R)-1-benzyl-3-(cyclohexylamino)-2-hydroxypropyl]carbamate (H1) (9.0 g, 66%). [M+H]⁺=363.2

BOC-Protected Amines 2-46 (H2-H46)

BOC-protected amines 2-46 were prepared in an analogous manner to that described for BOC-protected amine H1, substituting cyclohexylamine with the appropriate epoxide or amine indicated in the table below (if not commercially available):

| | Epoxide | Amine |
|---|---|---|
| BOC-protected amine | precursor | precursor |
| Tert-butyl {(1S,2R)-1-benzyl-2-hydroxy-3-[(3- | | |
| methoxybenzyl)amino] propyl}carbamate (H2) | | |
| Tert-butyl ((1S,2R)-1-benzyl-2-hydroxy-3-{[3- | | |
| (trifluoromethyl)benzyl] amino}propyl)carbamate (H3) | | |
| Tert-butyl ((1S,2R)-1-benzyl-2-hydroxy-3-{[1-(3- | | F1 |
| methoxyphenyl)-1-methylethyl]amino}propyl)carbamate (H4) | | |
| Tert-butyl [(1S,2R)-1-benzyl-2-hydroxy-3-({1-methyl-1- | | F2 |
| [3-(trifluoromethyl)phenyl] ethyl}amino)propyl] carbamate (H5) | | |
| Tert-butyl ((1S,2R)-1-benzyl-2-hydroxy-3-{[3- | | |
| (trifluoromethoxy)benzyl]amino}propyl)carbamate (H6) | | |
| Tert-butyl [(1S,2R)-1-benzyl-3-(benzylamino)-2- | | |
| hydroxypropyl]carbamate (H7) | | |
| Tert-butyl {(1S,2R)-1-benzyl-2-hydroxy-3-[(2- | | |
| methylbenzyl)amino] propyl}carbamate (H8) | | |
| Tert-butyl {(1S,2R)-1-benzyl-2-hydroxy-3-[(3- | | |
| methylbenzyl)amino] propyl}carbamate (H9) | | |
| Tert-butyl {(1S,2R)-1-benzyl-2-hydroxy-3-[(4- | | |
| methylbenzyl)amino] propyl}carbamate (H10) | | |
| Tert-butyl {(1S,2R)-1-benzyl-2-hydroxy-3-[(pyridin-2- | | |
| ylmethyl)amino]propyl} carbamate (H11) | | |
| Tert-butyl {(1S,2R)-1-benzyl-2-hydroxy-3-[(pyridin-3- | | |
| ylmethyl)amino]propyl} carbamate (H12) | | |
| Tert-butyl {(1S,2R)-1-benzyl-2-hydroxy-3-[(pyridin-4- | | |
| ylmethyl)amino]propyl} carbamate (H13) | | |
| Tert-butyl {(1S,2R)-1-benzyl-2-hydroxy-3-[(2- | | |
| phenylethyl)amino] propyl}carbamate (H14) | | |
| Tert-butyl [(1S,2R)-1-benzyl-2-hydroxy-3-(tetrahydro- | | |
| 2H-pyran-4-ylamino)propyl] carbamate (H15) | | |
| Tert-butyl {(1S,2R)-1-benzyl-3-[(1S)-2,3-dihydro-1H- | | |
| inden-1-ylamino]-2-hydroxypropyl}carbamate (H16) | | |
| Tert-butyl {(1S,2R)-1-benzyl-2-hydroxy-3-[(1,1,5- | | F3 |
| trimethylhexyl)amino] propyl}carbamate (H17) | | |
| Tert-butyl ((1S,2R)-1-benzyl-3-{[(1-ethyl-1H-pyrazol-4- | | |
| yl)methyl]amino}-2-hydroxypropyl)carbamate (H18) | | |
| Tert-butyl {(1S,2R)-1-benzyl-2-hydroxy-3-[(2- | | |
| methoxyethyl)amino] propyl}carbamate (H19) | | |
| Tert-butyl [(1S,2R)-1-benzyl-3-(ethylamino)-2- | | |
| hydroxypropyl]carbamate (H20) | | |
| Tert-butyl {(1S,2R)-1-benzyl-3-[(2-fluoroethyl)amino]-2- | | |
| hydroxypropyl}carbamate (H21) | | |
| Tert-butyl {(1S,2R)-1-benzyl-3-[(2,2- | | |
| difluoroethyl)amino]-2-hydroxypropyl}carbamate (H22) | | |
| Tert-butyl {(1S,2R)-1-benzyl-2-hydroxy-3-[(2,2,2- | | |
| trifluoroethyl)amino] propyl}carbamate (H23) | | |
| Tert-butyl [(1S,2R)-1-benzyl-2-hydroxy-3- | | |
| (propylamino)propyl] carbamate (H24) | | |
| Tert-butyl [(1S,2R)-1-benzyl-2-hydroxy-3- | | |
| (isopropylamino)propyl] carbamate (H25) | | |
| Tert-butyl [(1S,2R)-1-benzyl-3-(cyclopropylamino)-2- | | |
| hydroxypropyl]carbamate (H26) | | |
| Tert-butyl {(1S,2R)-1-benzyl-2-hydroxy-3-[(2,2,3,3,3- | | |
| pentafluoropropyl)amino]propyl}carbamate (H27) | | |
| Tert-butyl [(1S,2R)-1-benzyl-2-hydroxy-3-(prop-2-yn-1- | | |
| ylamino)propyl] carbamate (H28) | | |
| Tert-butyl [(1S,2R)-1-benzyl-3-(butylamino)-2- | | |
| hydroxypropyl]carbamate (H29) | | |
| Tert-butyl ((1S,2R)-1-benzyl-2-hydroxy-3-{[(1S)-1- | | |
| methylpropyl]amino} propyl)carbamate (H30) | | |
| Tert-butyl ((1S,2R)-1-benzyl-2-hydroxy-3-{[(1R)-1- | | |
| methylpropyl]amino} propyl)carbamate (H31) | | |
| Tert-butyl {(1S,2R)-1-benzyl-3-[(cyclopropylmethyl) | | |
| amino]-2-hydroxypropyl}carbamate (H32) | | |
| Tert-butyl [(1S,2R)-1-benzyl-2-hydroxy-3- | | |
| (isobutylamino)propyl] carbamate (H33) | | |
| Tert-butyl [(1S,2R)-1-benzyl-3-(cyclobutylamino)-2- | | |
| hydroxypropyl]carbamate (H34) | | |
| Tert-butyl [(1S,2R)-1-benzyl-3-(Tert-butylamino)-2- | | |
| hydroxypropyl]carbamate (H35) | | |
| Tert-butyl [(1S,2R)-1-benzyl-3-(cyclopentylamino)-2- | | |
| hydroxypropyl]carbamate (H36) | | |
| 1,1-Dimethylethyl [(1S,2R)-3-[(2,2-dimethyltetrahydro- | | |
| 2H-pyran-4-yl)amino]-2-hydroxy-1- | | |
| (phenylmethyl)propyl]carbamate (H37) | | |
| 1,1-Dimethylethyl [(1S,2R)-1-[(3-chlorophenyl)methyl]- | K3 | |
| 3-(cyclopropylamino)-2-hydroxypropyl]carbamate (H38) | | |
| 1,1-Dimethylethyl [(1S,2R)-1-[(3-chlorophenyl)methyl]- | K3 | |
| 3-(cyclohexylamino)-2-hydroxypropyl]carbamate (H39) | | |
| 1,1-Dimethylethyl [(1S,2R)-1-[(3-chlorophenyl)methyl]- | K3 | |
| 2-hydroxy-3-(tetrahydro-2H-pyran-4-ylamino)propyl] | | |
| carbamate (H40) | | |
| 1,1-Dimethylethyl {(1S,2R)-3-(cyclopropylamino)-1-[(3- | K2 | |
| fluorophenyl)methyl]-2-hydroxypropyl}carbamate (H41) | | |
| 1,1-Dimethylethyl {(1S,2R)-3-(cyclohexylamino)-1-[(3- | K2 | |
| fluorophenyl)methyl]-2-hydroxypropyl}carbamate (H42) | | |
| 1,1-Dimethylethyl [(1S,2R)-1-[(3-fluorophenyl)methyl]-2- | K2 | |
| hydroxy-3-(tetrahydro-2H-pyran-4-ylamino)propyl] | | |
| carbamate (H43) | | |
| 1,1-Dimethylethyl {(1S,2R)-3-(cyclopropylamino)-1- | K1 | |
| [(3,5-difluorophenyl)methyl]-2-hydroxypropyl}carbamate | | |
| (H44) | | |
| 1,1-Dimethylethyl {(1S,2R)-3-(cyclohexylamino)-1-[(3,5- | K1 | |
| difluorophenyl)methyl]-2-hydroxypropyl}carbamate | | |
| (H45) | | |
| 1,1-Dimethylethyl [(1S,2R)-1-[(3,5- | K1 | |
| difluorophenyl)methyl]-2-hydroxy-3-(tetrahydro-2H- | | |
| pyran-4-ylamino)propyl] carbamate (H46) | | |

BOC-Protected Amine 47

### 1,1-Dimethylethyl [(1S,2R)-1-[(3-chlorophenyl)methyl]-2-hydroxy-3-(methylamino)propyl]carbamate (H47)

To a solution of methylamine (2N in MeOH, 6 ml, 12 mmol, 7.1 equiv) was added 1,1-dimethylethyl {(1S)-2-(3-chlorophenyl)-1-[(2S)-2-oxiranyl]ethyl}carbamate (K3) (500 mg, 1.68 mmol, 1 equiv) and the resulting mixture was stirred at 60° C. for 10 min with microwaves activation then cooled to room temperature and concentrated in vacuo to give 1,1-dimethylethyl [(1S,2R)-1-[(3-chlorophenyl)methyl]-2-hydroxy-3-(methylamino)propyl]carbamate (H47) (245 mg, 44%) as a cream coloured solid which was used in the next step without further purification. [M+H]⁺=329.4, RT=2.13 min.

BOC-Protected Amines 48-49 (H48-H49)

Boc-protected amines 48-49 were obtained in an analogous manner to the procedure described for BOC-protected Amine 47 using the appropriate epoxide indicated in the table below:

| | Precursor | | |
|---|---|---|---|
| Boc-protected amine | epoxide | [M + H]+ | RT (min) |
| 1,1-Dimethylethyl [(1S,2R)-1-[(3- | K2 | 313.5 | 1.98 |
| fluorophenyl)methyl]-2-hydroxy-3- | | | |
| (methylamino)propyl]carbamate (H48) | | | |
| 1,1-Dimethylethyl [(1S,2R)-2-hydroxy-3- (methylamino)-1- (phenylmethyl)propyl]carbamate (H49) | 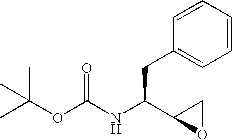 | 295.5 | 1.97 |

BOC-protected Amine 50

### Phenylmethyl [(2R,3S)-4-(3-chlorophenyl)-3-({[(1,1-dimethylethyl)oxy]carbonyl}amino)-2-hydroxybutyl]methylcarbamate (H50)

To a solution of 1,1-dimethylethyl [(1S,2R)-1-[(3-chlorophenyl)methyl]-2-hydroxy-3-(methylamino)propyl]carbamate (H47) (245 mg, 0.75 mmol, 1 equiv)in CH₂Cl₂ (5 ml) at 0° C. were added pyridine (91 ml, 1.12 mmol, 1.5 equiv) and phenylmethyl chloridocarbonate (117 ml, 0.825 mmol, 1.1 equiv) and the resulting solution was stirred at this temperature for 4 h then concentrated in vacuo. Purification of the residue by flash chromatography on silica gel (iso-hexane/AcOet: 4/1 to 1/1) gave phenylmethyl [(2R,3S)-4-(3-chlorophenyl)-3-({[(1,1-dimethylethyl)oxy]carbonyl}amino)-2-hydroxybutyl]methylcarbamate (H50) (227 mg, 66%) as a white foam. [M+H]⁺=463.4, RT=3.58 min.

BOC-Protected Amines 51-52 (H51-H52)

Boc-protected amines H51-H52 were obtained in an analogous manner to the procedure described for BOC-protected Amine 50 using the appropriate precursor indicated in the table below:

| Boc-protected amine | Precursor | [M + H]+ | RT (min) |
|---|---|---|---|
| 1,1-Dimethylethyl [(1S,2R)-1-[(3-fluorophenyl) | H48 | 447.4 | 3.39 |
| methyl]-2-hydroxy-3-(methyl{[(phenylmethyl)oxy] | | | |
| carbonyl}amino)propyl] carbamate (H51) | | | |
| 1,1-Dimethylethyl [(1S,2R)-2-hydroxy-3- | H49 | — | — |
| (methyl{[(phenylmethyl)oxy]carbonyl}amino)-1- | | | |
| (phenylmethyl)propyl]carbamate (H52) | | | |

BOC-Protected Amine 53

### 1,1-Dimethylethyl [(1S,2R)-2-hydroxy-1-(phenylmethyl)-3-({[(phenylmethyl)oxy]carbonyl}amino)propyl]carbamate (H53)

A solution of 1,1-dimethylethyl [(1S,2R)-3-amino-2-hydroxy-1-(phenylmethyl)propyl]carbamate (D19) (25.6 g, 91.4 mmol, 1 equiv) in DMF (250 ml) at 0° C. was treated with NEt₃(15 ml, 108 mmol, 1.2 equiv) and then with benzyl chloroformate (14 ml, 98 mmol, 1.1 equiv) in DMF (50 ml) dropwise. The resulting solution was stirred at 0° C. for 1 h and at room temperature for 16 h and then concentrated in vacuo. The residue was partitioned between AcOEt and saturated aqueous NaHCO₃ solution. The resulting precipitate was diluted with H₂O and filtered to give 1,1-dimethylethyl [(1S,2R)-2-hydroxy-1-(phenylmethyl)-3-({[(phenylmethyl)oxy]carbonyl}amino)propyl]carbamate (H53) (31.5 g, 83%) as a white solid which was used in the next step without further purification.

BOC-Protected Amine 54

### 1,1-Dimethylethyl [(1S,2R)-3-{[(6-bromo-2-pyridinyl)methyl]amino}-2-hydroxy-1-(phenylmethyl)propyl]carbamate (H54)

To a solution of 1,1-dimethylethyl [(1S,2R)-3-amino-2-hydroxy-1-(phenylmethyl)propyl]carbamate (D19) (280 mg, 1 mmol, 1 equiv) in CH₂Cl₂ (6 ml) were added 6-bromo-2-pyridinecarbaldehyde (186 mg, 1 mmol, 1 equiv), AcOH (280 μl, 5 mmol, 5 equiv)) and NaBH(OAc)₃ (848 mg, 4 mmol, 4 equiv) and the resulting mixture was stirred at room temperature for 1 hour then washed with a saturated NaHCO₃ aqueous solution, dried over MgSO₄ and concentrated in vacuo. Purification by flash chromatography on silica gel gave 1,1-dimethylethyl [(1S,2R)-3-[(6-bromo-2-pyridinyl)methyl]amino}-2-hydroxy-1-(phenylmethyl)propyl]carbamate (H54) (360 mg, 80%) as a white solid. [M+H]⁺=450.4, RT=2.44 min.

BOC-Protected Amines 55-61 (H55-H61)

BOC-protected Amines 55-61 (H55-H61) were obtained in an analogous manner to that described for BOC-protected Amine 54 using the appropriate aldehyde indicated in the table below (if not commercially available):

| Boc-protected amine | Aldehyde |
|---|---|
| 1,1-Dimethylethyl [(1S,2R)-3-{[(5-ethenyl-3-thienyl)methyl]amino}-2- | D37 |
| hydroxy-1-(phenylmethyl)propyl]carbamate (H55) | |
| 1,1-Dimethylethyl [(1S,2R)-2-hydroxy-1-(phenylmethyl)-3-({[1-(2,2,2- | D43 |
| trifluoroethyl)-1H-pyrazol-4-yl]methyl}amino)propyl]carbamate (H56) | |
| 1,1-Dimethylethyl [(1S,2R)-2-hydroxy-3-[({5-[(methylamino)carbonyl]- | |
| 3-pyridinyl}methyl)amino]-1-(phenylmethyl)propyl]carbamate (H57) | |
| 1,1-Dimethylethyl [(1S,2R)-3-[(2,2′-bipyridin-6-ylmethyl)amino]-2-hydroxy- | |
| 1-(phenylmethyl)propyl]carbamate (H58) | |
| 1,1-Dimethylethyl [(1S,2R)-2-hydroxy-3-{[(6-methyl-2- | |
| quinoxalinyl)methyl]amino}-1-(phenylmethyl)propyl]carbamate (H59) | |
| 1,1-Dimethylethyl {(1S,2R)-2-hydroxy-1-(phenylmethyl)-3-[(3- | |
| quinolinylmethyl)amino]propyl}carbamate (H60) | |
| 1,1-Dimethylethyl [(1S,2R)-2-hydroxy-3-{[(6-methyl-2- | |
| pyridinyl)methyl]amino}-1-(phenylmethyl)propyl]carbamate (H61) | |

BOC-Protected Amine 62

### 1,1-Dimethylethyl [(1S,2R)-3-{[(5-ethyl-3-thienyl)methyl]amino}-2-hydroxy-1-(phenylmethyl)propyl]carbamate (H62)

To a solution of 1,1-dimethylethyl [(1S,2R)-3-{[(5-ethenyl-3-thienyl)methyl]amino}-2-hydroxy-1-(phenylmethyl)propyl]carbamate (H55) (520 mg, 1.3 mmol, 1 equiv) in EtOH (100 ml) at room temperature were added 10% Palladium on charcoal (50% wet, 260 mg, 25% w/w) and NH₄COOH (1.6 g, 25.4 mmol, 20 equiv) and the resulting mixture was stirred at reflux for 2 h then cooled to room temperature. The catalyst was filtered off through a pad of celite and most of the solvent was removed. The residue was partitioned between AcOEt and a saturated NaHCO₃ aqueous solution and the layers were separated. The organic phase was washed with a saturated NaHCO₃ aqueous solution, dried over MgSO₄ and concentrated in vacuo to give 1,1-dimethylethyl [(1S,2R)-3-{[(5-ethyl-3-thienyl)methyl]amino}-2-hydroxy-1-(phenylmethyl)propyl]carbamate (H62) (410 mg, 79%) as a white solid which was used in the next step without further purification. [M+H]⁺=505.1, RT=2.71 min.

BOC-Protected Amines 63-66 (H63-H66)

BOC-protected amines 63-66 were prepared in an analogous manner to that described for BOC-protected amine H1, substituting cyclohexylamine with the appropriate epoxide or amine indicated in the table below (if not commercially available):

| | Epoxide | Amine |
|---|---|---|
| BOC-protected amine | precursor | precursor |
| 1,1-Dimethylethyl [(1S,2R)-2-hydroxy-3-{[(5-methyl-2- | | |
| pyrazinyl)methyl]amino}-1-(phenylmethyl)propyl]carbamate | | |
| (H63) | | |
| 1,1-Dimethylethyl [(1S,2R)-3-{[(3-ethyl-5-isoxazolyl)methyl] | | F4 |
| amino}-2-hydroxy-1-(phenylmethyl)propyl]carbamate (H64) | | |
| 1,1-Dimethylethyl [(1S,2R)-3-{[(1S)-2-(cyclohexylamino)-1- | | F5 |
| methyl-2-oxoethyl]amino}-2-hydroxy-1-(phenylmethyl)propyl] | | |
| carbamate (H65) | | |
| 1,1-Dimethylethyl [(1S,2R)-3-[(4,4-difluorocyclohexyl) amino]- | | F6 |
| 2-hydroxy-1-(phenylmethyl)propyl]carbamate (H66) | | |

Preparation of Acids
Acid 1

### 7-Ethyl-2-oxo-1,2,3,4-tetrahydro[1,4]diazepino[3,2,1-hi]indole-9-carboxylic acid (A1)

To a solution methyl 7-ethyl-2-oxo-1,2,3,4-tetrahydro[1,4]diazepino[3,2,1-hi]indole-9-carboxylate (B1) (120 mg, 0.42 mmol, 1 equiv) in EtOH (20 ml) was added 2N aqueous NaOH solution (20 ml, 40 mmol, 95 equiv). The resulting mixture was stirred for 14 h then most of EtOH was removed in vacuo. The residue was extracted with Et₂O. The aqueous layer was acidified using 2N aqueous HCl solution and the white precipitate formed was extracted twice with AcOEt. The combined organic solutions were dried over MgSO₄ and concentrated in vacuo to give 7-ethyl-2-oxo-1,2,3,4-tetrahydro-[1,4]diazepino[3,2,1-hi]indole-9-carboxylic acid (A1) (62 mg, 57%) as a white solid, which was used in the next step without further purification. [M+H]⁺=259.4, RT=2.56 min.

Acid 1 (Alternative Procedure)

### 7-Ethyl-2-oxo-1,2,3,4-tetrahydro-[1,4]diazepino[3,2,1-hi]indole-9-carboxylic acid (A1)

To a solution 7-ethyl-2-oxo-1,2,3,4-tetrahydro-[1,4]diazepino[3,2,1-hi]indole-9-carboxylic acid ethyl ester (B18) (120 mg, 0.42 mmol, 1 equiv) in EtOH (20 ml) was added 2N aqueous NaOH solution (20 ml, 40 mmol, 95 equiv). The resulting mixture was stirred for 14 h then most of EtOH was removed in vacuo. The residue was extracted with Et₂O. The aqueous layer was acidified using 2N aqueous HCl solution and the white precipitate formed was extracted twice with AcOEt. The combined organic solutions were dried over MgSO₄ and concentrated in vacuo to give the title compound (A1) (62 mg, 57%) as a white solid, which was used in the next step without further purification. [M+H]⁺=259.4, RT=2.56 min.

Acids 2-17 (A2-A17)

Acids 2-17 were prepared in an analogous manner to that described for Acid 1, from the corresponding esters indicated in the table below:

| Acid | Ester | [M + H]⁺ | RT (min) |
|---|---|---|---|
| 7-Ethyl-3,4-dihydro-1H-[1,2,5]thiadiazepino[3,4,5-hi]indole- | B2 | 293.2 | 2.55 |
| 9-carboxylic acid 2,2-dioxide (A2) | | | |
| 7-Ethyl-1-methyl-3,4-dihydro-1H- | B3 | 309.1 | 2.68 |
| [1,2,5]thiadiazepino[3,4,5-hi]indole-9-carboxylic acid | | | |
| 2,2-dioxide (A3) | | | |
| 7-Ethyl-1-phenyl-3,4-dihydro-1H- | B4 | 371.1 | 3.14 |
| [1,2,5]thiadiazepino[3,4,5-hi]indole-9-carboxylic | | | |
| acid 2,2-dioxide (A4) | | | |
| 7-Ethyl-1,3-dimethyl-3,4-dihydro-1H- | B5 | 323.4 | 2.66 |
| [1,2,5]thiadiazepino[3,4,5-hi]indole-9-carboxylic | | | |
| acid 2,2-dioxide (A5) | | | |
| 7-Ethyl-1-(phenylmethyl)-3,4-dihydro-1H- | B6 | 385.4 | 3.02 |
| [1,2,5]thiadiazepino[3,4,5-hi]indole-9-carboxylic | | | |
| acid 2,2-dioxide (A6) | | | |
| 7-Ethyl-1-(1-methylethyl)-3,4-dihydro-1H- | B7 | 337.4 | 2.80 |
| [1,2,5]thiadiazepino[3,4,5-hi]indole-9-carboxylic | | | |
| acid 2,2-dioxide (A7) | | | |
| 1,7-Diethyl-3,4-dihydro-1H- | B8 | 323.4 | 2.70 |
| [1,2,5]thiadiazepino[3,4,5-hi]indole-9-carboxylic | | | |
| acid 2,2-dioxide (A8) | | | |
| 1-Methyl-7-(1-methylethyl)-3,4-dihydro-1H- | B9 | 323.4 | 2.75 |
| [1,2,5]thiadiazepino[3,4,5-hi]indole-9-carboxylic | | | |
| acid 2,2-dioxide (A9) | | | |
| 7-Ethyl-1-(2,2,2-trifluoroethyl)-3,4-dihydro-1H- | B10 | 377.3 | 2.82 |
| [1,2,5]thiadiazepino[3,4,5-hi]indole-9-carboxylic | | | |
| acid 2,2-dioxide (A10) | | | |
| 1-Methyl-7-propyl-3,4-dihydro-1H- | B11 | 323.2 | 2.74 |
| [1,2,5]thiadiazepino[3,4,5-hi]indole-9-carboxylic | | | |
| acid 2,2-dioxide (A11) | | | |
| 1-Ethyl-7-propyl-3,4-dihydro-1H- | B12 | 337.2 | 2.86 |
| [1,2,5]thiadiazepino[3,4,5-hi]indole-9-carboxylic | | | |
| acid 2,2-dioxide (A12) | | | |
| 1-{2-[(1,1-Dimethylethyl)oxy]-2-oxoethyl}-7-ethyl- | B13 | 353.3 (- | 2.48 |
| 3,4-dihydro-1H-[1,2,5]thiadiazepino[3,4,5-hi]indole- | | tBu) | |
| 9-carboxylic acid 2,2-dioxide (A13) | | | |
| 6-Ethyl-1H-[1,2,5]thiadiazino[3,4,5-hi]indole-8- | B14 | 279.3 | 2.45 |
| carboxylic acid 2,2-dioxide (A14) | | | |
| 6-Ethyl-1,3-dimethyl-1H-[1,2,5]thiadiazino[3,4,5- | B15 | 309.4 | 2.80 |
| hi]indole-8-carboxylic acid 2,2-dioxide (A15) | | | |
| 6-Ethyl-1-methyl-1H-[1,2,5]thiadiazino[3,4,5- | B16 | 295.4 | 2.70 |
| hi]indole-8-carboxylic acid 2,2-dioxide (A16) | | | |
| 6-Ethyl-1,3,3-trimethyl-1H-[1,2,5]thiadiazino[3,4,5- | B17 | | |
| hi]indole-8-carboxylic acid 2,2-dioxide (A17) | | | |

Acids 2-4 (A2-A4)

Acids 2-4 were obtained from the corresponding esters using an analogous procedure to that described for Acid 1 (Alternative Procedure):

| | Starting | | RT |
|---|---|---|---|
| Acid | Material | [M + H]⁺ | (min) |
| 2-Ethyl-7,7-dioxo-6,7,8,9-tetrahydro-7/⁶- | B19 | 293.2 | 2.55 |
| thia-6,9a-diaza-benzo[cd]azulene-4- | | | |
| carboxylic acid (A2) | | | |
| 2-Ethyl-6-methyl-7,7-dioxo-6,7,8,9- | B20 | 309.1 | 2.68 |
| tetrahydro-7/⁶-thia-6,9a-diaza- | | | |
| benzo[cd]azulene-4-carboxylic | | | |
| acid (A3) | | | |
| 2-Ethyl-7,7-dioxo-6-phenyl-6,7,8,9- | B21 | 371.1 | 3.14 |
| tetrahydro-7/⁶-thia-6,9a-diaza- | | | |
| benzo[cd]azulene-4-carboxylic | | | |
| acid (A4) | | | |

Preparation of Amines
Amine 1

### (2R,3S)-3-Amino-1-(cyclohexylamino)-4-phenylbutan-2-ol di-hydrochloride (C1)

Tert-butyl [(1S,2R)-1-benzyl-3-(cyclohexylamino)-2-hydroxypropyl]carbamate (H1) (9 g, 25 mmol, 1 equiv) was dissolved in MeOH (70 ml) and then a 4M solution of HCl in dioxane (60 ml, excess) was added. The resulting mixture was stirred for 3 h at room temperature and then the solvents were removed by evaporation in vacuo. The resulting residue was washed with AcOEt and then with Et₂O before drying in vacuo to give (2R,3S)-3-amino-1-(cyclohexylamino)-4-phenylbutan-2-ol di-hydrochloride (C1) as a white solid (7.4 g, 88%).

Amines 2-46 (C2-C46)

Amines 2-46 were prepared in an analogous manner to that described for Amine 1 (C1), from BOC-protected amines H2-H46, respectively. In some cases the 4M HCl in dioxane was replaced with 3 equivalents of p-toluene sulphonic acid to give the tosic acid salts as the product.

| Amine | Precursor |
|---|---|
| (2R,3S)-3-Amino-1-[(3-methoxybenzyl)amino]-4-phenylbutan-2-ol di- | H2 |
| tosylate (C2) | |
| (2R,3S)-3-Amino-4-phenyl-1-{[3-(trifluoromethyl)benzyl]amino}butan-2- | H3 |
| ol di-hydrochloride (C3) | |
| (2R,3S)-3-Amino-1-{[1-(3-methoxyphenyl)-1-methylethyl]amino}-4- | H4 |
| phenylbutan-2-ol di-hydrochloride (C4) | |
| (2R,3S)-3-Amino-1-({1-methyl-1-[3- | H5 |
| (trifluoromethyl)phenyl]ethyl}amino)-4-phenylbutan-2-ol di- | |
| hydrochloride (C5) | |
| (2R,3S)-3-Amino-4-phenyl-1-{[3-(trifluoromethoxy)benzyl]amino}butan- | H6 |
| 2-ol di-tosylate (C6) | |
| (2R,3S)-3-Amino-1-(benzylamino)-4-phenylbutan-2-ol di-tosylate (C7) | H7 |
| (2R,3S)-3-Amino-1-[(2-methylbenzyl)amino]-4-phenylbutan-2-ol di- | H8 |
| tosylate (C8) | |
| (2R,3S)-3-Amino-1-[(3-methylbenzyl)amino]-4-phenylbutan-2-ol di- | H9 |
| tosylate (C9) | |
| (2R,3S)-3-Amino-1-[(4-methylbenzyl)amino]-4-phenylbutan-2-ol di- | H10 |
| tosylate (C10) | |
| (2R,3S)-3-Amino-4-phenyl-1-[(pyridin-2-ylmethyl)amino]butan-2-ol tri- | H11 |
| tosylate (C11) | |
| (2R,3S)-3-Amino-4-phenyl-1-[(pyridin-3-ylmethyl)amino]butan-2-ol di- | H12 |
| tosylate (C12) | |
| (2R,3S)-3-Amino-4-phenyl-1-[(pyridin-4-ylmethyl)amino]butan-2-ol di- | H13 |
| tosylate (C13) | |
| (2R,3S)-3-Amino-4-phenyl-1-[(2-phenylethyl)amino]butan-2-ol di- | H14 |
| tosylate (C14) | |
| (2R,3S)-3-Amino-4-phenyl-1-(tetrahydro-2H-pyran-4-ylamino)butan-2- | H15 |
| ol di-hydrochloride (C15) | |
| (2R,3S)-3-Amino-1-[(1S)-2,3-dihydro-1H-inden-1-ylamino]-4- | H16 |
| phenylbutan-2-ol di-tosylate (C16) | |
| (2R,3S)-3-Amino-4-phenyl-1-[(1,1,5-trimethylhexyl)amino]butan-2-ol di- | H17 |
| hydrochloride (C17) | |
| (2R,3S)-3-Amino-1-{[(1-ethyl-1H-pyrazol-4-yl)methyl]amino}-4- | H18 |
| phenylbutan-2-ol di-tosylate (C18) | |
| (2R,3S)-3-Amino-1-[(2-methoxyethyl)amino]-4-phenylbutan-2-ol di- | H19 |
| tosylate (C19) | |
| (2R,3S)-3-Amino-1-ethylamino-4-phenylbutan-2-ol di-tosylate (C20) | H20 |
| (2R,3S)-3-Amino-1-[(2-fluoroethyl)amino]-4-phenylbutan-2-ol di- | H21 |
| tosylate (C21) | |
| (2R,3S)-3-Amino-1-[(2,2-difluoroethyl)amino]-4-phenylbutan-2-ol di- | H22 |
| tosylate (C22) | |
| (2R,3S)-3-Amino-4-phenyl-1-[(2,2,2-trifluoroethyl)amino]butan-2-ol di- | H23 |
| tosylate (C23) | |
| (2R,3S)-3-Amino-4-phenyl-1-(propylamino)butan-2-ol di-tosylate (C24) | H24 |
| (2R,3S)-3-Amino-1-(isopropylamino)-4-phenylbutan-2-ol di-tosylate | H25 |
| (C25) | |
| (2R,3S)-3-Amino-1-(cyclopropylamino)-4-phenylbutan-2-ol di-tosylate | H26 |
| (C26) | |
| (2R,3S)-3-Amino-1-[(2,2,3,3,3-pentafluoropropyl)amino]-4- | H27 |
| phenylbutan-2-ol di-tosylate (C27) | |
| (2R,3S)-3-Amino-4-phenyl-1-(prop-2-yn-1-ylamino)butan-2-ol di- | H28 |
| tosylate (C28) | |
| (2R,3S)-3-Amino-1-(butylamino)-4-phenylbutan-2-ol di-tosylate (C29) | H29 |
| (2R,3S)-3-Amino-1-{[(1S)-1-methylpropyl]amino}-4-phenylbutan-2-ol | H30 |
| di-tosylate (C30) | |
| (2R,3S)-3-Amino-1-{[(1R)-1-methylpropyl]amino}-4-phenylbutan-2-ol | H31 |
| di-tosylate (C31) | |
| (2R,3S)-3-Amino-1-[(cyclopropylmethyl)amino]-4-phenylbutan-2-ol di- | H32 |
| tosylate (C32) | |
| (2R,3S)-3-Amino-1-(isobutylamino)-4-phenylbutan-2-ol di-tosylate | H33 |
| (C33) | |
| (2R,3S)-3-Amino-1-(cyclobutylamino)-4-phenylbutan-2-ol di-tosylate | H34 |
| (C34) | |
| (2R,3S)-3-Amino-1-(tert-butylamino)-4-phenylbutan-2-ol di-tosylate | H35 |
| (C35) | |
| (2R,3S)-3-Amino-1-(cyclopentylamino)-4-phenylbutan-2-ol di-tosylate | H36 |
| (C36) | |
| (2R,3S)-3-Amino-1-[(2,2-dimethyltetrahydro-2H-pyran-4-yl)amino]-4- | H37 |
| phenyl-2-butanol di-tosylate (C37) | |
| (2R,3S)-3-Amino-4-(3-chlorophenyl)-1-(cyclopropylamino)-2-butanol | H38 |
| di-tosylate (C38) | |
| (2R,3S)-3-Amino-4-(3-chlorophenyl)-1-(cyclohexylamino)-2-butanol di- | H39 |
| tosylate (C39) | |
| (2R,3S)-3-Amino-4-(3-chlorophenyl)-1-(tetrahydro-2H-pyran-4- | H40 |
| ylamino)-2-butanol di-tosylate (C40) | |
| (2R,3S)-3-Amino-1-(cyclopropylamino)-4-(3-fluorophenyl)-2-butanol di- | H41 |
| tosylate (C41) | |
| (2R,3S)-3-Amino-1-(cyclohexylamino)-4-(3-fluorophenyl)-2-butanol di- | H42 |
| tosylate (C42) | |
| (2R,3S)-3-Amino-4-(3-fluorophenyl)-1-(tetrahydro-2H-pyran-4- | H43 |
| ylamino)-2-butanol di-tosylate (C43) | |
| (2R,3S)-3-Amino-1-(cyclopropylamino)-4-(3,5-difluorophenyl)-2- | H44 |
| butanol di-tosylate (C44) | |
| (2R,3S)-3-Amino-1-(cyclohexylamino)-4-(3,5-difluorophenyl)-2-butanol | H45 |
| di-tosylate (C45) | |
| (2R,3S)-3-Amino-4-(3,5-difluorophenyl)-1-(tetrahydro-2H-pyran-4- | H46 |
| ylamino)-2-butanol di-tosylate (C46) | |

Amines 50-52 (C50-C52)

Amines 50-52 were obtained in an analogous procedure to that described for Amine 53 (C53) from BOC-protected amines H50-H52, respectively:

| | | | RT |
|---|---|---|---|
| Amine | Precursor | [M + H]⁺ | (min) |
| Phenylmethyl [(2R,3S)-3-amino-4-(3- | H50 | 363.4 | 2.27 |
| chlorophenyl)-2- | | | |
| hydroxybutyl]methylcarbamate | | | |
| hydrochloride (C50) | | | |
| Phenylmethyl [(2R,3S)-3-amino-4-(3- | H51 | 347.5 | 2.05 |
| fluorophenyl)-2- | | | |
| hydroxybutyl]methylcarbamate | | | |
| hydrochloride (C51) | | | |
| Phenylmethyl [(2R,3S)-3-amino-2- | H52 | — | — |
| hydroxy-4-phenylbutyl]methyl carbamate | | | |
| hydrochloride (C52) | | | |

### Phenylmethyl [(2R,3S)-3-amino-2-hydroxy-4-phenylbutyl]carbamate hydrochloride (C53)

A solution of 1,1-dimethylethyl [(1S,2R)-2-hydroxy-1-(phenylmethyl)-3-({[(phenylmethyl)oxy]carbonyl}amino)propyl]carbamate (H53) (31.5 g, 76.1 mmol, 1 equiv) in THF (300 ml) was treated with 4N HCl solution in dioxan (40 ml, 160 mmol, 2.1 equiv). The resulting solution was stirred at room temperature for 2 h then concentrated in vacuo. The residue was tritureated with Et₂O/iso-hexane to give phenylmethyl [(2R,3S)-3-amino-2-hydroxy-4-phenylbutyl]carbamate hydrochloride (C53) (22.1 g, 83%) as a white solid which was used in the next step without further purification.

Amines 54 and 56-66 (C54 and C56-C66)

Amines 54 and 56-66 were prepared in an analogous manner to that described for Amine 1 (C1), substituting the appropriate BOC-protected amines for tert-butyl [(1S,2R)-1-benzyl-3-(cyclohexylamino)-2-hydroxypropyl]carbamate. In some cases the 4M HCl in dioxane was replaced with 3 equivalents of p-toluene sulphonic acid to give the tosic acid salts as the product.

| Amine | Precursor |
|---|---|
| (2R,3S)-3-Amino-1-{[(6-bromo-2-pyridinyl)methyl]amino}-4-phenyl-2- | H54 |
| butanol (C54) | |
| (2R,3S)-3-Amino-4-phenyl-1-({[1-(2,2,2-trifluoroethyl)-1H-pyrazol-4- | H56 |
| yl]methyl}amino)-2-butanol di-tosylate (C56) | |
| 5-({[(2R,3S)-3-Amino-2-hydroxy-4-phenylbutyl]amino}methyl)-N-methyl- | H57 |
| 3-pyridinecarboxamide di-tosylate (C57) | |
| (2R,3S)-3-Amino-1-[(2,2′-bipyridin-6-ylmethyl)amino]-4-phenyl-2- | H58 |
| butanol di-tosylate (C58) | |
| (2R,3S)-3-Amino-1-{[(6-methyl-2-quinoxalinyl)methyl]amino}-4-phenyl- | H59 |
| 2-butanol di-tosylate (C59) | |
| (2R,3S)-3-Amino-4-phenyl-1-[(3-quinolinylmethyl)amino]-2-butanol di- | H60 |
| tosylate (C60) | |
| (2R,3S)-3-Amino-1-{[(6-methyl-2-pyridinyl)methyl]amino}-4-phenyl-2- | H61 |
| butanol di-tosylate (C61) | |
| (2R,3S)-3-Amino-1-{[(5-ethyl-3-thienyl)methyl]amino}-4-phenyl-2- | H62 |
| butanol di-tosylate (C62) | |
| (2R,3S)-3-Amino-1-{[(5-methyl-2-pyrazinyl)methyl]amino}-4-phenyl-2- | H63 |
| butanol di-hydrochloride (C63) | |
| (2R,3S)-3-Amino-1-{[(3-ethyl-5-isoxazolyl)methyl]amino}-4-phenyl-2- | H64 |
| butanol di-tosylate (C64) | |
| N²-[(2R,3S)-3-Amino-2-hydroxy-4-phenylbutyl]-N¹-cyclohexyl-L- | H65 |
| alaninamide di-hydrochloride (C65) | |
| (2R,3S)-3-Amino-1-[(4,4-difluorocyclohexyl)amino]-4-phenyl-2-butanol | H66 |
| (C66) | |

### PREPARATION OF EXAMPLES

### Example 1

### 7-Ethyl-2-oxo-1,2,3,4-tetrahydro-[1,4]diazepino[3,2,1-hi]indole-9-carboxylic acid [(1S,2R)-1-benzyl-2-hydroxy-3-(3-methoxy-benzylamino)-propyl]-amide (E1)

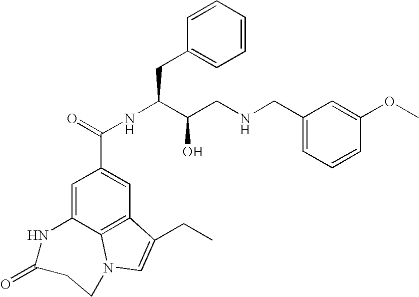

To a solution of 7-ethyl-2-oxo-1,2,3,4-tetrahydro-[1,4]diazepino[3,2,1-hi]indole-9-carboxylic acid (A1) (31 mg, 0.12 mmol, 1 equiv) in DMF (2 ml) and CH₂Cl₂ (8 ml) at room temperature was added (2R,3S)-3-amino-1-(3-methoxy-benzylamino)-4-phenyl-butan-2-ol di-tosylate (C2) (77 mg, 0,12 mmol, 1 equiv), 1-(3-dimethylaminopropyl)-3-ethyl-carbodiimide hydrochloride (28 mg, 0.15 mmol, 1.2 equiv), 1-hydroxybenzotriazole hydrate (22 mg, 0.15 mmol, 1.2 equiv) and 4-ethylmorpholine (34 μl, 0.27 mmol, 2.2 equiv). The resulting mixture was stirred for 4 h then a saturated aqueous NaHCO₃ solution (10 ml) was added. The resulting mixture was vigorously stirred for 20 min. The layers were separated through an hydrophobic frit and the organic phase was concentrated in vacuo. The residue was purified by trituration with Et₂O to yield 7-ethyl-2-oxo-1,2,3,4-tetrahydro-[1,4]diazepino[3,2,1-hi]indole-9-carboxylic acid [(1S,2R)-1-benzyl-2-hydroxy-3-(3-methoxy-benzylamino)-propyl]-amide (E1) as a white solid (43.5 mg, 67%). [M+H]⁺=541.5, RT=2.51 min.

### Examples 2-88 (E2-E88)

Examples 2-88 were obtained in an analogous manner to the procedure described for Example 1 using the appropriate acid and the appropriate amine:

| | | | | | RT |
|---|---|---|---|---|---|
| Example | Structure | Acid | Amine | [M + H]⁺ | (min) |
| 7-Ethyl-N-[(1S,2R)-2-hydroxy-3- ({[3-(methyloxy)phenyl]methyl}amino)-1-(phenylmethyl)propyl]- 3,4-dihydro-1H- [1,2,5]thiadiazepino[3,4,5- hi]indole-9-carboxamide 2,2- dioxide (E2) | 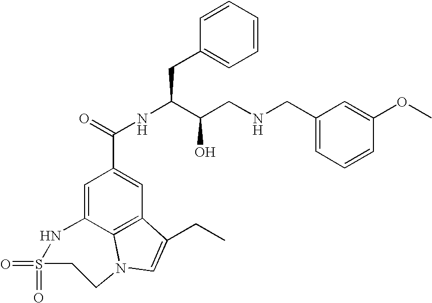 | A2 | C2 | 577.4 | 2.52 |
| 7-Ethyl-N-[(1S,2R)-2-hydroxy-3- ({[3-(methyloxy)phenyl]methyl}amino)-1-(phenylmethyl)propyl]- 1-methyl-3,4-dihydro-1H- [1,2,5]thiadiazepino[3,4,5- hi]indole-9-carboxamide 2,2- dioxide (E3) | 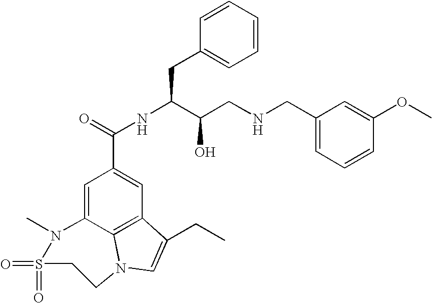 | A3 | C2 | 591.4 | 2.6 |
| 7-Ethyl-N-[(1S,2R)-2-hydroxy-3- ({[3-(methyloxy)phenyl]methyl}amino)-1-(phenylmethyl)propyl]- 1-phenyl-3,4-dihydro-1H- [1,2,5]thiadiazepino[3,4,5- hi]indole-9-carboxamide 2,2- dioxide (E4) | 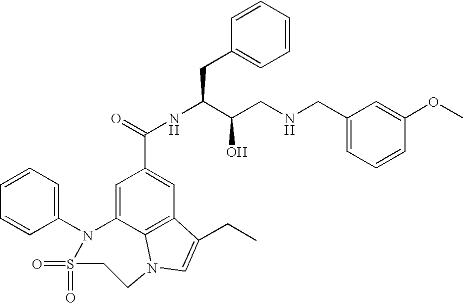 | A4 | C2 | 653.3 | 2.85 |
| 7-Ethyl-N-[(1S,2R)-2-hydroxy-1- (phenylmethyl)-3-({[3- (trifluoromethyl)phenyl]methyl}amino)propyl]-1-methyl-3,4- dihydro-1H-[1,2,5]thiadiazepino [3,4,5-hi]indole-9-carboxamide 2,2-dioxide formate salt (E5) | 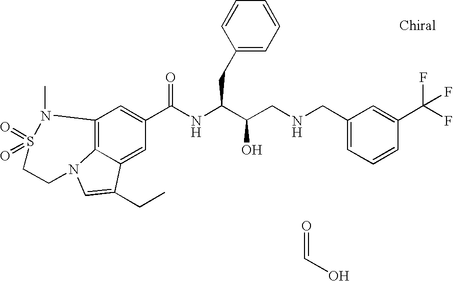 | A3 | C3 | 629.5 | 2.69 |
| 7-Ethyl-N-[(1S,2R)-2-hydroxy-1- (phenylmethyl)-3-({[3- (trifluoromethyl)phenyl]methyl}amino)propyl]-1,3-dimethyl-3,4- dihydro-1H-[1,2,5]thiadiazepino [3,4,5-hi]indole-9-carboxamide 2,2-dioxide formate salt (E6) | 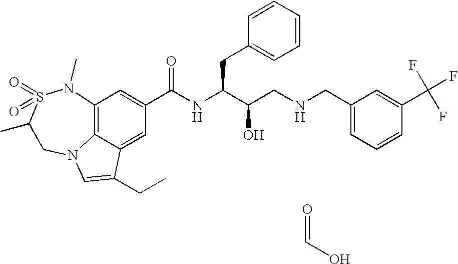 | A5 | C3 | 643.5 | 2.78 |
| N-[(1S,2R)-3-(Cyclohexylamino)- 2-hydroxy-1- (phenylmethyl)propyl]-7-ethyl-1- methyl-3,4-dihydro-1H- [1,2,5]thiadiazepino[3,4,5- hi]indole-9-carboxamide 2,2- dioxide formate salt (E7) | 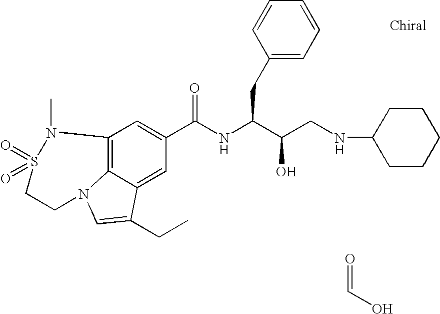 | A3 | C1 | 553.5 | 2.42 |
| N-[(1S,2R)-3-(Cyclohexylamino)- 2-hydroxy-1- (phenylmethyl)propyl]-7-ethyl-1,3- dimethyl-3,4-dihydro-1H- [1,2,5]thiadiazepino[3,4,5- hi]indole-9-carboxamide 2,2- dioxide formate salt (E8) | 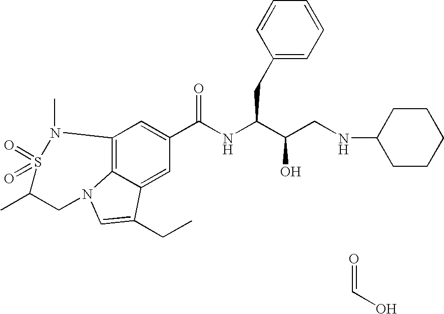 | A5 | C1 | 567.6 | 2.61 |
| 7-Ethyl-N-{(1S,2R)-2-hydroxy-1- (phenylmethyl)-3-[(1,1,5- trimethylhexyl)amino]propyl}-1,3- dimethyl-3,4-dihydro-1H- [1,2,5]thiadiazepino[3,4,5- hi]indole-9-carboxamide 2,2- dioxide formate salt (E9) | 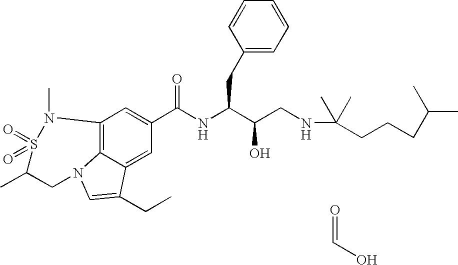 | A5 | C17 | 611.7 | 2.99 |
| 7-Ethyl-N-{(1S,2R)-2-hydroxy-1- (phenylmethyl)-3-[(1,1,5- trimethylhexyl)amino]propyl}-1- methyl-3,4-dihydro-1H- [1,2,5]thiadiazepino[3,4,5- hi]indole-9-carboxamide 2,2- dioxide formate salt (E10) | 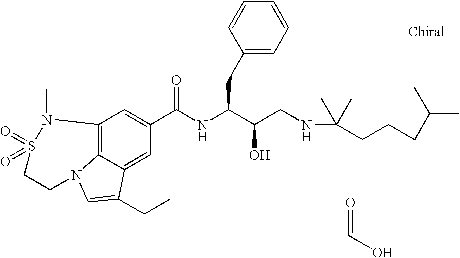 | A3 | C17 | 597.6 | 2.88 |
| 7-Ethyl-N-[(1S,2R)-2-hydroxy-3- ({1-methyl-1-[3- (trifluoromethyl)phenyl]ethyl}amino)-1-(phenylmethyl) propyl]- 1-methyl-3,4-dihydro-1H- [1,2,5]thiadiazepino[3,4,5- hi]indole-9-carboxamide 2,2- dioxide formate salt (E11) | 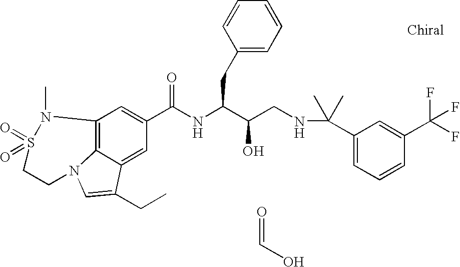 | A3 | C5 | 657.5 | 2.81 |
| 7-Ethyl-N-[(1S,2R)-2-hydroxy-1- (phenylmethyl-3-({[3- (trifluoromethyl)phenyl]methyl}amino)propyl]-1-(phenylmethyl)- 3,4-dihydro-1H- [1,2,5]thiadiazepino[3,4,5- hi]indole-9-carboxamide 2,2- dioxide formate salt (E12) | 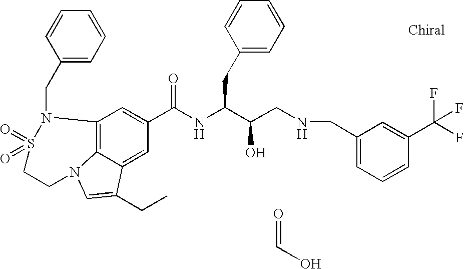 | A6 | C3 | 705.5 | 3.02 |
| 7-Ethyl-N-[(1S,2R)-2-hydroxy-3- ({1-methyl-1-[3- (methyloxy)phenyl]ethyl}amino)- 1-(phenylmethyl)propyl]-1,3- dimethyl-3,4-dihydro-1H- [1,2,5]thiadiazepino[3,4,5- hi]indole-9-carboxamide 2,2- dioxide formate salt (E13) | 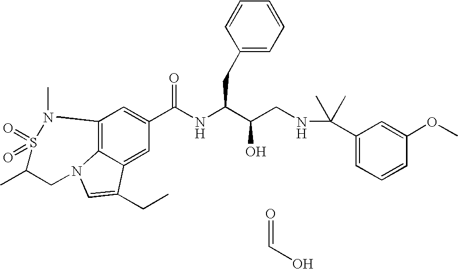 | A5 | C4 | 633.6 | 2.64 |
| 7-Ethyl-N-[(1S,2R)-2-hydroxy-3- ({1-methyl-1-[3- (methyloxy)phenyl]ethyl}amino)- 1-(phenylmethyl)propyl]-1-methyl- 3,4-dihydro-1H- [1,2,5]thiadiazepino[3,4,5- hi]indole-9-carboxamide 2,2- dioxide formate salt (E14) | 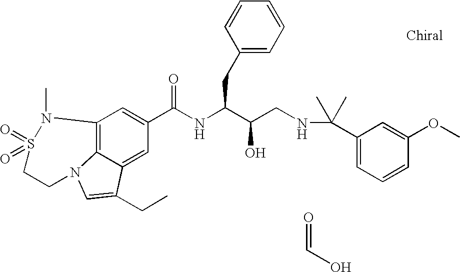 | A3 | C4 | 619.6 | 2.6 |
| 7-Ethyl-N-[(1S,2R)-3-{[(1-ethyl- 1H-pyrazol-4-yl)methyl]amino}-2- hydroxy-1-(phenylmethyl)propyl]- 1-methyl-3,4-dihydro-1H- [1,2,5]thiadiazepino[3,4,5- hi]indole-9-carboxamide 2,2- dioxide formate salt (E15) | 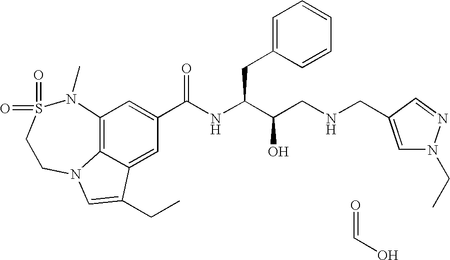 | A3 | C18 | 579.6 | 2.34 |
| 7-Ethyl-N-[(1S,2R)-3-{[(1-ethyl- 1H-pyrazol-4-yl)methyl]amino}-2- hydroxy-1-(phenylmethyl)propyl]- 1,3-dimethyl-3,4-dihydro-1H- [1,2,5]thiadiazepino[3,4,5- hi]indole-9-carboxamide 2,2- dioxide formate salt (E16) | 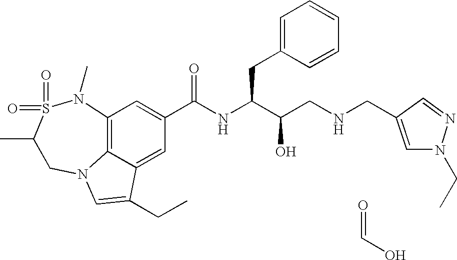 | A5 | C18 | 593.6 | 2.43 |
| 7-Ethyl-N-[(1S,2R)-2-hydroxy-3- [(1-methylethyl)amino]-1- (phenylmethyl)propyl]-1-methyl- 3,4-dihydro-1H- [1,2,5]thiadiazepino[3,4,5- hi]indole-9-carboxamide 2,2- dioxide formate salt (E17) | 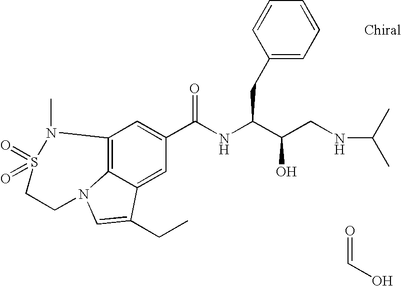 | A3 | C25 | 513.5 | 2.28 |
| 7-Ethyl-N-[(1S,2R)-2-hydroxy-1- (phenylmethyl)-3-(tetrahydro-2H- pyran-4-ylamino)propyl]-1- methyl-3,4-dihydro-1H- [1,2,5]thiadiazepino[3,4,5- hi]indole-9-carboxamide 2,2- dioxide formate salt (E18) | 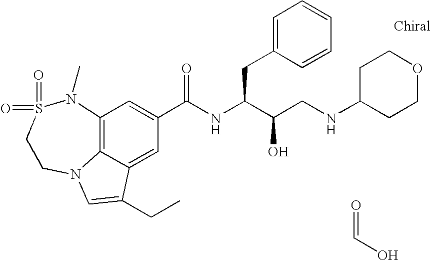 | A3 | C15 | 555.6 | 2.28 |
| N-[(1S,2R)-3-(Cyclopropylamino)- 2-hydroxy-1- (phenylmethyl)propyl]-7-ethyl-1- methyl-3,4-dihydro-1H- [1,2,5]thiadiazepino[3,4,5- hi]indole-9-carboxamide 2,2- dioxide formate salt (E19) | 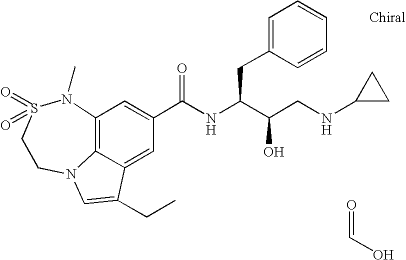 | A3 | C26 | 511.5 | 2.26 |
| 7-Ethyl-N-[(1S,2R)-2-hydroxy-1- (phenylmethyl)-3-(tetrahydro-2H- pyran-4-ylamino)propyl]-1-(1- methylethyl)-3,4-dihydro-1H- [1,2,5]thiadiazepino[3,4,5- hi]indole-9-carboxamide 2,2- dioxide formate salt (E20) | 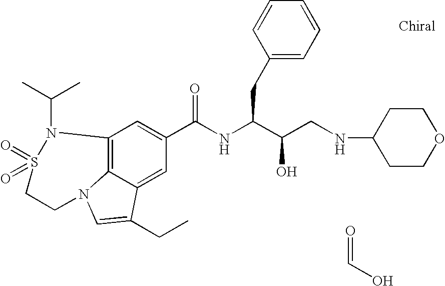 | A7 | C15 | 538.5 | 2.41 |
| 1,7-Diethyl-N-[(1S,2R)-2-hydroxy- 1-(phenylmethyl)-3-(tetrahydro- 2H-pyran-4-ylamino)propyl]-3,4- dihydro-1H- [1,2,5]thiadiazepino[3,4,5- hi]indole-9-carboxamide 2,2- dioxide formate salt (E21) | 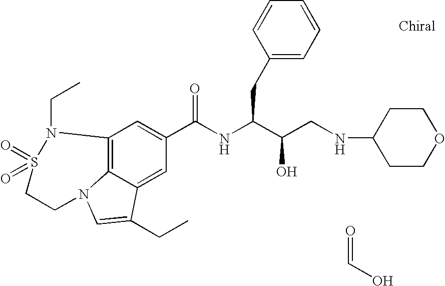 | A8 | C15 | 569.5 | 2.36 |
| N-[(1S,2R)-2-Hydroxy-3-[(1- methylethyl)amino]-1- (phenylmethyl)propyl]-1-methyl-7- (1-methylethyl)-3,4-dihydro-1H- [1,2,5]thiadiazepino[3,4,5- hi]indole-9-carboxamide 2,2- dioxide formate salt (E22) | 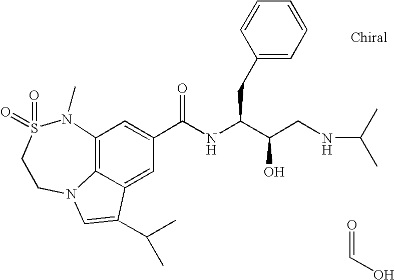 | A9 | C25 | 527.5 | 2.47 |
| N-[(1S,2R)-3-(Cyclohexylamino)- 2-hydroxy-1- (phenylmethyl)propyl]-1-methyl-7- (1-methylethyl)-3,4-dihydro-1H- [1,2,5]thiadiazepino[3,4,5- hi]indole-9-carboxamide 2,2- dioxide formate salt (E23) | 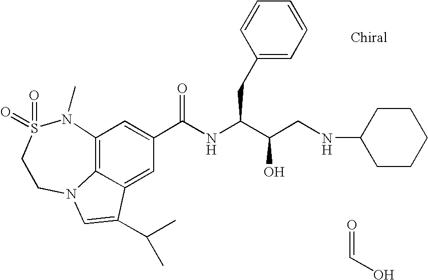 | A9 | C1 | 567.5 | 2.67 |
| N-[(1S,2R)-3-(Cyclopropylamino)- 2-hydroxy-1- (phenylmethyl)propyl]-1-methyl-7- (1-methylethyl-3,4-dihydro-1H- [1,2,5]thiadiazepino[3,4,5- hi]indole-9-carboxamide 2,2- dioxide formate salt (E24) | 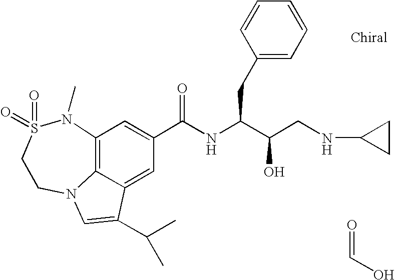 | A9 | C26 | 525.5 | 2.46 |
| N-[(1S,2R)-2-Hydroxy-1- (phenylmethyl)-3-(tetrahydro-2H- pyran-4-ylamino)propyl]-1- methyl-7-(1-methylethyl)-3,4- dihydro-1H- [1,2,5]thiadiazepino[3,4,5- hi]indole-9-carboxamide 2,2- dioxide formate salt (E25) | 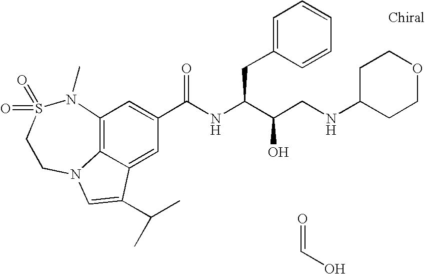 | A9 | C15 | 569.5 | 2.39 |
| N-[(1S,2R)-2-Hydroxy-3-({[3- (methyloxy)phenyl]methyl}amino)-1-(phenylmethyl) propyl]- 1-methyl-7-(1-methylethyl)-3,4- dihydro-1H- [1,2,5]thiadiazepino[3,4,5- hi]indole-9-carboxamide 2,2- dioxide formate salt (E26) | 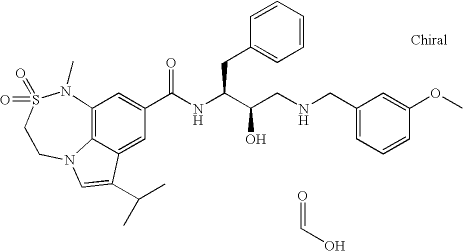 | A9 | C2 | 605.5 | 2.66 |
| N-[(1S,2R)-3-(Cyclohexylamino)- 2-hydroxy-1- (phenylmethyl)propyl]-1,7-diethyl- 3,4-dihydro-1H- [1,2,5]thiadiazepino[3,4,5- hi]indole-9-carboxamide 2,2- dioxide formate salt (E27) | 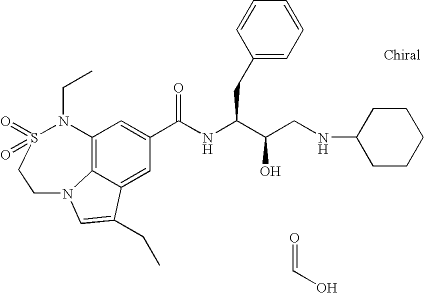 | A8 | C1 | 567.5 | 2.55 |
| 7-Ethyl-N-{(1S,2R)-2-hydroxy-1- (phenylmethyl)-3-[(2,2,2- trifluoroethyl)amino]propyl}-1- methyl-3,4-dihydro-1H- [1,2,5]thiadiazepino[3,4,5- hi]indole-9-carboxamide 2,2- dioxide formate salt (E28) | 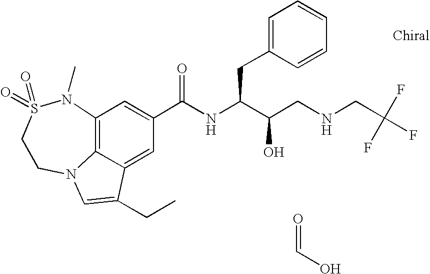 | A3 | C23 | 553.4 | 2.8 |
| 7-Ethyl-N-[(1S,2R)-2-hydroxy-3- [(2,2,3,3,3- pentafluoropropyl)amino]-1- (phenylmethyl)propyl]-1-methyl- 3,4-dihydro-1H- [1,2,5]thiadiazepino[3,4,5- hi]indole-9-carboxamide 2,2- dioxide formate salt (E29) | 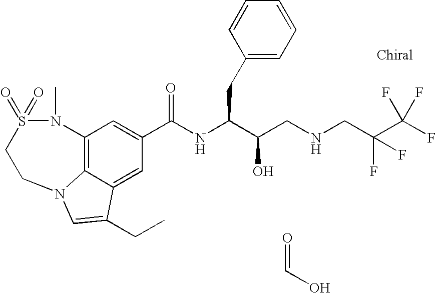 | A3 | C27 | 603.4 | 2.95 |
| N-[(1S,2R)-3- [(Cyclopropylmethyl)amino]-2- hydroxy-1-(phenylmethyl) propyl]- 7-ethyl-1-methyl-3,4-dihydro-1H- [1,2,5]thiadiazepino[3,4,5- hi]indole-9-carboxamide 2,2- dioxide formate salt (E30) | 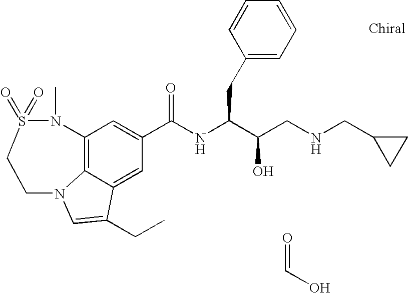 | A3 | C32 | 525.5 | 2.28 |
| N-[(1S,2R)-1-[(3- Chlorophenyl)methyl]-3- (cyclopropylamino)-2- hydroxypropyl]-7-ethyl-1-methyl- 3,4-dihydro-1H- [1,2,5]thiadiazepino[3,4,5- hi]indole-9-carboxamide 2,2- dioxide formate salt (E31) | 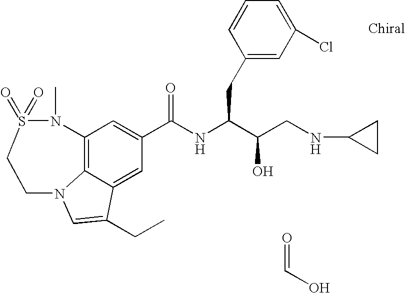 | A3 | C38 | 545.2 | 2.33 |
| N-[(1S,2R)-1-[(3- Chlorophenyl)methyl]-3- (cyclohexylamino)-2- hydroxypropyl]-7-ethyl-1-methyl- 3,4-dihydro-1H- [1,2,5]thiadiazepino[3,4,5- hi]indole-9-carboxamide 2,2- dioxide formate salt (E32) | 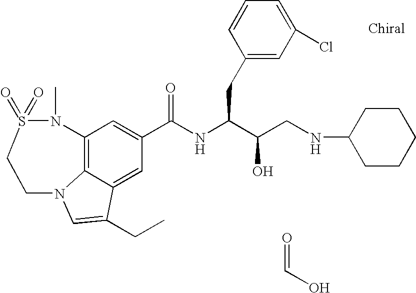 | A3 | C39 | 587.5 | 2.53 |
| N-[(1S,2R)-1-[(3- Chlorophenyl)methyl]-2-hydroxy- 3-(tetrahydro-2H-pyran-4- ylamino)propyl]-7-ethyl-1-methyl- 3,4-dihydro-1H- [1,2,5]thiadiazepino[3,4,5- hi]indole-9-carboxamide 2,2- dioxide formate salt (E33) | 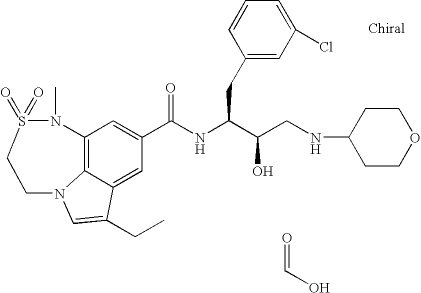 | A3 | C40 | 589.4 | 2.34 |
| N-{(1S,2R)-3- (Cyclopropylamino)-1-[(3- fluorophenyl)methyl]-2- hydroxypropyl}-7-ethyl-1-methyl- 3,4-dihydro-1H- [1,2,5]thiadiazepino[3,4,5- hi]indole-9-carboxamide 2,2- dioxide formate salt (E34) | 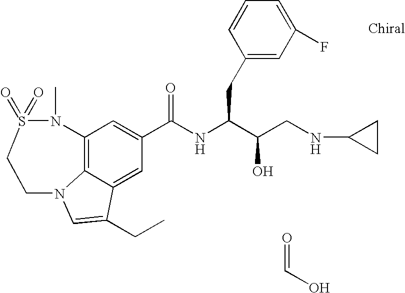 | A3 | C41 | 529.4 | 2.26 |
| 7-Ethyl-N-[(1S,2R)-2-hydroxy-1- (phenylmethyl)-3-(tetrahydro-2H- pyran-4-ylamino)propyl]-1-(2,2,2- trifluoroethyl)-3,4-dihydro-1H- [1,2,5]thiadiazepino[3,4,5- hi]indole-9-carboxamide 2,2- dioxide formate salt (E35) | 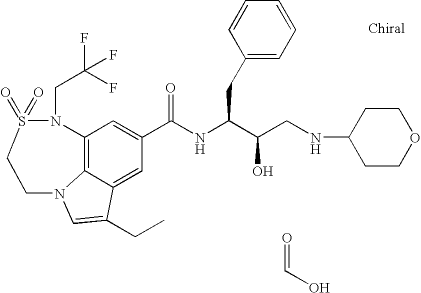 | A10 | C15 | 623.4 | 2.45 |
| N-{(1S,2R)-3-(Cyclohexylamino)- 1-[(3-fluorophenyl)methyl]-2- hydroxypropyl}-7-ethyl-1-methyl- 3,4-dihydro-1H- [1,2,5]thiadiazepino[3,4,5- hi]indole-9-carboxamide 2,2- dioxide formate salt (E36) | 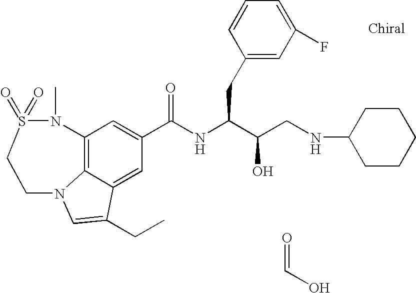 | A3 | C42 | 571.5 | 2.51 |
| 7-Ethyl-N-[(1S,2R)-1-[(3- fluorophenyl)methyl]-2-hydroxy-3- (tetrahydro-2H-pyran-4- ylamino)propyl]-1-methyl-3,4- dihydro-1H- [1,2,5]thiadiazepino[3,4,5- hi]indole-9-carboxamide 2,2- dioxide formate salt (E37) | 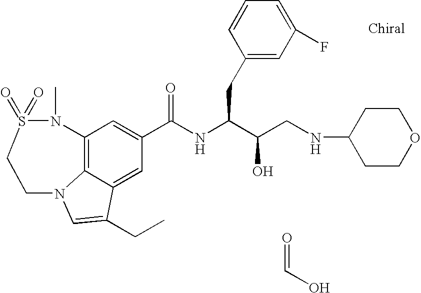 | A3 | C43 | 573.4 | 2.33 |
| N-{(1S,2R)-3-(Cyclohexylamino)- 1-[(3,5-difluorophenyl)methyl]-2- hydroxypropyl}-7-ethyl-1-methyl- 3,4-dihydro-1H- [1,2,5]thiadiazepino[3,4,5- hi]indole-9-carboxamide 2,2- dioxide formate salt (E38) | 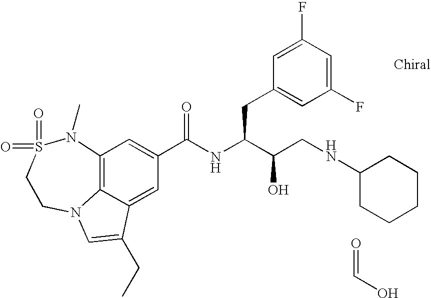 | A3 | C45 | 589.4 | 2.65 |
| N-{(1S,2R)-3- (Cyclopropylamino)-1-[(3,5- difluorophenyl)methyl]-2- hydroxypropyl}-7-ethyl-1-methyl- 3,4-dihydro-1H- [1,2,5]thiadiazepino[3,4,5- hi]indole-9-carboxamide 2,2- dioxide formate salt (E39) | 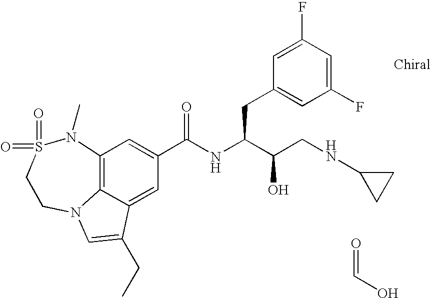 | A3 | C44 | 547.3 | 2.44 |
| N-[(1S,2R)-3-(Cyclobutylamino)- 2-hydroxy-1- (phenylmethyl)propyl]-7-ethyl-1- methyl-3,4-dihydro-1H- [1,2,5]thiadiazepino[3,4,5- hi]indole-9-carboxamide 2,2- dioxide (E40) | 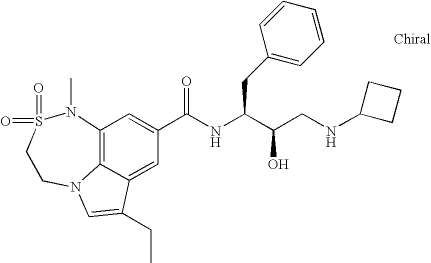 | A3 | C34 | 525.3 | 2.38 |
| 7-Ethyl-N-[(1S,2R)-3-[(2- fluoroethyl)amino]-2-hydroxy-1- (phenylmethyl)propyl]-1-methyl- 3,4-dihydro-1H- [1,2,5]thiadiazepino[3,4,5- hi]indole-9-carboxamide 2,2- dioxide formate salt (E41) | 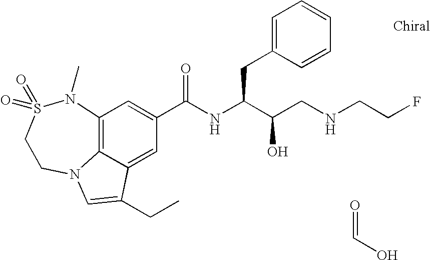 | A3 | C21 | 517.5 | 2.16 |
| N-[(1S,2R)-3-[(2,2- Dimethyltetrahydro-2H-pyran-4- yl)amino]-2-hydroxy-1- (phenylmethyl)propyl]-7-ethyl-1- methyl-3,4-dihydro-1H- [1,2,5]thiadiazepino[3,4,5- hi]indole-9-carboxamide 2,2- dioxide (E42) | 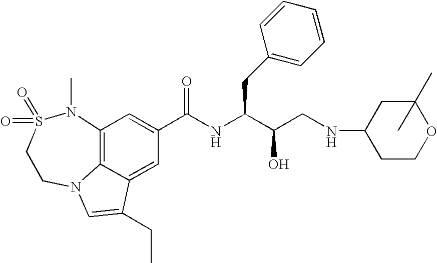 | A3 | C37 | 583.5 | 2.44 |
| N-[(1S,2R)-3-[(1,1- Dimethylethyl)amino]-2-hydroxy- 1-(phenylmethyl)propyl]-7-ethyl- 1-methyl-3,4-dihydro-1H- [1,2,5]thiadiazepino[3,4,5- hi]indole-9-carboxamide 2,2- dioxide (E43) | 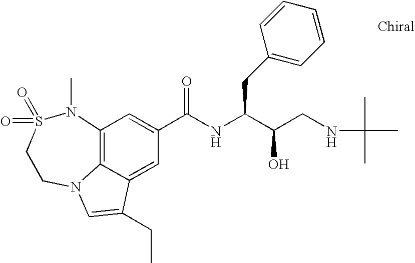 | A3 | C35 | 527.6 | 2.41 |
| N-[(1S,2R)-2-Hydroxy-1- (phenylmethyl)-3-({[3- (trifluoromethyl)phenyl]methyl}am ino)propyl]-1-methyl-7-propyl-3,4- dihydro-1H- [1,2,5]thiadiazepino[3,4,5- hi]indole-9-carboxamide 2,2- dioxide formate salt (E44) | 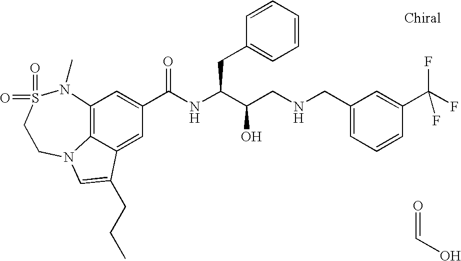 | A11 | C3 | 643.4 | 2.88 |
| N-[(1S,2R)-3-(Cyclohexylamino)- 2-hydroxy-1- (phenylmethyl)propyl]-1-methyl-7- propyl-3,4-dihydro-1H- [1,2,5]thiadiazepino[3,4,5- hi]indole-9-carboxamide 2,2- dioxide formate salt (E45) | 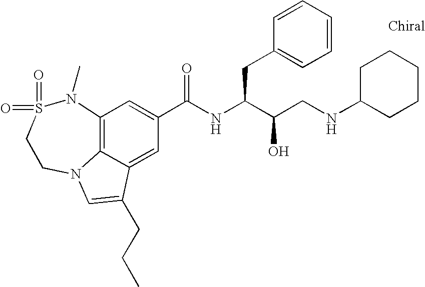 | A11 | C1 | 567.4 | 2.66 |
| N-[(1S,2R)-2-Hydroxy-1- (phenylmethyl)-3-(tetrahydro-2H- pyran-4-ylamino)propyl]-1- methyl-7-propyl-3,4-dihydro-1H- [1,2,5]thiadiazepino[3,4,5- hi]indole-9-carboxamide 2,2- dioxide formate salt (E46) | 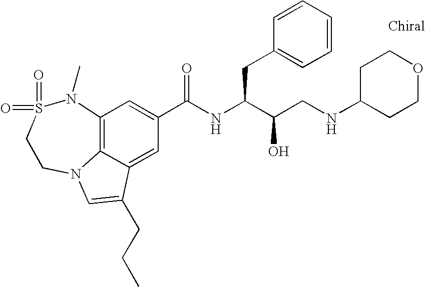 | A11 | C15 | 569.4 | 2.43 |
| N-[(1S,2R)-3-{[(1-Ethyl-1H- pyrazol-4-yl)methyl]amino}-2- hydroxy-1-(phenylmethyl)propyl]- 1-methyl-7-propyl-3,4-dihydro- 1H-[1,2,5]thiadiazepino[3,4,5- hi]indole-9-carboxamide 2,2- dioxide formate salt (E47) | 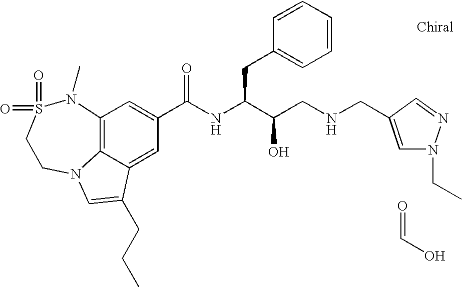 | A11 | C18 | 593.4 | 2.47 |
| 1-Ethyl-N-[(1S,2R)-2-hydroxy-1- (phenylmethyl)-3-({[3- (trifluoromethyl)phenyl]methyl}am ino)propyl]-7-propyl-3,4-dihydro- 1H-[1,2,5]thiadiazepino[3,4,5- hi]indole-9-carboxamide 2,2- dioxide formate salt (E48) | 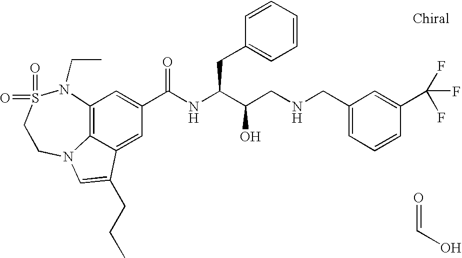 | A12 | C3 | 657.4 | 2.94 |
| N-[(1S,2R)-3-(Cyclohexylamino)- 2-hydroxy-1- (phenylmethyl)propyl]-1-ethyl-7- propyl-3,4-dihydro-1H- [1,2,5]thiadiazepino[3,4,5- hi]indole-9-carboxamide 2,2- dioxide formate salt (E49) | 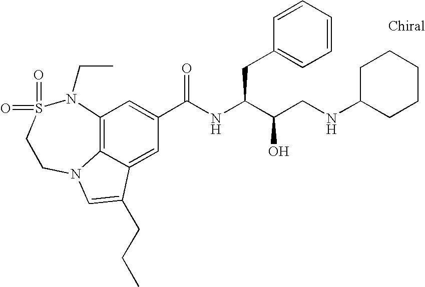 | A12 | C1 | 581.4 | 2.73 |
| 1-Ethyl-N-[(1S,2R)-2-hydroxy-1- (phenylmethyl)-3-(tetrahydro-2H- pyran-4-ylamino)propyl]-7-propyl- 3,4-dihydro-1H- [1,2,5]thiadiazepino[3,4,5- hi]indole-9-carboxamide 2,2- dioxide formate salt (E50) | 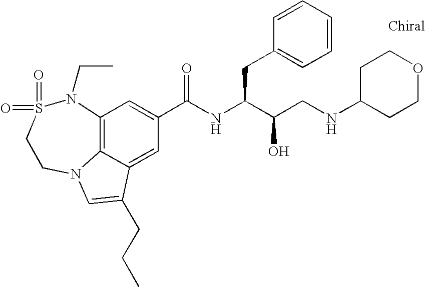 | A12 | C15 | 583.4 | 2.49 |
| 1-Ethyl-N-[(1S,2R)-3-{[(1-ethyl- 1H-pyrazol-4-yl)methyl]amino}-2- hydroxy-1-(phenylmethyl)propyl]- 7-propyl-3,4-dihydro-1H- [1,2,5]thiadiazepino[3,4,5- hi]indole-9-carboxamide 2,2- dioxide formate salt (E51) | 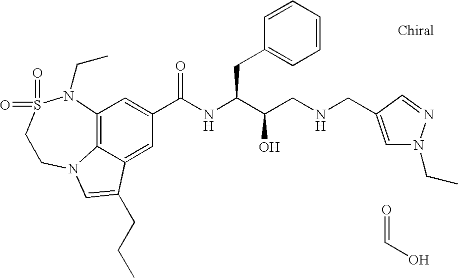 | A12 | C18 | 607.4 | 2.53 |
| N-[(1S,2R)-1-[(3,5- Difluorophenyl)methyl]-2- hydroxy-3-(tetrahydro-2H-pyran- 4-ylamino)propyl]-7-ethyl-1- methyl-3,4-dihydro-1H- [1,2,5]thiadiazepino[3,4,5- hi]indole-9-carboxamide 2,2- dioxide formate salt (E52) | 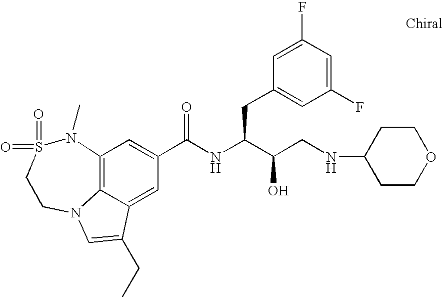 | A3 | C46 | 591.3 | 2.41 |
| 7-Ethyl-N-[(1S,2R)-2-hydroxy-3- {[2-(methyloxy)ethyl]amino}-1- (phenylmethyl)propyl]-1-methyl- 3,4-dihydro-1H- [1,2,5]thiadiazepino[3,4,5- hi]indole-9-carboxamide 2,2- dioxide formate salt (E53) | 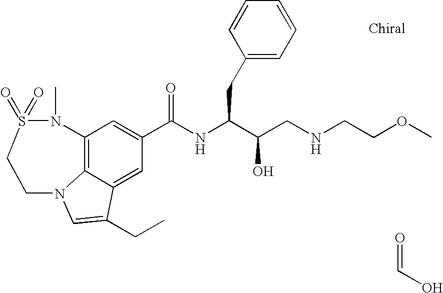 | A3 | C19 | 529.5 | 2.28 |
| 7-Ethyl-N-[(1S,2R)-3- (ethylamino)-2-hydroxy-1- (phenylmethyl)propyl]-1-methyl- 3,4-dihydro-1H- [1,2,5]thiadiazepino[3,4,5- hi]indole-9-carboxamide 2,2- dioxide formate salt (E54) | 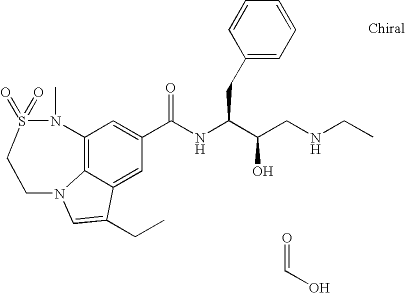 | A3 | C20 | 499.5 | 2.3 |
| 7-Ethyl-N-[(1S,2R)-2-hydroxy-3- {[(1S)-1-methylpropyl]amino}-1- (phenylmethyl)propyl]-1-methyl- 3,4-dihydro-1H- [1,2,5]thiadiazepino[3,4,5- hi]indole-9-carboxamide 2,2- dioxide formate salt (E55) | 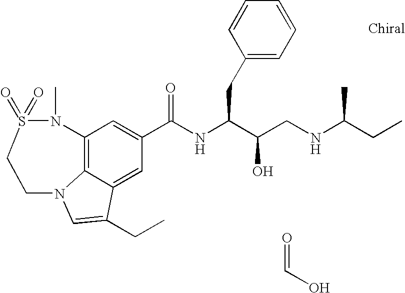 | A3 | C30 | 527.5 | 2.42 |
| N-[(1S,2R)-3-(Butylamino)-2- hydroxy-1-(phenylmethyl)propyl]- 7-ethyl-1-methyl-3,4-dihydro-1H- [1,2,5]thiadiazepino[3,4,5- hi]indole-9-carboxamide 2,2- dioxide formate salt (E56) | 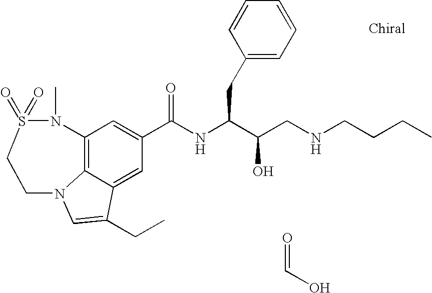 | A3 | C29 | 527.5 | 2.5 |
| 7-Ethyl-N-[(1S,2R)-2-hydroxy-1- (phenylmethyl)-3-(2-propyn-1- ylamino)propyl]-1-methyl-3,4- dihydro-1H- [1,2,5]thiadiazepino[3,4,5- hi]indole-9-carboxamide 2,2- dioxide (E57) | 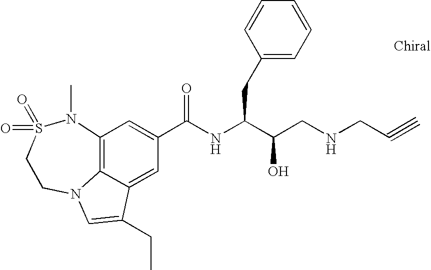 | A3 | C28 | 509.2 | 2.29 |
| N-[(1S,2R)-3-(Cyclopentylamino)- 2-hydroxy-1- (phenylmethyl)propyl]-7-ethyl-1- methyl-3,4-dihydro-1H- [1,2,5]thiadiazepino[3,4,5- hi]indole-9-carboxamide 2,2- dioxide (E58) | 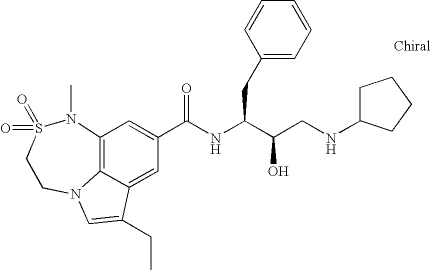 | A3 | C36 | 539.3 | 2.42 |
| 7-Ethyl-N-[(1S,2R)-2-hydroxy-3- [(2-methylpropyl)amino]-1- (phenylmethyl)propyl]-1-methyl- 3,4-dihydro-1H- [1,2,5]thiadiazepino[3,4,5- hi]indole-9-carboxamide 2,2- dioxide (E59) | 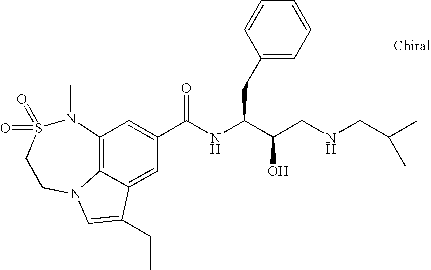 | A3 | C33 | 527.3 | 2.42 |
| 7-Ethyl-N-[(1S,2R)-2-hydroxy-1- (phenylmethyl)-3- (propylamino)propyl]-1-methyl- 3,4-dihydro-1H- [1,2,5]thiadiazepino[3,4,5- hi]indole-9-carboxamide 2,2- dioxide (E60) | 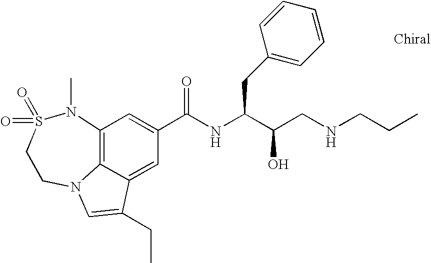 | A3 | C24 | 513.3 | 2.35 |
| 7-Ethyl-N-[(1S,2R)-2-hydroxy-3- {[(1R)-1-methylpropyl]amino}-1- (phenylmethyl)propyl]-1-methyl- 3,4-dihydro-1H- [1,2,5]thiadiazepino[3,4,5- hi]indole-9-carboxamide 2,2- dioxide (E61) | 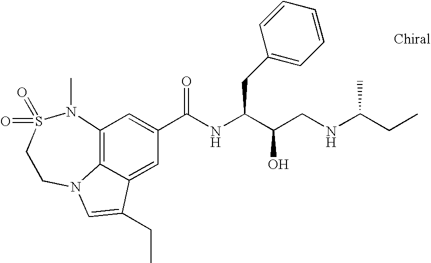 | A3 | C31 | 527.3 | 2.42 |
| N-[(1S,2R)-3-[(2,2- Difluoroethyl)amino]-2-hydroxy-1- (phenylmethyl)propyl]-7-ethyl-1- methyl-3,4-dihydro-1H- [1,2,5]thiadiazepino[3,4,5- hi]indole-9-carboxamide 2,2- dioxide (E62) | 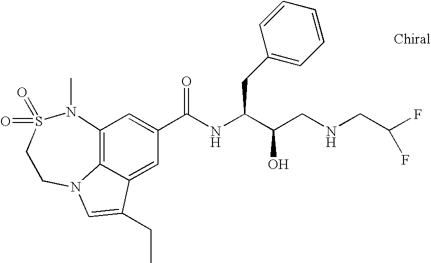 | A3 | C22 | 535.3 | 2.35 |
| 7-Ethyl-N-{(1S,2R)-2-hydroxy-1- (phenylmethyl)-3- [(phenylmethyl)amino]propyl}-1- methyl-3,4-dihydro-1H- [1,2,5]thiadiazepino[3,4,5- hi]indole-9-carboxamide 2,2- dioxide formate salt (E63) | 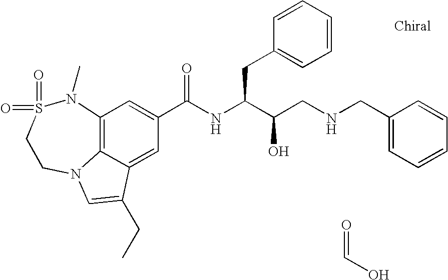 | A3 | C7 | 561.6 | 2.6 |
| 7-Ethyl-N-{(1S,2R)-2-hydroxy-1- (phenylmethyl)-3-[(2- pyridinylmethyl)amino]propyl}-1- methyl-3,4-dihydro-1H- [1,2,5]thiadiazepino[3,4,5- hi]indole-9-carboxamide 2,2- dioxide formate salt (E64) | 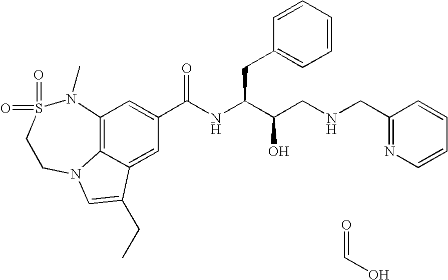 | A3 | C11 | 562.6 | 2.44 |
| 7-Ethyl-N-{(1S,2R)-2-hydroxy-1- (phenylmethyl)-3-[(4- pyridinylmethyl)amino]propyl}-1- methyl-3,4-dihydro-1H- [1,2,5]thiadiazepino[3,4,5- hi]indole-9-carboxamide 2,2- dioxide formate salt (E65) | 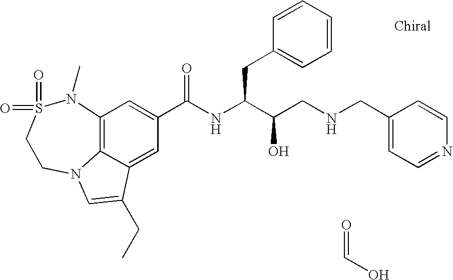 | A3 | C13 | 562.6 | 2.35 |
| 7-Ethyl-N-[(1S,2R)-2-hydroxy-3- [(2-phenylethyl)amino]-1- (phenylmethyl)propyl]-1-methyl- 3,4-dihydro-1H- [1,2,5]thiadiazepino[3,4,5- hi]indole-9-carboxamide 2,2- dioxide formate salt (E66) | 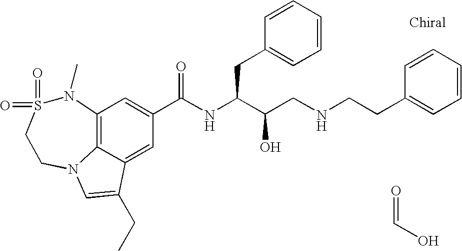 | A3 | C14 | 575.4 | 2.64 |
| 7-Ethyl-N-{(1S,2R)-2-hydroxy-1- (phenylmethyl)-3-[({3- [(trifluoromethyl)oxy]phenyl}methyl)amino]propyl}-1-methyl 3,4-dihydro-1H- [1,2,5]thiadiazepino[3,4,5- hi]indole-9-carboxamide 2,2- dioxide formate salt (E67) | 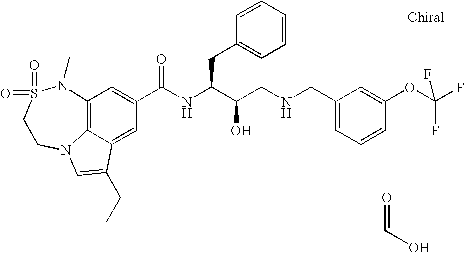 | A3 | C6 | 645.4 | 2.81 |
| 7-Ethyl-N-{(1S,2R)-2-hydroxy-1- (phenylmethyl)-3-[(3- pyridinylmethyl)amino]propyl}-1- methyl-3,4-dihydro-1H- [1,2,5]thiadiazepino[3,4,5- hi]indole-9-carboxamide 2,2- dioxide formate salt (E68) | 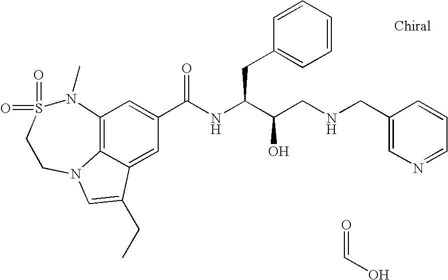 | A3 | C12 | 562.6 | 2.35 |
| 7-Ethyl-N-[(1S,2R)-2-hydroxy-3- {[(2-methylphenyl)methyl]amino}- 1-(phenylmethyl)propyl]-1-methyl- 3,4-dihydro-1H- [1,2,5]thiadiazepino[3,4,5- hi]indole-9-carboxamide 2,2- dioxide formate salt (E69) | 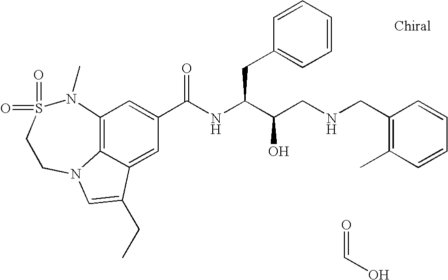 | A3 | C8 | 575.4 | 2.6 |
| 7-Ethyl-N-[(1S,2R)-2-hydroxy-3- {[(3-methylphenyl)methyl]amino}- 1-(phenylmethyl)propyl]-1-methyl- 3,4-dihydro-1H- [1,2,5]thiadiazepino[3,4,5- hi]indole-9-carboxamide 2,2- dioxide formate salt (E70) | 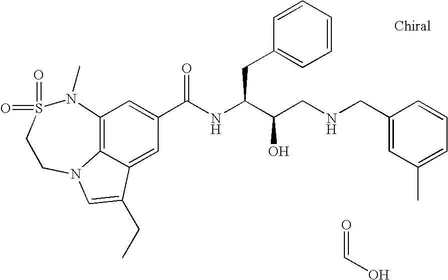 | A3 | C9 | 575.4 | 2.62 |
| 7-Ethyl-N-[(1S,2R)-2-hydroxy-3- {[(4-methylphenyl)methyl]amino}- 1-(phenylmethyl)propyl]-1-methyl- 3,4-dihydro-1H- [1,2,5]thiadiazepino[3,4,5- hi]indole-9-carboxamide 2,2- dioxide formate salt (E71) | 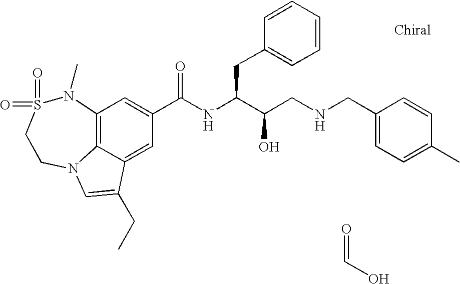 | A3 | C10 | 575.4 | 2.62 |
| N-[(1S,2R)-3-[(1S)-2,3-Dihydro- 1H-inden-1-ylamino]-2-hydroxy-1- (phenylmethyl)propyl]-7-ethyl-1- methyl-3,4-dihydro-1H- [1,2,5]thiadiazepino[3,4,5- hi]indole-9-carboxamide 2,2- dioxide formate salt (E72) | 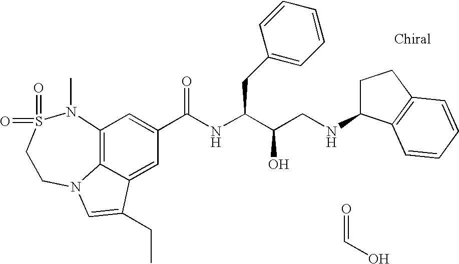 | A3 | C16 | 587.3 | 2.58 |
| 1,1-Dimethylethyl [7-ethyl-9- ({[(1S,2R,)-2-hydroxy-1- (phenylmethyl)-3-({[3- (trifluoromethyl)phenyl]methyl}amino)propyl]amino}carbonyl)- 2,2-dioxido-3,4-dihydro-1H- [1,2,5]thiadiazepino[3,4,5- hi]indol-1-yl]acetate formate salt (E73) | 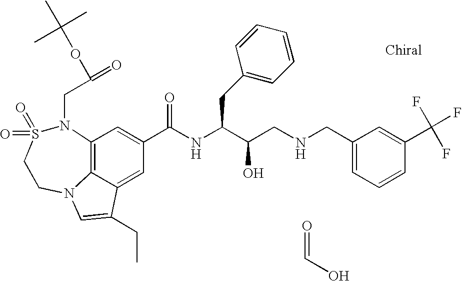 | A13 | C3 | — | 2.7 |
| 7-Ethyl-N-[(1S,2R)-2-hydroxy-1- (phenylmethyl)-3-({[1-(2,2,2- trifluoroethyl)-1H-pyrazol-4- yl]methyl}amino)propyl]-1-methyl- 3,4-dihydro-1H- [1,2,5]thiadiazepino[3,4,5- hi]indole-9-carboxamide 2,2- dioxide formate salt (E74) | 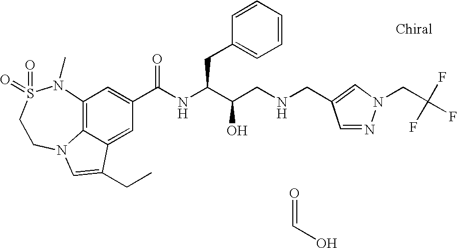 | A3 | C56 | 633.4 | 2.48 |
| 6-Ethyl-N-{(1S,2R)-2-hydroxy-1- (phenylmethyl)-3-[(1,1,5- trimethylhexyl)amino]propyl}-1H- [1,2,5]thiadiazino[3,4,5-hi]indole- 8-carboxamide 2,2-dioxide formate salt (E75) | 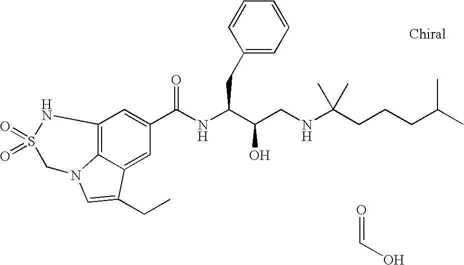 | A14 | C17 | 569.6 | 2.78 |
| N-[(1S,2R)-3-(Cyclohexylamino)- 2-hydroxy-1- (phenylmethyl)propyl]-6-ethyl-1H- [1,2,5]thiadiazino[3,4,5-hi]indole- 8-carboxamide 2,2-dioxide formate salt (E76) | 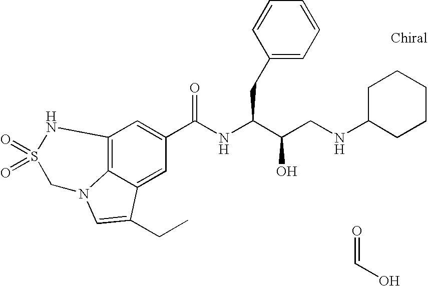 | A14 | C1 | 525.5 | 2.39 |
| 6-Ethyl-N-[(1S,2R)-2-hydroxy-1- (phenylmethyl)-3-({[3- (trifluoromethyl)phenyl]methyl}amino)propyl]-1H- [1,2,5]thiadiazino[3,4,5-hi]indole- 8-carboxamide 2,2-dioxide formate salt (E77) | 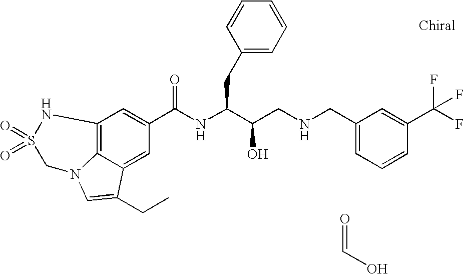 | A14 | C3 | 601.5 | 2.6 |
| 6-Ethyl-N-[(1S,2R)-2-hydroxy-1- (phenylmethyl)-3-({[3- (trifluoromethyl)phenyl]methyl}amino)propyl]-1,3dimethyl-1H- [1,2,5]thiadiazino[3,4,5-hi]indole- 8-carboxamide 2,2-dioxide formate salt (E78) | 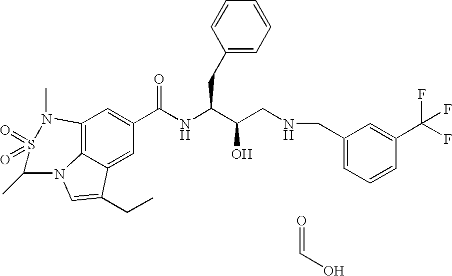 | A15 | C3 | 629.5 | 2.87 |
| 6-Ethyl-N-[(1S,2R)-2-hydroxy-3- ({1-methyl-1-[3- (trifluoromethyl)phenyl]ethyl}amino)-1-(phenylmethyl)propyl]- 1-methyl-1H- [1,2,5]thiadiazino[3,4,5-hi]indole- 8-carboxamide 2,2-dioxide formate salt (E79) | 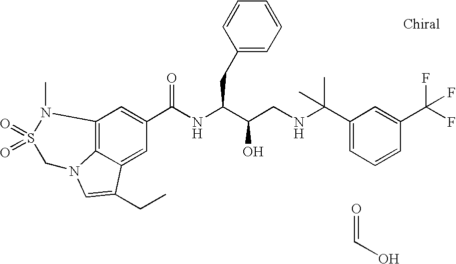 | A16 | C5 | 643.6 | 2.85 |
| N-[(1S,2R)-3-(Cyclohexylamino)- 2-hydroxy-1- (phenylmethyl)propyl]-6-ethyl-1- methyl-1H- [1,2,5]thiadiazino[3,4,5-hi]indole- 8-carboxamide 2,2-dioxide formate salt (E80) | 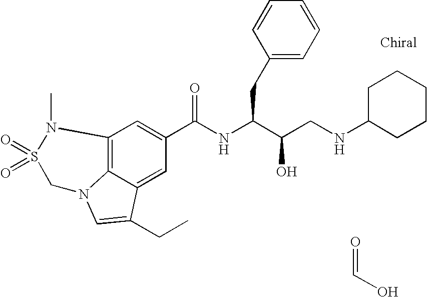 | A16 | C1 | 539.6 | 2.55 |
| 6-Ethyl-N-[(1S,2R)-2-hydroxy-3- ({1-methyl-1-[3- (methyloxy)phenyl]ethyl}amino)- 1-(phenylmethyl)propyl]-1-methyl- 1H-[1,2,5]thiadiazino[3,4,5- hi]indole-8-carboxamide 2,2- dioxide formate salt (E81) | 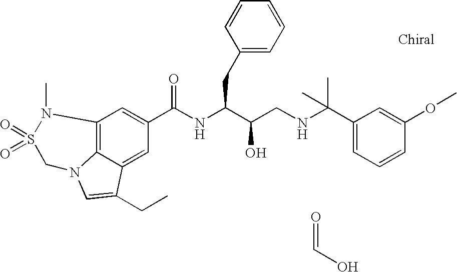 | A16 | C4 | 605.6 | 2.69 |
| 6-Ethyl-N-[(1S,2R)-2-hydroxy-1- (phenylmethyl)-3-(tetrahydro-2H- pyran-4-ylamino)propyl]-1- methyl-1H- [1,2,5]thiadiazino[3,4,5-hi]indole- 8-carboxamide 2,2-dioxide formate salt (E82) | 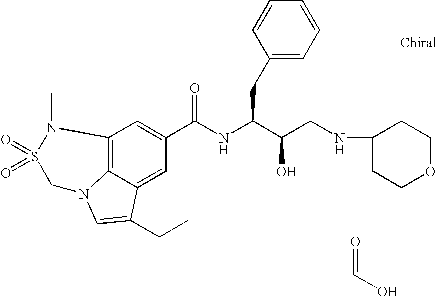 | A16 | C15 | 514.6 | 2.33 |
| 6-Ethyl-N-[(1S,2R)-2-hydroxy-1- (phenylmethyl)-3-({[3- (trifluoromethyl)phenyl]methyl}amino)propyl]-1-methyl-1H- [1,2,5]thiadiazino[3,4,5-hi]indole- 8-carboxamide 2,2-dioxide formate salt (E83) | 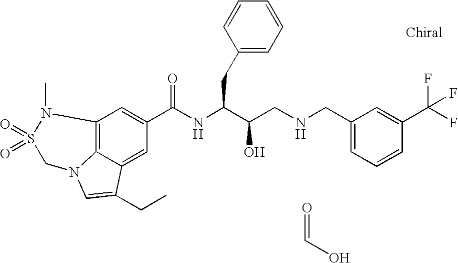 | A16 | C3 | 615.5 | 2.78 |
| 6-Ethyl-N-{(1S,2R)-2-hydroxy-1- (phenylmethyl)-3-[(1,1,5- trimethylhexyl)amino]propyl}-1- methyl-1H- [1,2,5]thiadiazino[3,4,5-hi]indole- 8-carboxamide 2,2-dioxide formate salt (E84) | 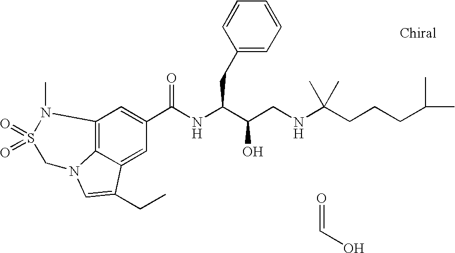 | A16 | C17 | 583.6 | 2.99 |
| 6-Ethyl-N-[(1S,2R)-2-hydroxy-3- [(1-methylethyl)amino]-1- (phenylmethyl)propyl]-1-methyl- 1H-[1,2,5]thiadiazino[3,4,5- hi]indole-8-carboxamide 2,2- dioxide formate salt (E85) | 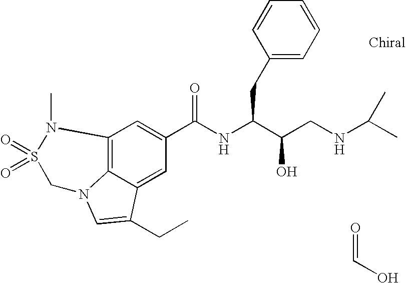 | A16 | C25 | 499.5 | 2.38 |
| 6-Ethyl-N-[(1S,2R)-2-hydroxy-3- ({[3-(methyloxy)phenyl] methyl}amino)-1-(phenylmethyl)propyl]- 1-methyl-1H- [1,2,5]thiadiazino[3,4,5-hi]indole- 8-carboxamide 2,2-dioxide formate salt (E86) | 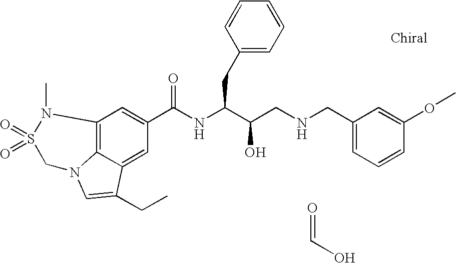 | A16 | C2 | 577.6 | 2.64 |
| 6-Ethyl-N-[(1S,2R)-3-{[(1-ethyl- 1H-pyrazol-4-yl)methyl]amino}-2- hydroxy-1-(phenylmethyl)propyl]- 1-methyl-1H- [1,2,5]thiadiazino[3,4,5-hi]indole- 8-carboxamide 2,2-dioxide formate salt (E87) | 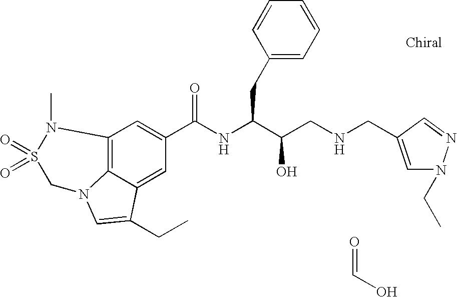 | A16 | C18 | 565.6 | 2.38 |
| 6-Ethyl-N-[(1S,2R)-2-hydroxy-1- (phenylmethyl)-3-(tetrahydro-2H- pyran-4-ylamino)propyl]-1,3,3- trimethyl-1H- [1,2,5]thiadiazino[3,4,5-hi]indole- 8-carboxamide 2,2-dioxide formate salt (E88) | 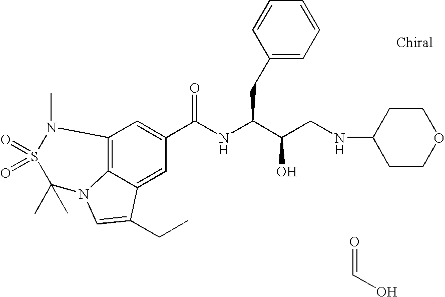 | A17 | C15 | 569.5 | 2.53 |

### Example 89

### N-[(1S,2R)-3-Amino-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide (E89)

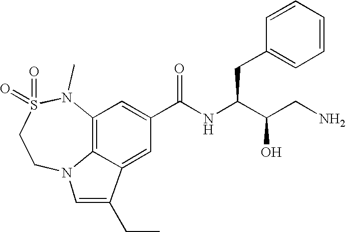

To a solution of phenylmethyl ((2R,3S)-3-{[(7-ethyl-1-methyl-2,2-dioxido-3,4-dihydro-1H-[1,2,5]thiadiazepino[3,4,5-hi]indol-9-yl)carbonyl]amino}-2-hydroxy-4-phenylbutyl)carbamate (D24) (1.35 g, 2.27 mmol, 1 equiv) in AcOEt (20 ml) was added 10% Palladium on charcoal (50% wet, 270 mg, 10% w/w) and the resulting mixture was stirred at room temperature under an atmosphere of hydrogen for 2 h. The catalyst was filtered off through a pad of celite and the solution concentrated in vacuo to give N-[(1S,2R)-3-amino-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide (E89) (900 mg, 90%) as a white foam. [M+H]⁺=471.4, RT=2.14 min.

### Examples 90-94 (E90-E94)

Examples 90-94 were obtained using an analogous procedure to that described in Example 89 from the appropriate precursor indicated in the table below:

| | | | | RT |
|---|---|---|---|---|
| Example | Structure | Precursor | [M + H]⁺ | (min) |
| 7-Ethyl-N-[(1S,2R)-2- hydroxy-3-(methylamino)- 1-(phenylmethyl)propyl]-1- methyl-3,4-dihydro-1H- [1,2,5]thiadiazepino[3,4,5- hi]indole-9-carboxamide 2,2-dioxide formate salt (E90) | 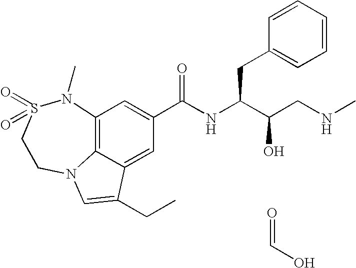 | D22 | 485.5 | 2.24 |
| N-[(1S,2R)-2-Hydroxy-3- (methylamino)-1- (phenylmethyl)propyl]-1- methyl-7-(1-methylethyl)- 3,4-dihydro-1H- [1,2,5]thiadiazepino[3,4,5- hi]indole-9-carboxamide 2,2-dioxide formate salt (E91) | 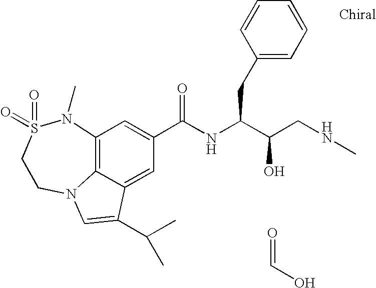 | D23 | 499.5 | 2.37 |
| N-[(1S,2R)-1-[(3- Chlorophenyl)methyl]-2- hydroxy-3- (methylamino)propyl]-7- ethyl-1-methyl-3,4-dihydro- 1H-[1,2,5]thiadiazepino [3,4,5-hi]indole-9- carboxamide 2,2-dioxide formate salt (E92) | 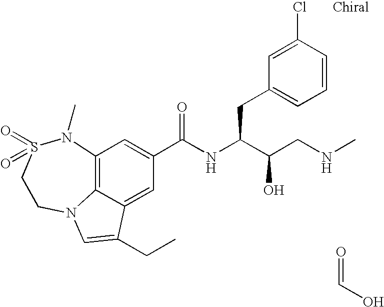 | D20 | 519.4 | 2.30 |
| 7-Ethyl-N-[(1S,2R)-1-[(3- fluorophenyl)methyl]-2- hydroxy-3-(methylamino) propyl]-1-methyl-3,4- dihydro-1H- [1,2,5]thiadiazepino[3,4,5- hi]indole-9-carboxamide 2,2-dioxide formate salt (E93) | 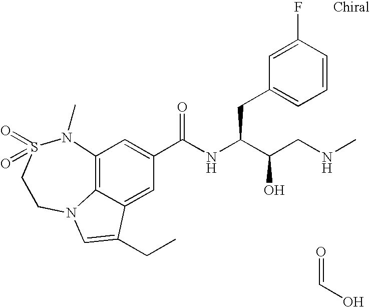 | D21 | 503.5 | 2.26 |
| 6-Ethyl-N-[(1S,2R)-2- hydroxy-3-(methylamino)- 1-(phenylmethyl)propyl]-1- methyl-1H- [1,2,5]thiadiazino[3,4,5- hi]indole-9-carboxamide 2,2-dioxide (E94) | 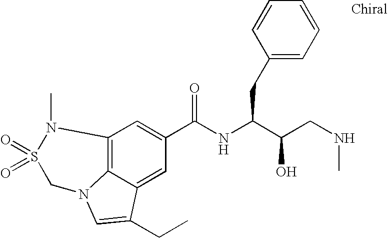 | D25 | 471.5 | 2.29 |

### Example 95

### [7-Ethyl-9-({[(1S,2R)-2-hydroxy-1-(phenylmethyl)-3-({[3-(trifluoromethyl)phenyl]methyl}amino)propyl]amino}carbonyl)-2,2-dioxido-3,4-dihydro-1H-[1,2,5]thiadiazepino[3,4,5-hi]indol-1-yl]acetic acid (E95)

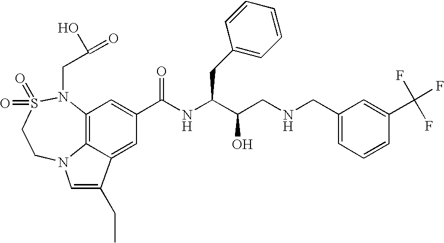

To a solution of 1,1-dimethylethyl [7-ethyl-9-({[(1S,2R)-2-hydroxy-1-(phenylmethyl)-3-({[3-(trifluoromethyl)phenyl]methyl}amino)propyl]amino}carbonyl)-2,2-dioxido-3,4-dihydro-1H-[1,2,5]thiadiazepino[3,4,5-hi]indol-1-yl]acetate formate salt (E73) (10 mg) in CH₂Cl₂ (1 ml) was added TFA (1 ml) and the resulting solution was stirred at room temperature for 1 h then concentrated in vacuo. Trituration of the residue with Et₂O gave [7-ethyl-9-({[(1S,2R)-2-hydroxy-1-(phenylmethyl)-3-({[3-(trifluoromethyl)phenyl]methyl}amino)propyl]amino}carbonyl)-2,2-dioxido-3,4-dihydro-1H-[1,2,5]thiadiazepino[3,4,5-hi]indol-1-yl]acetic acid (E95) (5 mg, 50%) as a white solid. [M+H]+=673.3, RT=2.71 min.

### Examples 96-106 (E96-E106)

Examples 96-106 were obtained in an analogous manner to Example 1 (E1) using the appropriate acid and the appropriate amine indicated in the table below:

| | | | | | RT |
|---|---|---|---|---|---|
| Example | Structure | Acid | Amine | [M + H]⁺ | (min) |
| N-[(1S,2R)-3-{[(6-Bromo-2- pyridinyl)methyl]amino}-2- hydroxy-1- (phenylmethyl)propyl]-7- ethyl-1-methyl-3,4-dihydro- 1H-[1,2,5]thiadiazepino[3,4,5- hi]indole-9-carboxamide 2,2- dioxide formate salt (E96) | 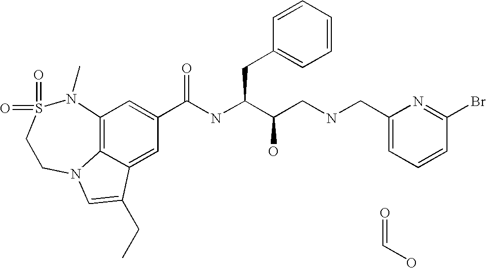 | A3 | C54 | 640.4 | 2.64 |
| 7-Ethyl-N-[(1S,2R)-2 hydroxy-3-[({5- [(methylamino)carbonyl]-3- pyridinyl}methyl)amino]-1- (phenylmethyl)propyl]-1- methyl-3,4-dihydro-1H- [1,2,5]thiadiazepino[3,4,5- hi]indole-9-carboxamide 2,2- dioxide formate salt (E97) | 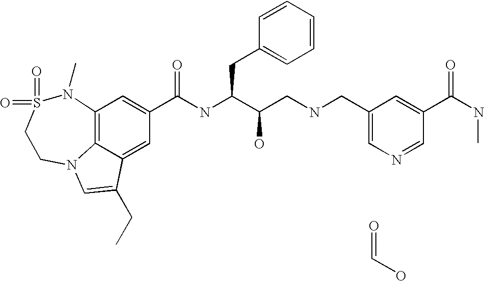 | A3 | C57 | 619.4 | 2.31 |
| N-[(1S,2R)-3-[(2,2′-Bipyridin- 6-ylmethyl)amino]-2- hydroxy-1- (phenylmethyl)propyl]-7- ethyl-1-methyl-3,4-dihydro- 1H-[1,2,5]thiadiazepino[3,4,5- hi]indole-9-carboxamide 2,2- dioxide formate salt (E98) | 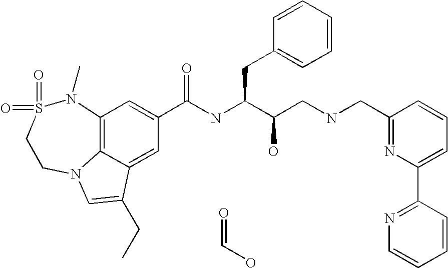 | A3 | C58 | 639.5 | 2.59 |
| 7-Ethyl-N-[(1S,2R)-2- hydroxy-3-{[(6-methyl-2- quinoxalinyl)methyl]amino}- 1-(phenylmethyl)propyl]-1- methyl-3,4-dihydro-1H- [1,2,5]thiadiazepino[3,4,5- hi]indole-9-carboxamide 2,2- dioxide formate salt (E99) | 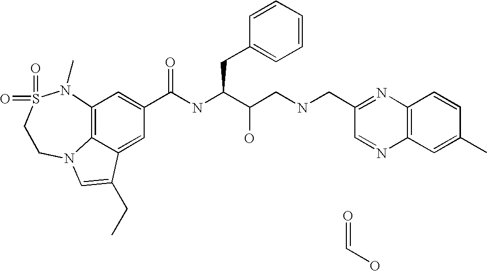 | A3 | C59 | 627.5 | 2.61 |
| 7-Ethyl-N-{(1S,2R)-2- hydroxy-1-(phenylmethyl)-3- [(3-quinolinylmethyl)amino]propyl}-1-methyl-3,4-dihydro- 1H-[1,2,5]thiadiazepino[3,4,5- hi]-indole-9-carboxamide 2,2- dioxide formate salt (E100) | 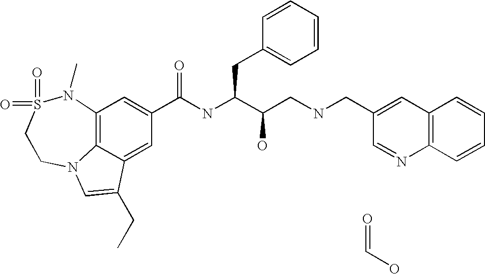 | A3 | C60 | 612.5 | 2.58 |
| 7-Ethyl-N-[(1S,2R)-2- hydroxy-3-{[(6-methyl-2- pyridinyl)methyl]amino}-1- (phenylmethyl)propyl]-1- methyl-3,4-dihydro-1H- [1,2,5]thiadiazepino[3,4,5- hi]indole-9-carboxamide 2,2- dioxide formate salt (E101) | 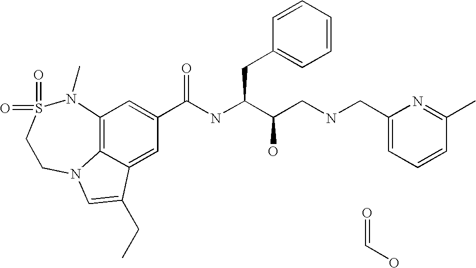 | A3 | C61 | 576.5 | 2.54 |
| 7-Ethyl-N-[(1S,2R)-3-{[(5- ethyl-3- thienyl)methyl]amino}-2- hydroxy-1- (phenylmethyl)propyl]-1- methyl-3,4-dihydro-1H- [1,2,5]thiadiazepino[3,4,5- hi]indole-9-carboxamide 2,2- dioxide formate salt (E102) | 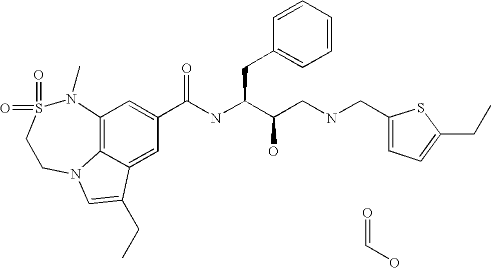 | A3 | C62 | 595.2 | 2.79 |
| 7-Ethyl-N-[(1S,2R)-2- hydroxy-3-{[(5-methyl-2- pyrazinyl)methyl]amino}-1- (phenylmethyl)propyl]-1- methyl-3,4-dihydro-1H- [1,2,5]thiadiazepino[3,4,5- hi]indole-9-carboxamide 2,2- dioxide formate salt (E103) | 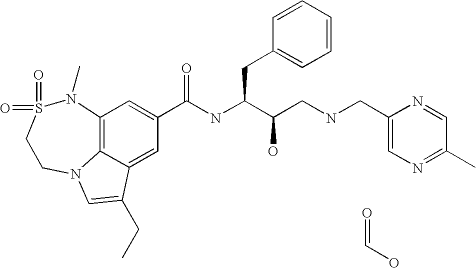 | A3 | C63 | 577.5 | 2.41 |
| 7-Ethyl-N-[(1S,2R)-3-{[(3- ethyl-5- isoxazolyl)methyl]amino}-2- hydroxy-1- (phenylmethyl)propyl]-1- methyl-3,4-dihydro-1H- [1,2,5]thiadiazepino[3,4,5- hi]indole-9-carboxamide 2,2- dioxide (E104) | 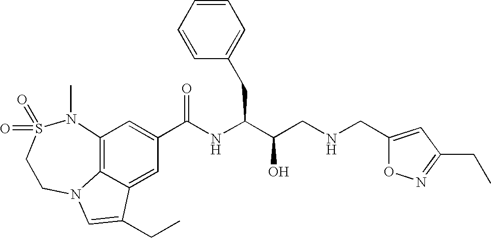 | A3 | C64 | 580.5 | 2.57 |
| N-[(1S,2R)-3-{[(1S)-2- (Cyclohexylamino)-1-methyl- 2-oxoethyl]amino}-2- hydroxy-1- (phenylmethyl)propyl]-7- ethyl-1-methyl-3,4-dihydro- 1H- [1,2,5]thiadiazepino[3,4,5- hi]indole-9-carboxamide 2,2- dioxide (E105) | 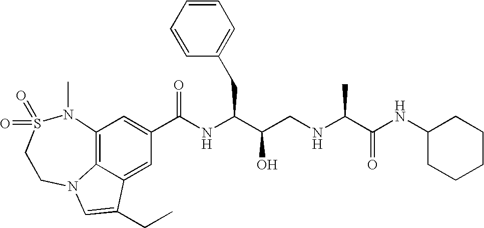 | A3 | C65 | 624.5 | 2.65 |
| N-[(1S,2R)-3-[(4,4- Difluorocyclohexyl)amino]-2- hydroxy-1- (phenylmethyl)propyl]-7- ethyl-1-methyl-3,4-dihydro- 1H- [1,2,5]thiadiazepino[3,4,5- hi]indole-9-carboxamide 2,2- dioxide formate salt (E106) | 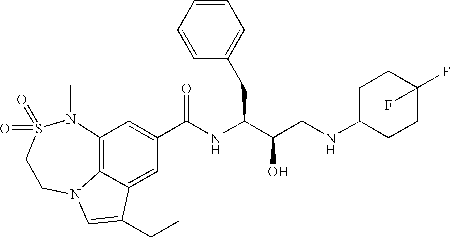 | A3 | C66 | 589.5 | 2.52 |

Compounds of the invention may be tested for in vitro biological activity in accordance with the following assays:

(I) Asp-2 Inhibitory Assay

For each compound being assayed, in a 384 well plate, is added:

a) 1 μl of a DMSO solution of the test compound (IC₅₀ curve uses ten 1 in 2 serial dilutions from 500 μM).

b) 10 μl of substrate (FAM-[SEVNLDAEFK]-TAMRA) solution in buffer. This is prepared by diluting 2 ml of a 2 mM DMSO solution of the substrate into 400 ml of buffer (100 mM Sodium acetate pH=4.5, 1 l Milli-Q water, 0.06% Triton X-100 (0.5 ml/l), pH adjusted to 4.5 using glacial acetic acid). Aminomethyl fluorescein (FAM) and tetramethyl rhodamine (TAMRA) are fluorescent molecules which co-operate to emit fluorescence at 535 nm upon cleavage of the SEVNLDAEFK peptide.

c) 10 μl enzyme solution. This is prepared by diluting 16 ml of a 500 nM enzyme solution into 384 ml of buffer (prepared as above).

Blank wells (enzyme solution replaced by buffer) are included as controls on each plate. Wells are incubated for 1 h at room temperature and fluorescence read using a Tecan Ultra Fluorimeter/Spectrophotometer (485 nm excitation, 535 nm emission).

(II) Cathepsin D Inhibitory Assay

For each compound being assayed, in a 384 well plate, is added:

a) 1 μl of a DMSO solution of the test compound (IC₅₀ curve uses ten 1 in 2 serial dilutions from 500 μM).

b) 10 μl of substrate (FAM-[SEVNLDAEFK]-TAMRA) solution in buffer. This is prepared by diluting 2 ml of a 2 mM DMSO solution of the substrate into 400 ml of buffer (100 mM Sodium acetate pH=4.5, 1 l Milli-Q water, 0.06% Triton X-100 (0.5 ml/l), pH adjusted to 4.5 using glacial acetic acid).

c) 10 μl enzyme solution. This is prepared by diluting 1.6 ml of a 200 unit/ml (in 10 mM HCl) enzyme solution into 398.4 ml of buffer (prepared as above).

Blank wells (enzyme solution replaced by buffer) are included as controls on each plate. Wells are incubated for I h at room temperature and fluorescence read using a Tecan Ultra Fluorimeter/Spectrophotometer (485 nm excitation, 535 nm emission).

Pharmacological Data

The compounds of E1-E106 were tested in the Asp-2 inhibitory assay and exhibited inhibition <10 μM. More particularly, the compounds of Examples E3-E7, E9-E11, E13, E15-E16, E21, E27, E32, E36, E37-E39, E44, E47-E48, E51, E67, E70, E72, E74, E78-E79, E83, E86, E97, E102, E104 and E105-E106 exhibited inhibition <1 μM in the Asp-2 inhibitory assay. Most particularly, the compounds of Examples E3, E5, E15-E16, E39, E47, E51, E67, E70, E74, E97, E102, E104 and E105 were tested in the Asp-2 inhibitory assay and the Cathepsin D inhibitory assay and exhibited inhibition <1 μM in the Asp-2 inhibitory assay and >100 fold selectivity for Asp2 over CatD.

Abbreviations

DMF dimethylformamide

DMSO dimethylsulfoxide

DMAP dimethylaminophenol

DABCO 1,4diazabicyclo [2.2.2] octane

DME dimethyl ether

THF tetrahydrofuran

HOBT N-hydroxybenzotriazole

FAM carboxyfluorescein

TAMRA carboxytetramethylrhodamine

[ ] single amino acid letter code relating to peptide sequence

What is claimed is:

1-8. (canceled)

9. A compound of formula (I): 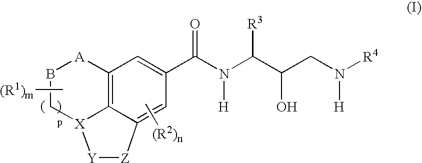
wherein
R¹ and R² independently represent C₁₋₃ alkyl, C₂₋₄ alkenyl, halogen, C₁₋₃ alkoxy, amino, cyano, or hydroxy;
m and n independently represent 0, 1, or 2;
p represents 1 or 2;
A-B represents —NR⁵—SO₂— or —NR⁵—CO—;
R⁵ represents hydrogen, C₁₋₆ alkyl, C₃₋₆ alkenyl, C₃₋₆ alkynyl, C₃₋₈ cycloalkyl, aryl, heteroaryl, arylC₁₋₆ alkyl-, heteroarylC₁₋₆ alkyl-, arylC₃₋₈ cycloalkyl-, or heteroarylC₃₋₈ cycloalkyl-;
X—Y-Z represents —N—CR⁸═CR⁹—;
R⁸ represents hydrogen, C₁₋₆ alkyl, or C₃₋₈ cycloalkyl;
R⁹ represents hydrogen, C₁₋₆ alkyl, C₃₋₈ cycloalkyl, aryl, heteroaryl, arylC₁₋₆ alkyl-, heteroarylC₁₋₆ alkyl-, arylC₃₋₈ cycloalkyl-, heteroarylC₃₋₈ cycloalkyl-, —COOR¹⁰, —OR¹⁰, —CONR¹⁰R¹¹, —SO₂NR¹⁰R¹¹, —COC₁₋₆ alkyl, or —SO₂C₁₋₆ alkyl (wherein R¹⁰ and R¹¹ independently represent hydrogen, C₁₋₆ alkyl, or C₃₋₈ cycloalkyl);
R³ represents optionally substituted C₁₋₆ alkyl, C₂₋₆ alkenyl, C₂₋₆ alkynyl, —C₁₋₆ alkyl-C₃₋₈ cycloalkyl, —C₁₋₆ alkyl-aryl, —C₁₋₆ alkyl-heteroaryl, or —C₁₋₆ alkyl-heterocyclyl;
R⁴ represents hydrogen, optionally substituted C₁₋₁₀ alkyl, C₂₋₆ alkynyl, —C₃₋₈ cycloalkyl, —C₃₋₈ cycloalkenyl, aryl, heteroaryl, heterocyclyl, —C₁₋₆ alkyl-C₃₋₈ cycloalkyl, —C₃₋₈ cycloalkyl-aryl, -heterocyclyl-aryl, —C₁₋₆ alkyl-aryl-heteroaryl, —C(R^{a}R^{b})—CONH—C₁₋₆ alkyl, —C(R^{a}R^{b})—CONH—C₃₋₈ cycloalkyl, —C₁₋₆ alkyl-S—C₁₋₆ alkyl, —C₁₋₆ alkyl-NR^{c}R^{d}, —C(R^{a}R^{b})—C₁₋₆ alkyl, —C(R^{a}R^{b})-aryl, —C(R^{a}R^{b})-heteroaryl, —C(R^{a}R^{b})-herteroaryl-heteroaryl, —C(R^{a}R^{b})—C₁₋₆ alkyl-aryl, —C(R^{a}R^{b})—C₁₋₆ alkyl-heteroaryl, —C(R^{a}R^{b})—C₁₋₆ alkyl-heterocyclyl, —C₁₋₆ alkyl-O—C₁₋₆ alkyl-aryl, —C₁₋₆ alkyl-O—C₁₋₆ alkyl-heteroaryl, or —C₁₋₆ alkyl-O—C₁₋₆ alkyl-heterocyclyl;
R^{a} and R^{b} independently represent hydrogen, C₁₋₆ alkyl, C₂₋₆ alkenyl, C₂₋₆ alkynyl, or C₃₋₈ cycloalkyl, or R^{a} and R^{b} together with the carbon atom to which they are attached may form a C₃₋₈ cycloalkyl or heterocyclyl group;
R^{c} and R^{d} independently represent hydrogen, C₁₋₆ alkyl, C₂₋₆ alkenyl, C₂₋₆ alkynyl, C₃₋₈ cycloalkyl, or R^{c} and R^{d} together with the nitrogen atom to which they are attached may form a nitrogen containing heterocyclyl group;
wherein said aryl, heteroaryl, or heterocyclyl groups of R³—R⁵, R⁹ and R^{a}—R^{d} may be optionally substituted by one or more C₁₋₆ alkyl, halogen, haloC₁₋₆ alkyl, haloC₁₋₆ alkoxy, oxo, C₁₋₆ alkoxy, C₂₋₆ alkynyl, C₂₋₆ alkenyl, amino, cyano, nitro, —NR²²COR²³, —CONR²²R²³—SO₂R²², —SO₂NR²²R²³,—COOR²², —C₁₋₆ alkyl-NR²²R²³ (wherein R²² and R²³ independently represent hydrogen, C₁₋₆ alkyl or C₃₋₈ cycloalkyl), —C₁₋₆ alkyl-O—C₁₋₆ alkyl, —C₁₋₆ alkanoyl, or hydroxy groups;
and wherein said alkyl and cycloalkyl groups of R¹—R⁵, R⁸—R¹¹, R²²—R²³ and R^{a}—R^{d} may be optionally substituted by one or more halogen, C₁₋₆ alkyl, C₁₋₆ alkoxy, C₁₋₆ alkylamino, amino, cyano, hydroxy, carboxy, or —COOC₁₋₆ alkyl groups; or a pharmaceutically acceptable salt or solvate thereof.

10. A compound according to claim 9, wherein A-B represents —NR⁵—SO₂—.

11. A compound according to claim 9, wherein R⁵ represents:
hydrogen;
C₁₋₆ alkyl optionally substituted by one or more halogen atoms, carboxy or —COOC₁₋₆ alkyl groups;
aryl; or
arylC₁₋₆ alkyl-.

12. A compound according to claim 9, wherein m and n represent 0.

13. A compound according to claim 9, wherein p represents 2.

14. A compound according to claim 9, wherein R⁸ represents hydrogen and wherein R⁹ represents hydrogen or C₁₋₆ alkyl.

15. A compound according to claim 9, wherein R³ represents —C₁₋₆ alkyl-aryl optionally substituted by one or two halogen atoms.

16. A compound according to claim 9, wherein R⁴ represents
-hydrogen;
—C₁₋₁₀ alkyl optionally substituted by one or more halogen or C₁₋₆ alkoxy groups;
C₂₋₆ alkynyl;
—C₃₋₈ cycloalkyl optionally substituted by one or more halogen atoms or C₁₋₆ alkyl groups;
—C₁₋₆ alkyl-C₃₋₈ cycloalkyl;
aryl;
-heterocyclyl;
—C(R^{a}R^{b})-aryl optionally substituted by one or more halogen, cyano, nitro, haloC₁₋₆ alkyl, haloC₁₋₆ alkoxy, C₁₋₆ alkyl or C₁₋₆ alkoxy, C₂₋₆ alkynyl, C₂₋₆ alkenyl, amino, —NR²²COR²³, —CONR²²R²³—SO₂R²², —SO₂NR²²R²³, —COOR², C₁₋₆ alkyl-NR²²R²³, —C₁₋₆ alkanoyl, or hydroxy groups;
—C(R^{a}R^{b})-heteroaryl optionally substituted by one or more C₁₋₆ alkyl, halogen, haloC₁₋₆ alkyl, or —CONR²²R²³ groups;
—C(R^{a}R^{b})-heteroaryl-heteroaryl;
—C(R^{a}R^{b})—C₁₋₆ alkyl-aryl;
—C(R^{a}R^{b})—CONH—C₃₋₈ cycloalkyl; or
—C₃₋₈ cycloalkyl-aryl.

17. A compound according to claim 16, wherein R⁴ represents:
—C₃₋₈ cycloalkyl optionally substituted by one or more halogen atoms;
-heterocyclyl;
—C(R^{a}R^{b})-aryl optionally substituted by one or more haloC₁₋₆ alkyl, haloC₁₋₆ alkoxy, C₁₋₆ alkyl, or C₁₋₆ alkoxy groups;
—C(R^{a}R^{b})-heteroaryl optionally substituted by one or more C₁₋₆ alkyl, haloC₁₋₆ alkyl, or —CONR²²R²³ groups; or
—C(R^{a}R^{b})—CONH—C₃₋₈ cycloalkyl.

18. A compound according to claim 9, wherein R^{a} and R^{b} independently represent hydrogen or methyl, or R^{a} and R^{b} together with the carbon atom to which they are attached form a cyclopropyl or cyclohexyl group.

19. A compound according to claim 9 which is:
7-Ethyl-2-oxo-1,2,3,4-tetrahydro-[1,4]diazepino[3,2,1-hi]indole-9-carboxylic acid [(1S,2R)-1-benzyl-2-hydroxy-3-(3-methoxy-benzylamino)-propyl]-amide;
7-Ethyl-N-[(1S,2R)-2-hydroxy-3-({[3-(methyloxy)phenyl]methyl}amino)-1-(phenylmethyl)propyl]-3,4-dihydro-1H-[1,2,5]thiadiazepino[3,4,5-hi]indole-9-carboxamide 2,2-dioxide;
7-Ethyl-N-[(1S,2R)-2-hydroxy-3-({[3-(methyloxy)phenyl]methyl}amino)-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-({[3-(methyloxy)phenyl]methyl}amino)-1-(phenylmethyl)propyl]-1-phenyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-({[3-(trifluoromethyl)phenyl]methyl}amino)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-({[3-(trifluoromethyl)phenyl]methyl}amino)propyl]-1,3-dimethyl-3,4-dihydro-1H-[1,2,5]thiadiazepino [3,4,5-hi]indole-9-carboxamide 2,2-dioxide;
N-[(1S,2R)-3-(Cyclohexylamino)-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
N-[(1S,2R)-3-(Cyclohexylamino)-2-hydroxy-1-(phenylmethyl)propyl]-7-ethyl-1,3-dimethyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(1,1,5-trimethylhexyl)amino]propyl}-1,3-dimethyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(1,1,5-trimethylhexyl)amino]propyl}-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-({1-methyl-1-[3-(trifluoromethyl)phenyl]ethyl}amino)-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-({[3-(trifluoromethyl)phenyl]methyl}amino)propyl]-1-(phenylmethyl)-3,4-dihydro-1H-[1,2,5]thiadiazepino[3,4,5-hi]indole-9-carboxamide 2,2-dioxide;
7-Ethyl-N-[(1S,2R)-2-hydroxy-3-({1-methyl-1-[3-(methyloxy)phenyl]ethyl}amino)-1-(phenylmethyl)propyl]-1,3-dimethyl-3,4-dihydro-1H-[1,2,5]thiadiazepino[3,4,5-hi]indole-9-carboxamide 2,2-dioxide;
7-Ethyl-N-[(1S,2R)-2-hydroxy-3-({1-methyl-1-[3-(methyloxy)phenyl]ethyl}amino)-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-3-{[(1-ethyl-1H-pyrazol-4-yl)methyl]amino}-2-hydroxy-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-3-{[(1-ethyl-1H-pyrazol-4-yl)methyl]amino}-2-hydroxy-1-(phenylmethyl)propyl]-1,3-dimethyl-3,4-dihydro-1H-[1,2,5]thiadiazepino[3,4,5-hi]indole-9-carboxamide 2,2-dioxide;
7-Ethyl-N-[(1S,2R)-2-hydroxy-3-[(1-methylethyl)amino]-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-(tetrahydro-2H-pyran-4-ylamino)propyl]-1-methyl-3,4-dihydro-1H-[1,2,5]thiadiazepino[3,4,5-hi]indole-9-carboxamide 2,2-dioxide;
N-[(1S,2R)-3-(Cyclopropylamino)-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-(tetrahydro-2H-pyran-4-ylamino)propyl]-1-(1-methylethyl)-3,4-dihydro-1H-[1,2,5]thiadiazepino[3,4,5-hi]indole-9-carboxamide 2,2-dioxide;
1,7-Diethyl-N-[(1S,2R)-2-hydroxy-1-(phenylmethyl)-3-(tetrahydro-2H-pyran-4-ylamino)propyl]-3,4-dihydro-1H-[1,2,5]thiadiazepino[3,4,5-hi]indole-9-carboxamide 2,2-dioxide;
N-[(1S,2R)-2-Hydroxy-3-[(1-methylethyl)amino]-1-(phenylmethyl)propyl]-1-methyl-7-(1-methylethyl)-3,4-dihydro-1H-[1,2,5]thiadiazepino[3,4,5-hi]indole-9-carboxamide 2,2-dioxide;
N-[(1S,2R)-3-(Cyclohexylamino)-2-hydroxy-1-(phenylmethyl)propyl]-1-methyl-7-(1-methylethyl)-3,4-dihydro-1H-[1,2,5]thiadiazepino[3,4,5-hi]indole-9-carboxamide 2,2-dioxide;
N-[(1S,2R)-3-(Cyclopropylamino)-2-hydroxy-1-(phenylmethyl)propyl]-1-methyl-7-(1-methylethyl)-3,4-dihydro-1H-[1,2,5]thiadiazepino[3,4,5-hi]indole-9-carboxamide 2,2-dioxide;
N-[(1S,2R)-2-Hydroxy-1-(phenylmethyl)-3-(tetrahydro-2H-pyran-4-ylamino)propyl]-1-methyl-7-(1-methylethyl)-3,4-dihydro-1H-[1,2,5]thiadiazepino[3,4,5-hi]indole-9-carboxamide 2,2-dioxide;
N-[(1S,2R)-2-Hydroxy-3-({[3-(methyloxy)phenyl]methyl}amino)-1-(phenylmethyl)propyl]-1-methyl-7-(1-methylethyl)-3,4-dihydro-1H-[1,2,5]thiadiazepino[3,4,5-hi]indole-9-carboxamide 2,2-dioxide;
N-[(1S,2R)-3-(Cyclohexylamino)-2-hydroxy-1-(phenylmethyl)propyl]-1,7-diethyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(2,2,2-trifluoroethyl)amino]propyl}-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-[(2,2,3,3,3-pentafluoropropyl)amino]-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-3-[(Cyclopropylmethyl)amino]-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
N-[(1S,2R)-1-[(Chlorophenyl)methyl]-3-(cyclopropylamino)-2-hydroxypropyl]-7-ethyl-1-methyl-3,4-dihydro-1H-[1,2,5]thiadiazepino[3,4,5-hi]indole-9-carboxamide 2,2-dioxide;
N-[(1S,2R)-1-[(3-Chlorophenyl)methyl]-3-(cyclohexylamino)-2-hydroxypropyl]-7-ethyl-1-methyl-3,4-dihydro-1H-[1,2,5]thiadiazepino[3,4,5-hi]indole-9-carboxamide 2,2-dioxide;
N-[(1S,2R)-1-[(3-Chlorophenyl)methyl]-2-hydroxy-3-(tetrahydro-2H-pyran-4-ylamino)propyl]-7-ethyl-1-methyl-3,4-dihydro-1H-[1,2,5]thiadiazepino[3,4,5-hi]indole-9-carboxamide 2,2-dioxide;
N-{(1S,2R)-3-(Cyclopropylamino)-1-[(3-fluorophenyl)methyl]-2-hydroxypropyl}-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-(tetrahydro-2H-pyran-4-ylamino)propyl]-1-(2,2,2-trifluoroethyl)-3,4-dihydro-1H-[1,2,5]thiadiazepino[3,4,5-hi]indole-9-carboxamide 2,2-dioxide;
N-{(1S,2R)-3-(Cyclohexylamino)-1-[(3-fluorophenyl)methyl]-2-hydroxypropyl}-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-1-[(3-fluorophenyl)methyl]-2-hydroxy-3-(tetrahydro-2H-pyran-4-ylamino)propyl]-1-methyl-3,4-dihydro-1H-[1,2,5]thiadiazepino[3,4,5-hi]indole-9-carboxamide 2,2-dioxide;
N-{(1S,2R)-3-(Cyclohexylamino)-1-[(3,5-difluorophenyl)methyl]-2-hydroxypropyl}-7-ethyl-1-methyl-3,4-dihydro-1H-[1,2,5]thiadiazepino[3,4,5-hi]indole-9-carboxamide 2,2-dioxide;
N-{(1S,2R)-3-(Cyclopropylamino)-1-[(3,5-difluorophenyl)methyl]-2-hydroxypropyl}-7-ethyl-1-methyl-3,4-dihydro-1H-[1,2,5]thiadiazepino[3,4,5-hi]indole-9-carboxamide 2,2-dioxide;
N-[(1S,2R)-3-(Cyclobutylamino)-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-3-[(2-fluoroethyl)amino]-2-hydroxy-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-3-[(2,2-Dimethyltetrahydro-2H-pyran4-yl)amino]-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
N-[(1S,2R)-3-[(1,1-Dimethylethyl)amino]-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
N-[(1S,2R)-2-Hydroxy-1-(phenylmethyl)-3-({[3-(trifluoromethyl)phenyl]methyl}amino)propyl]-1-methyl-7-propyl-3,4-dihydro-1H-[1,2,5]thiadiazepino[3,4,5-hi]indole-9-carboxamide 2,2-dioxide;
N-[(1S,2R)-3-(Cyclohexylamino)-2-hydroxy-1-(phenylmethyl)propyl]-1-methyl-7-propyl-3,4-dihydro-1H-[1,2,5]thiadiazepino[3,4,5-hi]indole-9-carboxamide 2,2-dioxide;
N-[(1S,2R)-2-Hydroxy-1-(phenylmethyl)-3-(tetrahydro-2H-pyran-4-ylamino)propyl]-1-methyl-7-propyl-3,4-dihydro-1H-[1,2,5]thiadiazepino[3,4,5-hi]indole-9-carboxamide 2,2-dioxide;
N-[(1S,2R)-3-{[(1-Ethyl-1H-pyrazol4-yl)methyl]amino}-2-hydroxy-1-(phenylmethyl)propyl]-1-methyl-7-propyl-3,4-dihydro-1H-[1,2,5]thiadiazepino[3,4,5-hi]indole-9-carboxamide 2,2-dioxide;
1-Ethyl-N-[(1S,2R)-2-hydroxy-1-(phenylmethyl)-3-({[3-(trifluoromethyl)phenyl]methyl}amino)propyl]-7-propyl-3,4-dihydro-1H-[1,2,5]thiadiazepino[3,4,5-hi]indole-9-carboxamide 2,2-dioxide;
N-[(1S,2R)-3-(Cyclohexylamino)-2-hydroxy-1-(phenylmethyl)propyl]-1-ethyl-7-propyl-3,4-dihydro-1H-[1,2,5]thiadiazepino[3,4,5-hi]indole-9-carboxamide 2,2-dioxide;
1-Ethyl-N-[(1S,2R)-2-hydroxy-1-(phenylmethyl)-3-(tetrahydro-2H-pyran-4-ylamino)propyl]-7-propyl-3,4-dihydro-1H-[1,2,5]thiadiazepino[3,4,5-hi]indole-9-carboxamide 2,2-dioxide;
1-Ethyl-N-[(1S,2R)-3-{[(1-ethyl-1H-pyrazol-4-yl)methyl]amino}-2-hydroxy-1-(phenylmethyl)propyl]-7-propyl-3,4-dihydro-1H-[1,2,5]thiadiazepino[3,4,5-hi]indole-9-carboxamide 2,2-dioxide;
N-[(1S,2R)-1-[(3,5-Difluorophenyl)methyl]-2-hydroxy-3-(tetrahydro-2H-pyran-4-ylamino)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[2-(methyloxy)ethyl]amino}-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-3-(ethylamino)-2-hydroxy-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[(1S)-1-methylpropyl]amino}-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-3-(Butylamino)-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-(2-propyn-1-ylamino)propyl]-1-methyl-3,4-dihydro-1H-[1,2,5]thiadiazepino[3,4,5-hi]indole-9-carboxamide 2,2-dioxide;
N-[(1S,2R)-3-(Cyclopentylamino)-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-[(2-methylpropyl)amino]-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-1-(phenylmethyl)-3-(propylamino)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[(1R)-1-methylpropyl]amino}-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-3-[(2,2-Difluoroethyl)amino]-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(phenylmethyl)amino]propyl}-1-methyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(2-pyridinylmethyl)amino]propyl}-1-methyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(4-pyridinylmethyl)amino]propyl}-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-[(2-phenylethyl)amino]-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[({3-[(trifluoromethyl)oxy]phenyl}methyl)amino]propyl}-1-methyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(3-pyridinylmethyl)amino]propyl}-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[(2-methylphenyl)methyl]amino}-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[(3-methylphenyl)methyl]amino}-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[(4-methylphenyl)methyl]amino}-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-3-[(1S)-2,3-Dihydro-1H-inden-1-ylamino]-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
1,1-Dimethylethyl [7-ethyl-9-({[(1S,2R)-2-hydroxy-1-(phenylmethyl)-3-({[3-(trifluoromethyl)phenyl]methyl}amino)propyl]amino}carbonyl)-2,2-dioxido-3,4-dihydro-1H-[1,2,5]thiadiazepino[3,4,5-hi]indol-1-yl]acetate;
7-Ethyl-N-[(1S,2R)-2-hydroxy-1-(phenylmethyl)-3-({[1-(2,2,2-trifluoroethyl)-1H-pyrazol-4-yl]methyl}amino)propyl]-1-methyl-3,4-dihydro-1H-[1,2,5]thiadiazepino[3,4,5-hi]indole-9-carboxamide 2,2-dioxide;
6-Ethyl-N-{(1S,2R)-2-hydroxy-1-(phenylmethyl)-3-[(1,1,5-trimethylhexyl)amino]propyl}-1H-[1,2,5]thiadiazino[3,4,5-hi]indole-8-carboxamide 2,2-dioxide;
N-[(1S,2R)-3-(Cyclohexylamino)-2-hydroxy-1-(phenylmethyl)propyl]-6-ethyl-1H-[1,2,5]thiadiazino[3,4,5-hi]indole-8-carboxamide 2,2-dioxide;
6-Ethyl-N-[(1S,2R)-2-hydroxy-1-(phenylmethyl)-3-({[3-(trifluoromethyl)phenyl]methyl}amino)propyl]-1H-[1,2,5]thiadiazino[3,4,5-hi]indole-8-carboxamide 2,2-dioxide;
6-Ethyl-N-[(1S,2R)-2-hydroxy-1-(phenylmethyl)-3-({[3-(trifluoromethyl)phenyl]methyl}amino)propyl]-1,3-dimethyl-1H-[1,2,5]thiadiazino[3,4,5-hi]indole-8-carboxamide 2,2-dioxide;
6-Ethyl-N-[(1S,2R)-2-hydroxy-3-({1-methyl-1-[3-(trifluoromethyl)phenyl]ethyl}amino)-1-(phenylmethyl)propyl]-1-methyl-1H-[1,2,5]thiadiazino[3,4,5-hi]indole-8-carboxamide 2,2-dioxide;
N-[(1S,2R)-3-(Cyclohexylamino)-2-hydroxy-1-(phenylmethyl)propyl]-6-ethyl-1-methyl-1H-[1,2,5]thiadiazino[3,4,5-hi]indole-8-carboxamide 2,2-dioxide;
6-Ethyl-N-[(1S,2R)-2-hydroxy-3-({1-methyl-1-[3-(methyloxy)phenyl]ethyl}amino)-1-(phenylmethyl)propyl]-1-methyl-1H-[1,2,5]thiadiazino[3,4,5-hi]indole-8-carboxamide 2,2-dioxide;
6-Ethyl-N-[(1S,2R)-2-hydroxy-1-(phenylmethyl)-3-(tetrahydro-2H-pyran-4-ylamino)propyl]-1-methyl-1H-[1,2,5]thiadiazino[3,4,5-hi]indole-8-carboxamide 2,2-dioxide;
6-Ethyl-N-[(1S,2R)-2-hydroxy-1-(phenylmethyl)-3-({[3-(trifluoromethyl)phenyl]methyl}amino)propyl]-1-methyl-1H-[1,2,5]thiadiazino[3,4,5-hi]indole-8-carboxamide 2,2-dioxide;
6-Ethyl-N-{(1S,2R)-2-hydroxy-1-(phenylmethyl)-3-[(1,1,5-trimethylhexyl)amino]propyl}-1-methyl-1H-[1,2,5]thiadiazino[3,4,5-hi]indole-8-carboxamide 2,2-dioxide;
6-Ethyl-N-[(1S,2R)-2-hydroxy-3-[(1-methylethyl)amino]-1-(phenylmethyl)propyl]-1-methyl-1H-[1,2,5]thiadiazino[3,4,5-hi]indole-8-carboxamide 2,2-dioxide;
6-Ethyl-N-[(1S,2R)-2-hydroxy-3-({[3-(methyloxy)phenyl]methyl}amino)-1-(phenylmethyl)propyl]-1-methyl-1H-[1,2,5]thiadiazino[3,4,5-hi]indole-8-carboxamide 2,2-dioxide;
6-Ethyl-N-[(1S,2R)-3-{[(1-ethyl-1H-pyrazol-4-yl)methyl]amino}-2-hydroxy-1-(phenylmethyl)propyl]-1-methyl-1H-[1,2,5]thiadiazino[3,4,5-hi]indole-8-carboxamide 2,2-dioxide;
6-Ethyl-N-[(1S,2R)-2-hydroxy-1-(phenylmethyl)-3-(tetrahydro-2H-pyran-4-ylamino)propyl]-1,3,3-trimethyl-1H-[1,2,5]thiadiazino[3,4,5-hi]indole-8-carboxamide 2,2-dioxide;
N-[(1S,2R)-3-Amino-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-(methylamino)-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-2-Hydroxy-3-(methylamino)-1-(phenylmethyl)propyl]-1-methyl-7-(1-methylethyl)-3,4-dihydro-1H-[1,2,5]thiadiazepino[3,4,5-hi]indole-9-carboxamide 2,2-dioxide;
N-[(1S,2R)-1-[(3-Chlorophenyl)methyl]-2-hydroxy-3-(methylamino)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-1-[(3-fluorophenyl)methyl]-2-hydroxy-3-(methylamino)propyl]-1-methyl-3,4-dihydro-1H-[1,2,5]thiadiazepino[3,4,5-hi]indole-9-carboxamide 2,2-dioxide;
6-Ethyl-N-[(1S,2R)-2-hydroxy-3-(methylamino)-1-(phenylmethyl)propyl]-1-methyl-1H-[1,2,5]thiadiazino[3,4,5-hi]indole-8-carboxamide 2,2-dioxide;
[7-Ethyl-9-({[(1S,2R)-2-hydroxy-1-(phenylmethyl)-3-({[3-(trifluoromethyl)phenyl]methyl}amino)propyl]amino}carbonyl)-2,2-dioxido-3,4-dihydro-1H-[1,2,5]thiadiazepino[3,4,5-hi]indol-1-yl]acetic acid;
N-[(1S,2R)-3-{[(6-Bromo-2-pyridinyl)methyl]amino}-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-[({5-[(methylamino)carbonyl]-3-pyridinyl}methyl)amino]-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-3-[(2,2′-Bipyridin-6-ylmethyl)amino]-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{([(6-methyl-2-quinoxalinyl)methyl]amino}-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-{(1S,2R)-2-hydroxy-1-(phenylmethyl)-3-[(3-quinolinylmethyl)amino]propyl}-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[(6-methyl-2-pyridinyl)methyl]amino}-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-3-{[(5-ethyl-3-thienyl)methyl]amino}-2-hydroxy-1-(phenylmethyl)propyl]-1-methyl-3,4-dihydro-1H-[1,2,5]thiadiazepino[3,4,5-hi]indole-9-carboxamide 2,2-dioxide;
7-Ethyl-N-[(1S,2R)-2-hydroxy-3-{[(5-methyl-2-pyrazinyl)methyl]amino}-1-(phenylmethyl)propyl]-1-methyl-3,4-dihydro-1H-[1,2,5]thiadiazepino[3,4,5-hi]indole-9-carboxamide 2,2-dioxide;
7Ethyl-N-[(1S,2R)-3-{[(3-ethyl-5-isoxazolyl)methyl]amino}-2-hydroxy-1-(phenylmethyl)propyl]-1-methyl-3,4-dihydro-1H-[1,2,5]thiadiazepino[3,4,5-hi]indole-9-carboxamide 2,2-dioxide;
N-[(1S,2R)-3-{[(1S)-2-(Cyclohexylamino)-1-methyl-2-oxoethyl]amino}-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide; or
N-[(1S,2R)-3-[(4,4-Difluorocyclohexyl)amino]-2-hydroxy-1-(phenylmethyl)propyl]-7-ethyl-1-methyl-3,4-dihydro-1H-[1,2,5]thiadiazepino[3,4,5-hi]indole-9-carboxamide 2,2-dioxide;
or a pharmaceutically acceptable salt or solvate thereof.

20. A pharmaceutical composition comprising a compound of formula (I) as defined in claim 9 or a pharmaceutically acceptable salt or solvate thereof in admixture with one or more pharmaceutically acceptable diluents or carriers.

21. A method of treatment or prophylaxis of diseases characterised by elevated β-amyloid levels or β-amyloid deposits which comprises administering to a patient an effective amount of a compound of formula (I) as defined in claim 9 or a pharmaceutically acceptable salt or solvate thereof.

22. A method according to claim 5, wherein the disease characterised by elevated β-amyloid levels or β-amyloid deposits is Alzheimer's disease.
